(19) United States
(12) Patent Application Publication
(10) Pub. No.: US 2020/0360364 A1
(43) Pub. Date: Nov. 19, 2020

(54) METHODS AND COMPOSITIONS FOR TREATING INFLAMMATORY OR AUTOIMMUNE DISEASES OR CONDITIONS USING CHRNA6 ACTIVATORS

(71) Applicant: Flagship Pioneering Innovations V, Inc., Cambridge, MA (US)

(72) Inventors: Avak KAHVEJIAN, Lexington, MA (US); Jordi MATA-FINK, Happy Valley, OR (US); Jonathan Barry HUROV, Bedford, MA (US); Chengyi Jenny SHU, Cambridge, MA (US); Manuel Andreas FANKHAUSER, Bern (CH); Julian Alexander STANLEY, Oregon City, OR (US)

(21) Appl. No.: 16/966,079

(22) PCT Filed: Jan. 31, 2019
(86) PCT No.: PCT/US19/16109

§ 371 Date: Jul. 30, 2020

Related U.S. Application Data
(60) Provisional application No. 62/624,269, filed on Jan. 31, 2018.

(51) Int. Cl. A61K 31/465

(52) U.S. Cl. CPC A61K 31/465; A61K 45/06

(57) ABSTRACT

The present invention provides methods for treating inflammatory or autoimmune diseases or conditions using activators tin of nicotinic acetylcholine receptors (nAChRs) containing a cholinergic receptor nicotinic alpha 6 subunit (nAChRαS), such as activating antibodies that bind to a nAChR containing a nAChPα6 subunit and small molecule agonists of nAChRs containing a nAChRαS subunit. The invention also features compositions containing α6*nAChR activators, methods of diagnosing patients with an α6*nAChR-associated inflammatory or autoimmune disease or condition, and methods of predicting the response of an inflammatory or autoimmune disease or condition in a subject to treatment with α6*nAChR activators.

### BACKGROUND

Epidemiological data provide evidence of a steady rise in inflammatory and autoimmune disease throughout westernized societies over the last decades. The net % increase/year incidence and prevalence of autoimmune diseases worldwide have been reported to be 19% and 12%, respectively (Lerner et al., Intl J Celiac Dis. 3:151, 2015). Thus, there remains a need in the field for treatments of immune conditions such as autoimmune disease.

### SUMMARY OF THE INVENTION

The present invention provides methods for treating inflammatory or autoimmune diseases or conditions using activators of nicotinic acetylcholine receptors (nAChRs) containing a cholinergic receptor nicotinic alpha 6 subunit (nAChRα6), such as α6*nAChR activating antibodies and small molecule α6*nAChR activators, among others. The subunit is referred to as “nAChRα6,” while receptors containing the subunit are collectively referred to herein as “α6*nAChRs.” The invention also features compositions containing α6*nAChR activators, methods of diagnosing patients with an α6*nAChR-associated inflammatory or autoimmune disease or condition, and methods of predicting the response of an inflammatory or autoimmune disease or condition in a subject to treatment with α6*nAChR activators.

In a first aspect, the invention provides a method of modulating an immune response in a subject in need thereof by administering an effective amount of an α6*nAChR activator.

In another aspect, the invention provides a method of modulating an immune response in a subject in need thereof by contacting an immune cell, spleen, lymph node, secondary lymphoid organ, tertiary lymphoid organ, barrier tissue, skin, gut, or airway with an effective amount of an α6*nAChR activator.

In another aspect, the invention provides a method of modulating an immune cell activity in a subject in need thereof by contacting an immune cell, spleen, lymph node, secondary lymphoid organ, tertiary lymphoid organ, barrier tissue, skin, gut, or airway with an effective amount of an α6*nAChR activator.

In another aspect, the invention provides a method of modulating an immune cell activity in a subject in need thereof by contacting an immune cell, spleen, lymph node, secondary lymphoid organ, tertiary lymphoid organ, barrier tissue, skin, gut, or airway with an effective amount of an α6*nAChR activator.

In another aspect, the invention provides a method of treating a subject with an inflammatory or autoimmune disease or condition, by administering to the subject an effective amount of an α6*nAChR activator.

In another aspect, the invention provides a method of treating a subject with an inflammatory or autoimmune disease or condition by contacting an immune cell, spleen, lymph node, secondary lymphoid organ, tertiary lymphoid organ, barrier tissue, skin, gut, or airway with an effective amount of an α6*nAChR activator.

In another aspect, the invention provides a method of treating a subject identified as having an inflammatory or autoimmune disease or condition by administering to the subject an effective amount of an α6*nAChR activator.

In another aspect, the invention provides a method of treating a subject identified as having an inflammatory or autoimmune disease or condition by contacting an immune cell, spleen, lymph node, secondary lymphoid organ, tertiary lymphoid organ, barrier tissue, skin, gut, or airway with an effective amount of an α6*nAChR activator.

In another aspect, the invention provides a method of decreasing levels of one or more pro-inflammatory cytokine in a subject in need thereof by administering to the subject an effective amount of an α6*nAChR activator. In some embodiments, the subject is a subject with an α6*nAChR-associated inflammatory or autoimmune disease or condition. In some embodiments, the one or more pro-inflammatory cytokine includes interferon gamma (IFNγ). In some embodiments, the method further includes determining the level of one or more pro-inflammatory cytokine after administration of the α6*nAChR activator.

In another aspect, the invention provides a method of increasing levels of one or more anti-inflammatory cytokine in a subject in need thereof by administering to the subject an effective amount of an α6*nAChR activator. In some embodiments, the subject is a subject with an α6*nAChR-associated inflammatory or autoimmune disease or condition. In some embodiments, the one or more anti-inflammatory cytokine includes interleukin-10 and/or transforming growth factor beta (TGFβ). In some embodiments, the method further includes determining the level of one or more anti-inflammatory cytokine after administration of the α6*nAChR activator.

In another aspect, the invention provides a method of decreasing T cell activation in a subject in need thereof by administering to the subject an effective amount of an α6*nAChR activator. In some embodiments, the subject is a subject with α6*nAChR-associated cancer. In some embodiments, the method further includes evaluating T cell activation after administration of the α6*nAChR activator.

In some embodiments of any of the above aspects, the inflammatory or autoimmune disease or condition is an α6*nAChR-associated inflammatory or autoimmune disease or condition.

In another aspect, the invention provides a method of treating a subject with an inflammatory or autoimmune disease or condition by: a) identifying a subject with α6*nAChR-associated inflammatory or autoimmune disease or condition; and b) administering to the subject an effective amount of an α6*nAChR activator.

In another aspect, the invention provides a method of treating a subject with an inflammatory or autoimmune disease or condition by: a) identifying a subject with α6*nAChR-associated inflammatory or autoimmune disease or condition; and b) contacting an immune cell, spleen, lymph node, secondary lymphoid organ, tertiary lymphoid organ, barrier tissue, skin, gut, or airway with an effective amount of an α6*nAChR activator.

In another aspect, the invention provides a method of treating a subject with an α6*nAChR-associated inflammatory or autoimmune disease or condition by administering to the subject an effective amount of an α6*nAChR activator.

In another aspect, the invention provides a method of treating a subject with an α6*nAChR-associated inflammatory or autoimmune disease or condition by contacting an immune cell, spleen, lymph node, secondary lymphoid organ, tertiary lymphoid organ, barrier tissue, skin, gut, or airway with an effective amount of an α6*nAChR activator.

In some aspects of any of the above embodiments, the method includes contacting an immune cell with an effective amount of an α6*nAChR activator. In some aspects of any of the above embodiments, the method includes contacting the spleen with an effective amount of an α6*nAChR activator. In some aspects of any of the above embodiments, the method includes contacting a lymph node with an effective amount of an α6*nAChR activator. In some aspects of any of the above embodiments, the method includes contacting a secondary lymphoid organ with an effective amount of an α6*nAChR activator. In some aspects of any of the above embodiments, the method includes contacting a tertiary lymphoid organ with an effective amount of an α6*nAChR activator. In some aspects of any of the above embodiments, the method includes contacting a barrier tissue with an effective amount of an α6*nAChR activator. In some aspects of any of the above embodiments, the method includes contacting the skin with an effective amount of an α6*nAChR activator. In some aspects of any of the above embodiments, the method includes contacting the gut with an effective amount of an α6*nAChR activator. In some aspects of any of the above embodiments, the method includes contacting an airway with an effective amount of an α6*nAChR activator.

In another aspect, the invention provides a method of increasing regulatory T cell (Treg) production of one or more anti-inflammatory cytokine by contacting a Treg with an effective amount of an α6*nAChR activator. In some embodiments, the Treg is a Treg expressing α6*nAChR (e.g., the CHRNA6 gene or nAChRα6 subunit protein). In some embodiments, the one or more anti-inflammatory cytokine includes IL-10 and/or TGFβ.

In another aspect, the invention provides a method of decreasing T cell production of one or more pro-inflammatory cytokine by contacting a Treg with an effective amount of an α6*nAChR activator. In some embodiments, the Treg is a Treg expressing α6*nAChR (e.g., the CHRNA6 gene or nAChRα6 subunit protein). In some embodiments, the one or more pro-inflammatory cytokine includes IFNγ.

In another aspect, the invention provides a method of increasing T cell activation by contacting a Treg with an effective amount of an α6*nAChR activator. In some embodiments, the Treg is a Treg expressing α6*nAChR (e.g., the CHRNA6 gene or nAChRα6 subunit protein).

In some embodiments of any of the above aspects, the α6*nAChR-associated inflammatory or autoimmune disease or condition is associated with expression (e.g., gene or protein expression) of α6*nAChR in immune cells (e.g., regulatory T cells (Tregs)). In some embodiments of any of the above aspects, the α6*nAChR-associated inflammatory or autoimmune disease or condition is associated with decreased expression (e.g., gene or protein underexpression) of α6*nAChR in immune cells (e.g., Tregs).

In some embodiments of any of the above aspects, the method includes contacting an immune cell with an effective amount of an α6*nAChR activator that increases expression or activity of α6*nAChR in the immune cell.

In some embodiments of any of the above aspects, the method includes modulating an immune cell activity.

In some embodiments of any of the above aspects, the immune cell activity is activation, proliferation, polarization, cytokine production, recruitment, migration, phagocytosis, antibody-dependent cell-mediated cytotoxicity (ADCC), antibody-dependent cell-mediated phagocytosis (ADCP), antigen presentation, lymph node homing, lymph node egress, differentiation, or degranulation. In some embodiments, the immune cell activity is cytokine production. In some embodiments, the immune cell activity is activation.

In some embodiments, immune cell (e.g., Treg) activation, proliferation, polarization, cytokine production, recruitment, or migration is increased. In some embodiments, migration is directed toward a site of inflammation. In some embodiments, recruitment or migration is directed toward a lymph node or secondary lymphoid organ.

In some embodiments of any of the above aspects, the cytokine is an anti-inflammatory cytokine. In some embodiments of any of the above aspects, the anti-inflammatory cytokine is IL-10 and/or transforming growth factor beta (TGF-β).

In some embodiments, immune cell (e.g., T cell) activation, proliferation, polarization, cytokine production, recruitment, migration, ADCC, or antigen presentation is decreased. In some embodiments the cytokine is a pro-inflammatory cytokine. In some embodiments, the pro-inflammatory cytokine is IFNγ

In another aspect, the invention provides a method of increasing regulatory T cell (Treg) cytokine production in a subject in need thereof by contacting a Treg with an effective amount of an α6*nAChR activator.

In another aspect, the invention provides a method of increasing Treg cytokine production in subject in need thereof by administering to the subject an effective amount of an α6*nAChR activator.

In another aspect, the invention provides a method of increasing Treg cytokine production in a cell by contacting a Treg with an effective amount of an α6*nAChR activator.

In another aspect, the invention provides a method of increasing Treg cytokine production in a cell by administering an effective amount of an α6*nAChR activator.

In some embodiments of any of the above aspects, the method increases Treg production of anti-inflammatory cytokines.

In some embodiments of any of the above aspects, the anti-inflammatory cytokines are IL-10 and/or TGFβ.

In another aspect, the invention provides a method of decreasing T cell cytokine production in a subject in need thereof by administering to the subject an effective amount of an α6*nAChR activator. In some embodiments, the cytokine is a pro-inflammatory cytokine. In some embodiments, the cytokine is IFNγ.

In another aspect, the invention provides a method of decreasing T cell activation in a subject in need thereof by administering to the subject an effective amount of an α6*nAChR activator.

In some embodiments of any of the above aspects, the method further includes contacting an immune cell isolated from the subject with an α6*nAChR activator and evaluating the response of the immune cell prior to administration of the α6*nAChR activator.

In another aspect, the invention provides a method of treating a subject with an inflammatory or autoimmune disease or condition, the method including the steps of a) contacting an immune cell isolated from the subject with an α6*nAChR activator and evaluating a response of the immune cell; and b) administering to the subject an effective amount of an α6*nAChR activator if the response of the immune cell is modulated by the α6*nAChR activator.

In another aspect, the invention provides a method of treating a subject with an inflammatory or autoimmune disease or condition, the method including the steps of a) contacting an immune cell isolated from the subject with an α6*nAChR activator and evaluating a response of the immune cell; and b) contacting an immune cell, a tumor, a tumor microenvironment, a site of metastasis, a lymph node, a spleen, a secondary lymphoid organ, or a tertiary lymphoid organ with an effective amount of an α6*nAChR activator if the response of the immune cell is modulated by the α6*nAChR activator.

In another aspect, the invention provides a method of treating a subject with an inflammatory or autoimmune disease or condition, the method including the steps of a) contacting an immune cell isolated from the subject with an α6*nAChR activator and evaluating a response of the immune cell; and b) administering to the subject an effective amount of an α6*nAChR activator.

In some embodiments of any of the above aspects, the immune cell is a Treg. In some embodiments of any of the above aspects, the response is Treg anti-inflammatory cytokine production. In some embodiments, the anti-inflammatory cytokine is IL-10 or TGFβ. In some embodiments of any of the above aspects, the response is Treg activation. In some embodiments of any of the above aspects, the response is Treg proliferation. In some embodiments of any of the above aspects, the response is Treg α6*nAChR expression or activity.

In another aspect, the invention provides a method of treating a subject with an inflammatory or autoimmune disease or condition, the method including the steps of a) contacting Treg isolated from the subject with an α6*nAChR activator; b) evaluating a response of a T cell (e.g., a CD8 T cell) that is co-cultured with the Treg; and c) administering to the subject an effective amount of an α6*nAChR activator if the response of the T cell is modulated by the α6*nAChR activator.

In another aspect, the invention provides a method of treating a subject with an inflammatory or autoimmune disease or condition, the method including the steps of a) contacting Treg isolated from the subject with an α6*nAChR activator; b) evaluating a response of a T cell (e.g., a CD8 T cell) that is co-cultured with the Treg; and c) contacting an immune cell, a tumor, a tumor microenvironment, a site of metastasis, a lymph node, a spleen, a secondary lymphoid organ, or a tertiary lymphoid organ with an effective amount of an α6*nAChR activator if the response of the T cell is modulated by the α6*nAChR activator.

In another aspect, the invention provides a method of treating a subject with an inflammatory or autoimmune disease or condition, the method including the steps of a) contacting Treg isolated from the subject with an α6*nAChR activator; b) evaluating a response of a T cell (e.g., a CD8 T cell) that is co-cultured with the Treg; and c) administering to the subject an effective amount of an α6*nAChR activator.

In some embodiments of any of the above aspects, the response is T cell pro-inflammatory cytokine production. In some embodiments, the pro-inflammatory cytokine is IFNγ. In some embodiments of any of the above aspects, the response is T cell activation. In some embodiments of any of the above aspects, the response is T cell proliferation.

In another aspect, the invention provides a method of predicting the response of an inflammatory or autoimmune disease or condition in a subject to treatment with an α6*nAChR activator by contacting an immune cell isolated from the subject with an α6*nAChR activator and evaluating the response of the immune cell.

In some embodiments of any of the above aspects, the evaluating includes assessing immune cell migration, immune cell proliferation, immune cell recruitment, immune cell differentiation, immune cell activation, immune cell polarization, immune cell cytokine production, immune cell degranulation, immune cell maturation, immune cell ADCC, immune cell ADCP, immune cell antigen presentation, or immune cell nAChRα6 expression. In some embodiments of any of the above aspects, the immune cell is a Treg. In some embodiments of any of the above aspects, the evaluating includes assessing Treg anti-inflammatory cytokine production. In some embodiments, the anti-inflammatory cytokine is IL-10 or TGFβ. In some embodiments of any of the above aspects, the evaluating includes assessing Treg activation. In some embodiments of any of the above aspects, the evaluating includes assessing Treg proliferation. In some embodiments, the response is Treg nAChRα6 expression or activity.

In another aspect, the invention provides a method of predicting the response of an inflammatory or autoimmune disease or condition in a subject to treatment with an α6*nAChR activator by: a) isolating an immune cell from the subject; b) measuring the expression of nAChRα6 in the immune cell; and c) comparing nAChRα6 expression in the immune cell to a reference, wherein decreased expression of nAChRα6 in the immune cell as compared to the reference indicates that the subject will respond to treatment with an α6*nAChR activator.

In some embodiments of any of the above aspects, the method further includes contacting the immune cell with an α6*nAChR activator.

In another aspect, the invention provides a method of characterizing an inflammatory or autoimmune disease or condition in a subject by: a) isolating an immune cell from the subject; b) measuring the expression of nAChRα6 in the immune cell; and c) comparing nAChRα6 expression in the immune cell to a reference, wherein decreased expression of nAChRα6 in the immune cell as compared to the reference indicates that the subject has an α6*nAChR-associated inflammatory or autoimmune disease or condition.

In another aspect, the invention provides a method of identifying a subject as having an α6*nAChR-associated inflammatory or autoimmune disease or condition by: a) isolating an immune cell from the subject; b) measuring the expression of nAChRα6 in the immune cell; and c) comparing nAChRα6 expression in the immune cell to a reference, wherein decreased expression of nAChRα6 in the immune cell as compared to the reference indicates that the subject has an α6*nAChR-associated inflammatory or autoimmune disease or condition.

In some embodiments, of any of the above aspects, the immune cell is a Treg.

In some embodiments of any of the above aspects, the method further includes providing an α6*nAChR activator suitable for administration to the subject. In some embodiments of any of the above aspects, the method further includes administering to the subject an effective amount of an α6*nAChR activator.

In some embodiments of any of the above aspects, the α6*nAChR activator induces or increases α6*nAChR channel opening or activity.

In some embodiments of any of the above aspects, the inflammatory or autoimmune disease or condition is systemic lupus erythematosus (SLE), rheumatoid arthritis, multiple sclerosis (MS), irritable bowel disorder (IBD), Crohn's disease, ulcerative colitis, dermatitis, psoriasis, or asthma. In some embodiments of any of the above aspects, the inflammatory or autoimmune disease or condition is SLE. In some embodiments of any of the above aspects, the inflammatory or autoimmune disease or condition rheumatoid arthritis. In some embodiments of any of the above aspects, the inflammatory or autoimmune disease or condition is MS. In some embodiments of any of the above aspects, the inflammatory or autoimmune disease or condition is IBD. In some embodiments of any of the above aspects, the inflammatory or autoimmune disease or condition is Crohn's disease. In some embodiments of any of the above aspects, the inflammatory or autoimmune disease or condition is ulcerative colitis. In some embodiments of any of the above aspects, the inflammatory or autoimmune disease or condition is dermatitis. In some embodiments of any of the above aspects, the inflammatory or autoimmune disease or condition is psoriasis. In some embodiments of any of the above aspects, the inflammatory or autoimmune disease or condition is asthma.

In some embodiments of any of the above aspects, the inflammatory or autoimmune disease or condition is an IFNγ-associated inflammatory or autoimmune disease or condition. In some embodiments of any of the above aspects, the IFNγ-associated inflammatory or autoimmune disease or condition is agammaglobulinemia. In some embodiments of any of the above aspects, the IFNγ-associated inflammatory or autoimmune disease or condition is autoimmune aplastic anemia. In some embodiments of any of the above aspects, the IFNγ-associated inflammatory or autoimmune disease or condition is autoimmune gastric atrophy. In some embodiments of any of the above aspects, the IFNγ-associated inflammatory or autoimmune disease or condition is cardiomyopathy. In some embodiments of any of the above aspects, the IFNγ-associated inflammatory or autoimmune disease or condition is hemolytic anemia. In some embodiments of any of the above aspects, the IFNγ-associated inflammatory or autoimmune disease or condition is lichen planus. In some embodiments of any of the above aspects, the IFNγ-associated inflammatory or autoimmune disease or condition is leukocytoclastic vasculitis. In some embodiments of any of the above aspects, the IFNγ-associated inflammatory or autoimmune disease or condition is linear IgA disease (LAD). In some embodiments of any of the above aspects, the IFNγ-associated inflammatory or autoimmune disease or condition is SLE. In some embodiments of any of the above aspects, the IFNγ-associated inflammatory or autoimmune disease or condition is multiple sclerosis. In some embodiments of any of the above aspects, the IFNγ-associated inflammatory or autoimmune disease or condition is myasthenia gravis. In some embodiments of any of the above aspects, the IFNγ-associated inflammatory or autoimmune disease or condition is mixed connective tissue disease (MCTD). In some embodiments of any of the above aspects, the IFNγ-associated inflammatory or autoimmune disease or condition is myositis. In some embodiments of any of the above aspects, the IFNγ-associated inflammatory or autoimmune disease or condition is polymyositis. In some embodiments of any of the above aspects, the IFNγ-associated inflammatory or autoimmune disease or condition is psoriasis. In some embodiments of any of the above aspects, the IFNγ-associated inflammatory or autoimmune disease or condition is plaque psoriasis. In some embodiments of any of the above aspects, the IFNγ-associated inflammatory or autoimmune disease or condition is pure red cell aplasia. In some embodiments of any of the above aspects, the IFNγ-associated inflammatory or autoimmune disease or condition is vesiculobullous dermatosis. In some embodiments of any of the above aspects, the IFNγ-associated inflammatory or autoimmune disease or condition is vasculitis. In some embodiments of any of the above aspects, the IFNγ-associated inflammatory or autoimmune disease or condition is vitiligo.

In some embodiments of any of the above aspects, the inflammatory or autoimmune disease or condition is an inflammatory or autoimmune disease or condition associated with activated T cells. In some embodiments of any of the above aspects, the inflammatory or autoimmune disease or condition associated with activated T cells is alopecia areata. In some embodiments of any of the above aspects, the inflammatory or autoimmune disease or condition associated with activated T cells is autoimmune aplastic anemia. In some embodiments of any of the above aspects, the inflammatory or autoimmune disease or condition associated with activated T cells is autoimmune myocarditis. In some embodiments of any of the above aspects, the inflammatory or autoimmune disease or condition associated with activated T cells is autoimmune retinopathy. In some embodiments of any of the above aspects, the inflammatory or autoimmune disease or condition associated with activated T cells is autoimmune thrombocytopenic purpura (ATP). In some embodiments of any of the above aspects, the inflammatory or autoimmune disease or condition associated with activated T cells is celiac disease. In some embodiments of any of the above aspects, the inflammatory or autoimmune disease or condition associated with activated T cells is collagen-induced arthritis. In some embodiments of any of the above aspects, the inflammatory or autoimmune disease or condition associated with activated T cells is dermatomyositis. In some embodiments of any of the above aspects, the inflammatory or autoimmune disease or condition associated with activated T cells is Devic's disease. In some embodiments of any of the above aspects, the inflammatory or autoimmune disease or condition associated with activated T cells is eosinophilic esophagitis. In some embodiments of any of the above aspects, the inflammatory or autoimmune disease or condition associated with activated T cells is giant cell myocarditis. In some embodiments of any of the above aspects, the inflammatory or autoimmune disease or condition associated with activated T cells is Evans syndrome. In some embodiments of any of the above aspects, the inflammatory or autoimmune disease or condition associated with activated T cells is glomerulonephritis. In some embodiments of any of the above aspects, the inflammatory or autoimmune disease or condition associated with activated T cells is autoimmune inner ear disease.

In some embodiments of any of the above aspects, the inflammatory or autoimmune disease or condition is an α6*nAChR-associated inflammatory or autoimmune disease or condition.

In some embodiments of any of the above aspects, the α6*nAChR activator is administered locally. In some embodiments of any of the above aspects, the α6*nAChR activator is administered to or near a lymph node, the spleen, a secondary lymphoid organ, a tertiary lymphoid organ, barrier tissue, skin, the gut, or an airway. In some embodiments of any of the above aspects, the α6*nAChR activator is administered to or near a lymph node. In some embodiments of any of the above aspects, the α6*nAChR activator is administered to or near the spleen. In some embodiments of any of the above aspects, the α6*nAChR activator is administered to or near a secondary lymphoid organ. In some embodiments of any of the above aspects, the α6*nAChR activator is administered to or near a tertiary lymphoid organ. In some embodiments of any of the above aspects, the α6*nAChR activator is administered to or near a barrier tissue. In some embodiments of any of the above aspects, the α6*nAChR activator is administered to or near the skin. In some embodiments of any of the above aspects, the α6*nAChR activator is administered to or near the gut. In some embodiments of any of the above aspects, the α6*nAChR activator is administered to or near an airway.

In some embodiments of any of the above aspects, the method further includes administering a second therapeutic agent.

In some embodiments of any of the above aspects, the α6*nAChR activator increases Treg migration, increases Treg proliferation, increases Treg recruitment, increases Treg activation, increases Treg polarization, or increases Treg cytokine production (e.g., Treg production of IL-10 and/or TGFβ), increases Treg α6*nAChR expression or activity, decreases T cell migration, decreases T cell proliferation, decreases T cell recruitment, decreases T cell activation, decreases T cell polarization, decreases T cell ADCC, decreases T cell antigen presentation, decreases T cell pro-inflammatory cytokine production, decreases inflammation, decreases auto-antibody levels, increases organ function, and/or decreases the rate or number of relapses or flare-ups. In some embodiments of any of the above aspects, the α6*nAChR activator increases Treg activation. In some embodiments of any of the above aspects, the α6*nAChR activator increases Treg anti-inflammatory cytokine production. In some embodiments of any of the above aspects, the anti-inflammatory cytokine is IL-10 or TGFβ. In some embodiments of any of the above aspects, the α6*nAChR activator increases Treg α6*nAChR expression. In some embodiments of any of the above aspects, the α6*nAChR activator decreases T cell activation. In some embodiments of any of the above aspects, the α6*nAChR activator decreases T cell pro-inflammatory cytokine production (e.g., increases production of IFNγ).

In some embodiments of any of the above aspects, the method further includes measuring one or more of the development of high endothelial venules (HEVs) or tertiary lymphoid organs (TLOs), immune cell migration, immune cell proliferation, immune cell recruitment, immune cell differentiation, immune cell activation, immune cell polarization, immune cell cytokine production, immune cell ADCC, immune cell ADCP, symptoms of an autoimmune or inflammatory condition, inflammation, auto-antibody levels, organ function, the rate or number of relapses or flare-ups, or immune cell nAChRα6 expression before administration of the α6*nAChR activator. In some embodiments of any of the above aspects, the method further includes measuring immune cell activation before administration of the α6*nAChR activator. In some embodiments of any of the above aspects, the method further includes measuring immune cell anti-inflammatory cytokine production before administration of the α6*nAChR activator.

In some embodiments of any of the above aspects, the method further includes measuring one or more of the development of HEVs or TLOs, immune cell migration, immune cell proliferation, immune cell recruitment, immune cell differentiation, immune cell activation, immune cell polarization, immune cell cytokine production, immune cell ADCC, immune cell ADCP, symptoms of an autoimmune or inflammatory condition, inflammation, auto-antibody levels, organ function, the rate or number of relapses or flare-ups, or nAChRα6 expression after administration of the α6*nAChR activator. In some embodiments of any of the above aspects, the method further includes measuring immune cell activation after administration of the α6*nAChR activator. In some embodiments of any of the above aspects, the method further includes measuring immune cell anti-inflammatory cytokine production after administration of the α6*nAChR activator.

In some embodiments of any of the above aspects, immune cell activation, immune cell proliferation, and/or immune cell polarization are measured based on expression of one or more immune cell markers.

In some embodiments of any of the above aspects, the one or more immune cell markers is a marker listed in Table 2. In some embodiments of any of the above aspects, the α6*nAChR activator is administered in an amount sufficient to increase Treg migration, increase Treg proliferation, increase Treg recruitment, increase Treg activation, increase Treg polarization, increase Treg anti-inflammatory cytokine production (e.g., increase Treg production of IL-10 and TGFβ), increase Treg nAChRα6 expression or activity, decrease T cell migration, decrease T cell proliferation, decrease T cell recruitment, decrease T cell activation, decrease T cell polarization, decrease T cell ADCC, decrease T cell antigen presentation, decrease T cell pro-inflammatory cytokine production, treat the autoimmune or inflammatory condition, reduce symptoms of an autoimmune or inflammatory condition, reduce inflammation, reduce auto-antibody levels, increase organ function, and/or decrease the rate or number of relapses or flare-ups. In some embodiments of any of the above aspects, the α6*nAChR activator is administered in an amount sufficient to increase Treg activation. In some embodiments of any of the above aspects, the α6*nAChR activator is administered in an amount sufficient to increase Treg anti-inflammatory cytokine production. In some embodiments of any of the above aspects, the anti-inflammatory cytokine is IL-10 or TGFβ. In some embodiments of any of the above aspects, the α6*nAChR activator is administered in an amount sufficient to increase Treg nAChRα6 expression. In some embodiments of any of the above aspects, the α6*nAChR activator is administered in an amount sufficient to decrease T cell activation. In some embodiments of any of the above aspects, the α6*nAChR activator is administered in an amount sufficient to decrease T cell pro-inflammatory cytokine production (e.g., increases production of IFNγ).

In some embodiments of any of the above aspects, the method further includes monitoring the progression of the inflammatory or autoimmune disease or condition after administration of the α6*nAChR activator (e.g., monitoring one or more of organ function, inflammation, auto-antibody levels, the rate or number of relapses or flare-ups, development of HEVs or TLOs, immune cell migration, immune cell proliferation, immune cell recruitment, lymph node homing, lymph node egress, immune cell differentiation, immune cell activation, immune cell polarization, immune cell cytokine production, immune cell degranulation, immune cell maturation, ADCC, ADCP, and/or immune cell nAChRα6 expression).

In some embodiments of any of the above aspects, the immune cell is a Treg.

In some embodiments of any of the above aspects, the subject is a human.

In another aspect, the invention provides a therapy for treating an inflammatory or autoimmune disease or condition containing an α6*nAChR activator and a second agent selected from the group consisting of: a disease-modifying anti-rheumatic drug (DMARD), a biologic response modifier (a type of DMARD), a corticosteroid, a nonsteroidal anti-inflammatory medication (NSAID), prednisone, prednisolone, methylprednisolone, methotrexate, hydroxycholorquine, sulfasalazine, leflunomide, cyclophosphamide, azathioprine, tofacitinib, adalimumab, abatacept, anakinra, kineret, certolizumab, etanercept, golimumab, infliximab, rituximab tocilizumab, an antiviral compound, a nucleoside-analog reverse transcriptase inhibitor (NRTI), a non-nucleoside reverse transcriptase inhibitor (NNRTI), an antibacterial compound, an antifungal compound, an antiparasitic compound, 6-mercaptopurine, 6-thioguanine, alemtuzumab, aminosalicylates, antibiotics, anti-histamines, anti-TNFα, azathioprine, belimumab, beta interferon, calcineurin inhibitors, certolizumab, corticosteroids, cromolyn, cyclosporin A, cyclosporine, dimethyl fumarate, fingolimod, fumaric acid esters, glatiramer acetate, hydroxyurea, IFNγ, IL-11, leflunomide, leukotriene receptor antagonist, long-acting beta2 agonist, mitoxantrone, mycophenolate mofetil, natalizumab, ocrelizumab, pimecrolimus, probiotics, retinoids, salicylic acid, short-acting beta2 agonist, sulfasalazine, tacrolimus, teriflunomide, theophylline, ustekinumab, vedolizumab, a neurotransmission modulator, and a neuronal growth factor modulator.

In some aspects of any of the above embodiments, the α6*nAChR activator is an activating antibody (e.g., an agonist antibody) or an antigen binding fragment thereof.

In some embodiments of any of the above aspects, the α6*nAChR activator is a small molecule α6*nAChR activator (e.g., agonist). In some embodiments of any of the above aspects, the small molecule α6*nAChR activator (e.g., agonist) is a small molecule activator listed in Table 1.

In another aspect, the invention provides a pharmaceutical composition containing an α6*nAChR activating antibody (e.g., agonist antibody) or an antigen binding fragment thereof. In some embodiments of any of the above aspects, the α6*nAChR activating antibody agonizes α6*nAChR (e.g., induces or increases channel opening, stabilizes the channel in an open conformation, or increases α6*nAChR activation).

In some embodiments of the above aspects, the composition further includes a second therapeutic agent.

In some embodiments of any of the above aspects, the composition further includes a pharmaceutically acceptable excipient.

In some embodiments of any of the above aspects, the second therapeutic agent is a DMARD, a biologic response modifier (a type of DMARD), a corticosteroid, an NSAID, prednisone, prednisolone, methylprednisolone, methotrexate, hydroxycholorquine, sulfasalazine, leflunomide, cyclophosphamide, azathioprine, tofacitinib, adalimumab, abatacept, anakinra, kineret, certolizumab, etanercept, golimumab, infliximab, rituximab tocilizumab, an antiviral compound, a NRTI, a NNRTI, an antibacterial compound, an antifungal compound, an antiparasitic compound, 6-mercaptopurine, 6-thioguanine, alemtuzumab, aminosalicylates, antibiotics, anti-histamines, anti-TNFα, azathioprine, belimumab, beta interferon, calcineurin inhibitors, certolizumab, corticosteroids, cromolyn, cyclosporin A, cyclosporine, dimethyl fumarate, fingolimod, fumaric acid esters, glatiramer acetate, hydroxyurea, IFNγ, IL-11, leflunomide, leukotriene receptor antagonist, long-acting beta2 agonist, mitoxantrone, mycophenolate mofetil, natalizumab, ocrelizumab, pimecrolimus, probiotics, retinoids, salicylic acid, short-acting beta2 agonist, sulfasalazine, tacrolimus, teriflunomide, theophylline, ustekinumab, vedolizumab, a second α6*nAChR activator, a neurotransmission modulator, or a neuronal growth factor modulator.

In some embodiments of any of the above aspects, the neurotransmission modulator is neurotoxin listed in Table 9, or a modulator (e.g., agonist or antagonist) of a neurotransmitter receptor listed in Table 5 or a neurotransmitter listed in Table 6. In some embodiments, the modulator of a neurotransmitter receptor listed in Table 5 or a neurotransmitter listed in Table 6 is an agonist or antagonist listed in Tables 7A-7J or a modulator listed in Table 8.

In some embodiments of any of the above aspects, the neuronal growth factor modulator is an agonist or antagonist of a neuronal growth factor listed in Table 10 Table 10. In some embodiments, the modulator of a neuronal growth factor listed in Table 10 is an antibody listed in Table 11 or an agonist or antagonist listed in Table 12. In some embodiments, the modulator of a neuronal growth factor listed in Table 10 is selected from the group consisting of etanercept, thalidomide, lenalidomide, pomalidomide, pentoxifylline, bupropion, DOI, disitertide, and trabedersen.

In some embodiments of any of the above aspects, the α6*nAChR activator is selected from the group consisting of an antibody and a small molecule. In some embodiments, the antibody is an α6*nAChR activating antibody. In some embodiments, the small molecule is a small molecule α6*nAChR activator (e.g., agonist). In some embodiments, the small molecule α6*nAChR activator (e.g., agonist) is a small molecule activator listed in Table 1).

In some embodiments of any of the above aspects, the α6*nAChR activator does not cross the blood brain barrier. In some embodiments, the α6*nAChR activator has been modified to prevent blood brain barrier crossing by conjugation to a targeting moiety, formulation in a particulate delivery system, addition of a molecular adduct, or through modulation of its size, polarity, flexibility, or lipophilicity.

In some embodiments of any of the above aspects, the α6*nAChR activator does not have a direct effect on the central nervous system or gut.

In some embodiments of any of the above aspects, the immune cell is a Treg.

In some embodiments of any of the above aspects, the α6*nAChR activator increases Treg migration, increases Treg proliferation, increases Treg recruitment, increases Treg activation, increases Treg polarization, increases Treg cytokine production (e.g., increases Treg production of IL-10 and TGFβ), increases Treg expression of α6*nAChR, reduces symptoms of an autoimmune or inflammatory condition, reduces inflammation, reduces auto-antibody levels, increases organ function, or decreases rate or number of relapses or flare-ups. In some embodiments of any of the above aspects, the α6*nAChR activator increases Treg proliferation. In some embodiments of any of the above aspects, the α6*nAChR activator increases Treg proliferation. In some embodiments of any of the above aspects, the α6*nAChR activator increases Treg activation. In some embodiments of any of the above aspects, the α6*nAChR activator increases Treg anti-inflammatory cytokine production (e.g., Treg production of IL-10 and/or TGFβ). In some embodiments of any of the above aspects, the α6*nAChR activator increases Treg nAChRα6 expression.

In some embodiments of any of the above aspects, the α6*nAChR activator decreases pro-inflammatory immune cell migration, decreases pro-inflammatory immune cell proliferation, decreases pro-inflammatory immune cell recruitment, decreases pro-inflammatory immune cell activation, decreases pro-inflammatory immune cell polarization, decreases pro-inflammatory immune cell cytokine production (e.g., decreases production of pro-inflammatory cytokines), decreases ADCC, or decreases ADCP. In some embodiments, the pro-inflammatory immune cell is a CD8+ T cell, a CD4+ T cell, a natural killer cell, a macrophage, or a dendritic cell. In some embodiments of any of the above aspects, the pro-inflammatory immune cell is a CD8+ T cell. In some embodiments of any of the above aspects, the α6*nAChR activator decreases T cell (e.g., CD8+ T cell) activation. In some embodiments of any of the above aspects, the α6*nAChR activator decreases T cell (e.g., CD8+ T cell) pro-inflammatory cytokine production (e.g., IFNγ production). In some embodiments of any of the above aspects, the effect of the α6*nAChR activator on pro-inflammatory immune cells is mediated by the effect of the α6*nAChR activator on Tregs.

### Definitions

As used herein, “administration” refers to providing or giving a subject a therapeutic agent (e.g., an α6*nAChR activator), by any effective route. Exemplary routes of administration are described herein below.

As used herein, the term “α6*nAChR” refers to nicotinic acetylcholine receptors (nAChRs) that contain a nAChRα6 subunit (e.g., one or more nAChRα6 subunit). The * indicates that other subunits may be present in the pentameric nAChR. For example, nAChRα6 is known to be found in nAChRs that contain nAChRα4, nAChRβ2, and/or nAChRβ3 subunits.

As used herein, the term “agonist” refers to an agent (e.g., a small molecule or antibody) that increases receptor activity. An agonist may activate a receptor by directly binding to the receptor, by acting as a cofactor, by modulating receptor conformation (e.g., maintaining a receptor in an open or active state). An agonist may increase receptor activity by 10%, 20%, 30%, 40%, 50%, 60%, 70%, 80%, 90%, 95%, 98% or more. An agonist may induce maximal receptor activation or partial activation depending on the concentration of the agonist and its mechanism of action.

As used herein, the term “analog” refers to a protein of similar nucleotide or amino acid composition or sequence to any of the proteins or peptides of the invention, allowing for variations that do not have an adverse effect on the ability of the protein or peptide to carry out its normal function (e.g., bind to a receptor or promote synapse formation). Analogs may be the same length, shorter, or longer than their corresponding protein or polypeptide. Analogs may have about 60% (e.g., about 60%, about 62%, about 64%, about 66%, about 68%, about 70%, about 72%, about 74%, about 76%, about 78%, about 80%, about 82%, about 84%, about 86%, about 88%, about 90%, about 92%, about 94%, about 96%, about 98%, or about 99%) identity to the amino acid sequence of the naturally occurring protein or peptide. An analog can be a naturally occurring protein or polypeptide sequence that is modified by deletion, addition, mutation, or substitution of one or more amino acid residues.

As used herein, the term “antagonist” refers to an agent (e.g., a small molecule or antibody) that reduces or inhibits receptor activity. An antagonist may reduce receptor activity by directly binding to the receptor, by blocking the receptor binding site, by modulating receptor conformation (e.g., maintaining a receptor in a closed or inactive state). An antagonist may reduce receptor activity by 10%, 20%, 30%, 40%, 50%, 60%, 70%, 80%, 90%, 95%, 98% or more. An antagonist may also completely block or inhibit receptor activity. Antagonist activity may be concentration-dependent or -independent.

As used herein, the term “antibody” refers to a molecule that specifically binds to, or is immunologically reactive with, a particular antigen and includes at least the variable domain of a heavy chain, and normally includes at least the variable domains of a heavy chain and of a light chain of an immunoglobulin. Antibodies and antigen-binding fragments, variants, or derivatives thereof include, but are not limited to, polyclonal, monoclonal, multispecific, human, humanized, primatized, or chimeric antibodies, heteroconjugate antibodies (e.g., bi- tri- and quad-specific antibodies, diabodies, triabodies, and tetrabodies), single-domain antibodies (sdAb), epitope-binding fragments, e.g., Fab, Fab′ and F(ab′)₂, Fd, Fvs, single-chain Fvs (scFv), rlgG, single-chain antibodies, disulfide-linked Fvs (sdFv), fragments including either a V_{L} or V_{H} domain, fragments produced by an Fab expression library, and anti-idiotypic (anti-Id) antibodies. Antibody molecules of the invention can be of any type (e.g., IgG, IgE, IgM, IgD, IgA, and IgY), class (e.g., IgG1, IgG2, IgG3, IgG4, IgA1 and IgA2) or subclass of immunoglobulin molecule. Moreover, unless otherwise indicated, the term “monoclonal antibody” (mAb) is meant to include both intact molecules as well as antibody fragments (such as, for example, Fab and F(ab′)2 fragments) that are capable of specifically binding to a target protein. Fab and F(ab′)2 fragments lack the Fc fragment of an intact antibody.

The term “antigen-binding fragment,” as used herein, refers to one or more fragments of an immunoglobulin that retain the ability to specifically bind to a target antigen. The antigen-binding function of an immunoglobulin can be performed by fragments of a full-length antibody. The antibody fragments can be a Fab, F(ab′)2, scFv, SMIP, diabody, a triabody, an affibody, a nanobody, an aptamer, or a domain antibody. Examples of binding fragments encompassed by the term “antigen-binding fragment” of an antibody include, but are not limited to: (i) a Fab fragment, a monovalent fragment consisting of the V_{L}, V_{H}, C_{L}, and C_{H}1 domains; (ii) a F(ab′)₂ fragment, a bivalent fragment including two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb (Ward et al., Nature 341:544-546, 1989) including V_{H} and V_{L} domains; (vi) a dAb fragment that consists of a V_{H} domain; (vii) a dAb that consists of a V_{H} or a V_{L} domain; (viii) an isolated complementarity determining region (CDR); and (ix) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a linker that enables them to be made as a single protein chain in which the V_{L} and V_{H} regions pair to form monovalent molecules (known as single chain Fv (scFv)). These antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in certain cases, by chemical peptide synthesis procedures known in the art.

As used herein, the term “cell type” refers to a group of cells sharing a phenotype that is statistically separable based on gene expression data. For instance, cells of a common cell type may share similar structural and/or functional characteristics, such as similar gene activation patterns and antigen presentation profiles. Cells of a common cell type may include those that are isolated from a common tissue (e.g., epithelial tissue, neural tissue, connective tissue, or muscle tissue) and/or those that are isolated from a common organ, tissue system, blood vessel, or other structure and/or region in an organism.

As used herein, a “combination therapy” or “administered in combination” means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In other embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination may be administered by intravenous injection while a second therapeutic agent of the combination may be administered orally.

As used herein, the terms “effective amount,” “therapeutically effective amount,” and a “sufficient amount” of a composition, antibody, vector construct, viral vector or cell described herein refer to a quantity sufficient to, when administered to a subject, including a mammal (e.g., a human), effect beneficial or desired results, including effects at the cellular level, tissue level, or clinical results, and, as such, an “effective amount” or synonym thereto depends upon the context in which it is being applied. For example, in the context of treating an inflammatory or autoimmune disease or condition it is an amount of the composition, antibody, vector construct, viral vector or cell sufficient to achieve a treatment response as compared to the response obtained without administration of the composition, antibody, vector construct, viral vector or cell. The amount of a given composition described herein that will correspond to such an amount will vary depending upon various factors, such as the given agent, the pharmaceutical formulation, the route of administration, the type of disease or disorder, the identity of the subject (e.g., age, sex, weight) or host being treated, and the like, but can nevertheless be routinely determined by one skilled in the art. Also, as used herein, a “therapeutically effective amount” of a composition, antibody, vector construct, viral vector or cell of the present disclosure is an amount that results in a beneficial or desired result in a subject as compared to a control. As defined herein, a therapeutically effective amount of a composition, antibody, vector construct, viral vector or cell of the present disclosure may be readily determined by one of ordinary skill by routine methods known in the art. Dosage regimen may be adjusted to provide the optimum therapeutic response.

As used herein, the terms “increasing” and “decreasing” refer to modulating resulting in, respectively, greater or lesser amounts, of function, expression, or activity of a metric relative to a reference. For example, subsequent to administration of an α6*nAChR activator in a method described herein, the amount of a marker of a metric (e.g., immune cell activation) as described herein may be increased or decreased in a subject by at least 5%, 10%, 15%, 20%, 25%, 30%, 35%, 40%, 45%, 50%, 55%, 60%, 65%, 70%, 75%, 80%, 85%, 90%, 95% or 98% or more relative to the amount of the marker prior to administration. Generally, the metric is measured subsequent to administration at a time that the administration has had the recited effect, e.g., at least one week, one month, 3 months, or 6 months, after a treatment regimen has begun.

As used herein, the term “innervated” refers to a tissue (e.g., a lymph node, spleen, secondary lymphoid organ, tertiary lymphoid organ, barrier tissue, skin, gut or airway) that contains nerves. “Innervation” refers to the process of nerves entering a tissue.

As used herein, “locally” or “local administration” means administration at a particular site of the body intended for a local effect and not a systemic effect. Examples of local administration are epicutaneous, inhalational, intra-articular, intrathecal, intravaginal, intravitreal, intrauterine, intra-lesional administration, lymph node administration, intratumoral administration and administration to a mucous membrane of the subject, wherein the administration is intended to have a local and not a systemic effect.

As used herein, the term “percent (%) sequence identity” refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent sequence identity (e.g., gaps can be introduced in one or both of the candidate and reference sequences for optimal alignment and non-homologous sequences can be disregarded for comparison purposes). Alignment for purposes of determining percent sequence identity can be achieved in various ways that are within the skill in the art, for instance, using publicly available computer software, such as BLAST, ALIGN, or Megalign (DNASTAR) software. Those skilled in the art can determine appropriate parameters for measuring alignment, including any algorithms needed to achieve maximal alignment over the full length of the sequences being compared. For example, a reference sequence aligned for comparison with a candidate sequence may show that the candidate sequence exhibits from 50% to 100% sequence identity across the full length of the candidate sequence or a selected portion of contiguous amino acid (or nucleic acid) residues of the candidate sequence. The length of the candidate sequence aligned for comparison purposes may be, for example, at least 30%, (e.g., 30%, 40, 50%, 60%, 70%, 80%, 90%, or 100%) of the length of the reference sequence. When a position in the candidate sequence is occupied by the same amino acid residue as the corresponding position in the reference sequence, then the molecules are identical at that position.

As used herein, a “pharmaceutical composition” or “pharmaceutical preparation” is a composition or preparation having pharmacological activity or other direct effect in the mitigation, treatment, or prevention of disease, and/or a finished dosage form or formulation thereof and which is indicated for human use.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response and other problem complications commensurate with a reasonable benefit/risk ratio.

As used herein, the term “proliferation” refers to an increase in cell numbers through growth and division of cells.

As used herein, the term “reference” refers to a level, expression level, copy number, sample or standard that is used for comparison purposes. For example, a reference sample can be obtained from a healthy individual (e.g., an individual who does not have an inflammatory or autoimmune disease or condition). A reference level can be the level of expression of one or more reference samples. For example, an average expression (e.g., a mean expression or median expression) among a plurality of individuals (e.g., healthy individuals, or individuals who do not have an inflammatory or autoimmune disease or condition). In other instances, a reference level can be a predetermined threshold level, e.g., based on functional expression as otherwise determined, e.g., by empirical assays.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and cells) isolated from a subject.

As used herein, the terms “subject” and “patient” refer to an animal (e.g., a mammal, such as a human). A subject to be treated according to the methods described herein may be one who has been diagnosed with a particular condition, or one at risk of developing such conditions. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

“Treatment” and “treating,” as used herein, refer to the medical management of a subject with the intent to improve, ameliorate, stabilize (i.e., not worsen), prevent or cure a disease, pathological condition, or disorder. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to minimizing or partially or completely inhibiting the development of the associated disease, pathological condition, or disorder); and supportive treatment (treatment employed to supplement another therapy). Treatment also includes diminishment of the extent of the disease or condition; preventing spread of the disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

As used herein, the term “under-expressed” refers to a nucleic acid or polypeptide that is expressed or caused to be expressed or produced in a cell at a lower level than is normally expressed in the corresponding wild-type cell. For example, CHRNA6 (e.g., the CHRNA6 gene or nAChRα6 protein) is “under-expressed” in an immune cell (e.g., a Treg) when CHRNA6 is present at a lower level in the immune cell compared to the level in a healthy cell of the same tissue or cell type from the same species or individual. CHRNA6 is under-expressed when CHRNA6 expression (e.g., gene or protein expression) is decreased by 1.1-fold or more (e.g., 1.1, 1.2, 1.3, 1.4, 1.5, 2.0, 2.5, 3.0, 3.5, 4.0, 4.5, 5.0, 6.0, 7.0, 8.0, 9.0, 10.0-fold or more) compared to a reference (e.g., a healthy cell of the same type).

As used herein, the term “activation” refers to the response of an immune cell to a perceived insult. When immune cells become activated, they proliferate, secrete pro-inflammatory cytokines, differentiate, present antigens, become more polarized, and become more phagocytic and cytotoxic. Factors that stimulate immune cell activation include pro-inflammatory cytokines, pathogens, and non-self antigen presentation (e.g., antigens from pathogens presented by dendritic cells, macrophages, or B cells).

As used herein, the terms “antibody-dependent cell mediated cytotoxicity” and “antibody-dependent cellular toxicity” (ADCC) refer to the killing of an antibody-coated target cell by a cytotoxic effector cell through a non-phagocytic process, characterized by the release of the content of cytotoxic granules or by the expression of cell death-inducing molecules. ADCC is triggered through interaction of target-bound antibodies (belonging to IgG or IgA or IgE classes) with certain Fc receptors (FcRs), glycoproteins present on the effector cell surface that bind the Fc region of immunoglobulins (Ig). Effector cells that mediate ADCC include natural killer (NK) cells, monocytes, macrophages, neutrophils, eosinophils and dendritic cells.

As used herein, the terms “antibody-dependent cell mediated phagocytosis” and “antibody-dependent cellular phagocytosis” (ADCP) refer to the phagocytosis (e.g., engulfment) of an antibody-coated target cell by immune cells (e.g., phagocytes). ADCP is triggered through interaction of target-bound antibodies (belonging to IgG or IgA or IgE classes) with certain Fc receptors (FcRs, e.g., FcγRIIa, FcγRIIIa, and FcγRI), glycoproteins present on the effector cell surface that bind the Fc region of immunoglobulins (Ig). Effector cells that mediate ADCP include monocytes, macrophages, neutrophils, and dendritic cells.

As used herein, the term “antigen presentation” refers to a process in which fragments of antigens are displayed on the cell surface of immune cells. Antigens are presented to T cells and B cells to stimulate an immune response. Antigen presenting cells include dendritic cells, B cells, and macrophages. Mast cells and neutrophils can also be induced to present antigens.

As used herein, the term “anti-inflammatory cytokine” refers to a cytokine produced or secreted by an immune cell that reduces inflammation. Immune cells that produce and secrete anti-inflammatory cytokines include T cells (e.g., Th cells) macrophages, B cells, and mast cells. Anti-inflammatory cytokines include IL4, IL-10, IL-11, IL-13, interferon alpha (IFNα) and transforming growth factor-beta (TGFβ).

As used herein, the term “chemokine” refers to a type of small cytokine that can induce directed chemotaxis in nearby cells. Classes of chemokines include CC chemokines, CXC chemokines, C chemokines, and CX3C chemokines. Chemokines can regulate immune cell migration and homing, including the migration and homing of monocytes, macrophages, T cells, mast cells, eosinophils, and neutrophils. Chemokines responsible for immune cell migration include CCL19, CCL21, CCL14, CCL20, CCL25, CCL27, CXCL12, CXCL13, CCR9, CCR10, and CXCR5. Chemokines that can direct the migration of inflammatory leukocytes to sites of inflammation or injury include CCL2, CCL3, CCL5, CXCL1, CXCL2, and CXCL8.

As used herein, the term “cytokine” refers to a small protein involved in cell signaling. Cytokines can be produced and secreted by immune cells, such as T cells, B cells, macrophages, and mast cells, and include chemokines, interferons, interleukins, lymphokines, and tumor necrosis factors.

As used herein, the term “cytokine production” refers to the expression, synthesis, and secretion (e.g., release) of cytokines by an immune cell.

As used herein, the term “cytotoxicity” refers to the ability of immune cells to kill other cells. Immune cells with cytotoxic functions release toxic proteins (e.g., perforin and granzymes) capable of killing nearby cells. Natural killer cells and cytotoxic T cells (e.g., CD8+ T cells) are the primary cytotoxic effector cells of the immune system, although dendritic cells, neutrophils, eosinophils, mast cells, basophils, macrophages, and monocytes have been shown to have cytotoxic activity.

As used herein, the term “differentiation” refers to the developmental process of lineage commitment. A “lineage” refers to a pathway of cellular development, in which precursor or “progenitor” cells undergo progressive physiological changes to become a specified cell type having a characteristic function (e.g., nerve cell, immune cell, or endothelial cell). Differentiation occurs in stages, whereby cells gradually become more specified until they reach full maturity, which is also referred to as “terminal differentiation.” A “terminally differentiated cell” is a cell that has committed to a specific lineage, and has reached the end stage of differentiation (i.e., a cell that has fully matured). By “committed” or “differentiated” is meant a cell that expresses one or more markers or other characteristic of a cell of a particular lineage.

As used herein, the term “degranulation” refers to a cellular process in which molecules, including antimicrobial and cytotoxic molecules, are released from intracellular secretory vesicles called granules. Degranulation is part of the immune response to pathogens and invading microorganisms by immune cells such as granulocytes (e.g., neutrophils, basophils, and eosinophils), mast cells, and lymphocytes (e.g., natural killer cells and cytotoxic T cells). The molecules released during degranulation vary by cell type and can include molecules designed to kill the invading pathogens and microorganisms or to promote an immune response, such as inflammation.

As used herein, the term “immune dysregulation” refers to a condition in which the immune system is disrupted or responding to an insult. Immune dysregulation includes aberrant activation (e.g., autoimmune disease), activation in response to an injury or disease (e.g., disease-associated inflammation), and activation in response to a pathogen or infection (e.g., parasitic infection). Immune dysregulation also includes under-activation of the immune system (e.g., immunosuppression). Immune dysregulation can be treated using the methods and compositions described herein to direct immune cells to carry out beneficial functions and reduce harmful activities (e.g., reducing activation and pro-inflammatory cytokine secretion in subjects with autoimmune disease).

As used herein, the term “modulating an immune response” refers to any alteration in a cell of the immune system or any alteration in the activity of a cell involved in the immune response. Such regulation or modulation includes an increase or decrease in the number of various cell types, an increase or decrease in the activity of these cells, or any other changes that can occur within the immune system. Cells involved in the immune response include, but are not limited to, T lymphocytes (T cells), B lymphocytes (B cells), natural killer (NK) cells, macrophages, eosinophils, mast cells, dendritic cells and neutrophils. In some cases, “modulating” the immune response means the immune response is stimulated or enhanced, and in other cases “modulating” the immune response means suppression of the immune system.

As used herein, the term “lymph node egress” refers to immune cell exit from the lymph nodes, which occurs during immune cell recirculation. Immune cells that undergo recirculation include lymphocytes (e.g., T cells, B cells, and natural killer cells), which enter the lymph node from blood to survey for antigen and then exit into lymph and return to the blood stream to perform antigen surveillance.

As used herein, the term “lymph node homing” refers to directed migration of immune cells to a lymph node. Immune cells that return to lymph nodes include T cells, B cells, macrophages, and dendritic cells.

As used herein, the term “migration” refers to the movement of immune cells throughout the body. Immune cells can migrate in response to external chemical and mechanical signals. Many immune cells circulate in blood including peripheral blood mononuclear cells (e.g., lymphocytes such as T cells, B cells, and natural killer cells), monocytes, macrophages, dendritic cells, and polymorphonuclear cells (e.g., neutrophils and eosinophils). Immune cells can migrate to sites of infection, injury, or inflammation, back to the lymph nodes, or to tumors or cancer cells.

As used herein, the term “phagocytosis” refers to the process in which a cell engulfs or ingests material, such as other cells or parts of cells (e.g., bacteria), particles, or dead or dying cells. A cell that capable of performing this function is called a phagocyte. Immune phagocytes include neutrophils, monocytes, macrophages, mast cells, B cells, eosinophils, and dendritic cells.

As used herein, the term “polarization” refers to the ability of an immune cell to shift between different functional states. A cell that is moving toward one of two functional extremes is said to be in the process of becoming more polarized. The term polarization is often used to refer to macrophages, which can shift between states known as M1 and M2. M1, or classically activated, macrophages secrete pro-inflammatory cytokines (e.g., IL-12, TNF, IL-6, IL-8, IL-1B, MCP-1, and CCL2), are highly phagocytic, and respond to pathogens and other environmental insults. M1 macrophages can also be detected by expression of Nos2. M2, or alternatively activated, macrophages secrete a different set of cytokines (e.g., IL-10) and are less phagocytic. M2 macrophages can detected by expression of Arg1, IDO, PF4, CCL24, IL10, and IL4Ra. Cells become polarized in response to external cues such as cytokines, pathogens, injury, and other signals in the tissue microenvironment.

As used herein, the term “pro-inflammatory cytokine” refers to a cytokine secreted from immune cells that promotes inflammation. Immune cells that produce and secrete pro-inflammatory cytokines include T cells (e.g., Th cells) macrophages, B cells, and mast cells. Pro-inflammatory cytokines include interleukin-1 (IL-1, e.g., IL-β3), IL-5, IL-6, IL-8, IL-10, IL-12, IL-13, IL-18, tumor necrosis factor (TNF, e.g., TNFα), interferon gamma (IFNγ), and granulocyte macrophage colony stimulating factor (GMCSF).

As used herein, the term “pro-survival cytokine” refers to a cytokine that promotes the survival of immune cells (e.g., T cells). Pro-survival cytokines include IL-2, IL-4, IL-6, IL-7, and IL-15.

As used herein, the term “recruitment” refers to the re-distribution of immune cells to a particular location (e.g., the site of infection, injury, or inflammation). Immune cells that can undergo this re-distributed and be recruited to sites of injury or disease include monocytes, macrophages, T cells, B cells, dendritic cells, and natural killer cells.

As used herein, the term “α6*nAChR-associated inflammatory or autoimmune disease or condition” refers to an inflammatory or autoimmune disease or condition that is associated with immune cells in which α6*nAChR is expressed (e.g., immune cells, such as Tregs, that express α6*nAChR or immune cells having decreased expression of α6*nAChR compared to a reference (e.g., an immune cell from a subject that does not have an inflammatory or autoimmune disease or condition)). The immune cells can be systemic immune cells or immune cells that have infiltrated the affected tissue or tissues (e.g., infiltrating immune cells or tissue resident immune cells). α6*nAChR-associated inflammatory or autoimmune diseases or conditions can be identified by assessing an immune cell or a biopsy of an immune-cell infiltrated tissue sample for immune cell nAChRα6 expression (e.g., gene or protein expression) and comparing it to nAChRα6 expression in a reference cell.

The term “α6*nAChR activating antibody” refers to antibodies that are capable of binding to an nAChR containing a nAChRα6 subunit and inducing or increasing nAChR opening or increasing or inducing nAChR activity. For example, α6*nAChR activating antibodies may promote formation of the multimeric nicotinic acetylcholine receptor complex, induce nAChR channel opening, stabilize the nAChR channel in an open state, or stimulate receptor activity. α6*nAChR activating antibodies may increase nAChR activity or channel opening by at least 10% (e.g., 10%, 20%, 30%, 40%, 50%, 60%, 70%, 80%, 90%, 95%, 98% or more).

As used herein, the term “α6*nAChR activator” refers to an agent that increases the function or activation of a nicotinic acetylcholine receptor that includes a nAChRα6 subunit. α6*nAChR activators include α6*nAChR activating antibodies and small molecule α6*nAChR activators (e.g., agonists) that induce or increase nAChR opening, stabilize the nAChR channel in an open state, or increase receptor activity. α6*nAChR activators may increase the activity of α6*nAChR by 10% or more (e.g., 10%, 20%, 30%, 40%, 50%, 60%, 70%, 80%, 90%, 95%, 98% or more).

As used herein, the terms “small molecule α6*nAChR activator” and “small molecule α6*nAChR agonist” refer to a small molecule that agonizes an α6*nAChR and has an EC50 of 10 μM or lower. A small molecule α6*nAChR agonist may bind to α6*nAChR and induce or increase channel opening, or stabilize the channel in an open conformation, allowing more ions to pass through the channel.

As used herein, the term “IFNγ-associated inflammatory or autoimmune disease or condition” refers to an inflammatory or autoimmune diseases or conditions in which IFNγ is elevated. Exemplary IFNγ-associated inflammatory or autoimmune diseases or conditions include agammaglobulinemia, autoimmune aplastic anemia, autoimmune gastric atrophy, cardiomyopathy, hemolytic anemia, lichen planus, leukocytoclastic vasculitis, linear IgA disease (LAD), lupus (SLE), multiple sclerosis, myasthenia gravis, mixed connective tissue disease (MCTD), myositis, polymyositis, psoriasis, plaque psoriasis, pure red cell aplasia, vesiculobullous dermatosis, vasculitis, and vitiligo.

As used herein, the term “inflammatory or autoimmune disease or condition associated with activated T cells” refers to an inflammatory or autoimmune diseases or conditions in which activated T cells are present. Exemplary inflammatory or autoimmune diseases or conditions associated with activated T cells include alopecia areata, autoimmune aplastic anemia, autoimmune myocarditis, autoimmune retinopathy, autoimmune thrombocytopenic purpura (ATP), celiac disease, collagen-induced arthritis, Dermatomyositis, Devic's disease, eosinophilic esophagitis, giant cell myocarditis, Evans syndrome, glomerulonephritis, and autoimmune inner ear disease.

As used herein, an agent that “does not cross the blood brain barrier” is an agent that does not significantly cross the barrier between the peripheral circulation and the brain and spinal cord. This can also be referred to as a “blood brain barrier impermeable” agent. Agents will have a limited ability to cross the blood brain barrier if they are not lipid soluble or have a molecular weight of over 600 Daltons. Agents that typically cross the blood brain barrier can be modified to become blood brain barrier impermeable based on chemical modifications that increase the size or alter the hydrophobicity of the agent, packaging modifications that reduce diffusion (e.g., packaging an agent within a microparticle or nanoparticle), and conjugation to biologics that direct the agent away from the blood brain barrier (e.g., conjugation to a pancreas-specific antibody). An agent that does not cross the blood brain barrier is an agent for which 30% or less (e.g., 30%, 25%, 20%, 15%, 10%, 5%, 2% or less) of the administered agent crosses the blood brain barrier.

As used herein, an agent that “does not have a direct effect on the central nervous system (CNS) or gut” is an agent that does not directly alter neurotransmission, neuronal numbers, or neuronal morphology in the CNS or gut when administered according to the methods described herein. This may be assessed by administering the agents to animal models and performing electrophysiological recordings or immunohistochemical analysis. An agent will be considered not to have a direct effect on the CNS or gut if administration according to the methods described herein has an effect on neurotransmission, neuronal numbers, or neuronal morphology in the CNS or gut that is 50% or less (e.g., 50%, 45%, 40%, 35%, 30%, 25%, 20%, 15%, 10%, 5%, or less) of the effect observed if the same agent is administered directly to the CNS or gut.

As used herein, the term “neuronal growth factor modulator” refers to an agent that regulates neuronal growth, development, or survival. Neuronal growth factors include proteins that promote neurogenesis, neuronal growth, and neuronal differentiation (e.g., neurotrophic factors NGF, NT3, BDNF, CNTF, and GDNF), proteins that promote neurite outgrowth (e.g., axon or dendrite outgrowth or stabilization), or proteins that promote synapse formation (e.g., synaptogenesis, synapse assembly, synaptic adhesion, synaptic maturation, synaptic refinement, or synaptic stabilization). These processes lead to innervation of tissue, including neural tissue, muscle, lymph nodes and tumors, and the formation of synaptic connections between two or more neurons and between neurons and non-neural cells (e.g., immune cells). A neuronal growth factor modulator may block one or more of these processes (e.g., through the use of antibodies that block neuronal growth factors or their receptors) or promote one or more of these processes (e.g., through the use of these proteins or analogs or peptide fragments thereof). Exemplary neuronal growth factors are listed in Table 10. Neuronal growth factor modulators decrease or increase neurite outgrowth, innervation, synapse formation, or any of the aforementioned processes by 10% or more (e.g., 10%, 20%, 30%, 40%, 50%, 60%, 70%, 80%, 90%, 95%, 98% or more).

As used herein, the term “neurotransmission modulator” refers to an agent that either induces or increases neurotransmission or decreases or blocks neurotransmission. Neurotransmission modulators can increase or decrease neurotransmission by 10%, 20%, 30%, 40%, 50%, 60%, 70%, 80%, 90%, 95%, 98% or more. Exemplary neurotransmitters and neurotransmitter receptors are listed in Tables 5 and 6. Neurotransmission modulators may increase neurotransmission by increasing neurotransmitter synthesis or release, preventing neurotransmitter reuptake or degradation, increasing neurotransmitter receptor activity, increasing neurotransmitter receptor synthesis or membrane insertion, decreasing neurotransmitter degradation, and regulating neurotransmitter receptor conformation. Neurotransmission modulators that increase neurotransmission include neurotransmitters and analogs thereof and neurotransmitter receptor agonists. Neurotransmission modulators may decrease neurotransmission by decreasing neurotransmitter synthesis or release, increasing neurotransmitter reuptake or degradation, decreasing neurotransmitter receptor activity, decreasing neurotransmitter receptor synthesis or membrane insertion, increasing neurotransmitter degradation, regulating neurotransmitter receptor conformation, and disrupting the pre- or postsynaptic machinery. Neurotransmission modulators that decrease or block neurotransmission include antibodies that bind to or block the function of neurotransmitters, neurotransmitter receptor antagonists, and toxins that disrupt synaptic release.

### DETAILED DESCRIPTION

Described herein are compositions and methods for the treatment of an inflammatory or autoimmune disease or condition in a subject (e.g., a mammalian subject, such as a human) by administering α6*nAChR activators. α6*nAChR activators include α6*nAChR activating antibodies and small molecule α6*nAChR activators (e.g., agonists). These methods and compositions provide new mechanistic approaches for treating inflammatory or autoimmune diseases or conditions.

α6*nAChR

Cholinergic receptor nicotinic alpha 6 subunit (CHRNA6, Entrez Gene 8973) encodes the alpha-6 subunit (nAChRα6) of the nicotinic acetylcholine receptor (nAChR). The nicotinic acetylcholine receptor is made up of five subunits, arranged symmetrically around a central pore. There are various assemblies of receptors, either homomeric (all one type of subunit) or heteromeric (at least one α and one β) combinations of twelve different nicotinic receptor subunits: α1-α10, β1-β4, delta, gamma, and epsilon. The subunits are categorized by sequence homology into four families. nAChRα6 is a member of family III subtype 1, along with nAChRα2, nAChRα3, and nAChRα4. After binding acetylcholine, the nAChR responds by an extensive change in conformation that affects all subunits and leads to the opening of an ion-conducting channel across the plasma membrane.

nAChRα6 subunits are known to be included in nAChRβ2-subunit containing nAChRs, and nicotinic acetylcholine receptors containing α6 and β2 subunits are thought to play a role in nicotine addiction. nAChRs containing α6 and β2 subunits are enriched in the dorsal and ventral striatum of the brain and are also expressed by retinal ganglion cells and in catacholaminergic and retinal projection regions of the brain. Within the brain, α6 and β2-containing nAChRs have also been found to include β3, and α4 subunits, and the two major α6 and β2-subunit containing nAChRs expressed in the brain are thought to be α4α6β2β3nAChRs and α6β2β3nAChRs.

The present invention relates to the discovery that, contrary to the conventional wisdom that nAChRα6 is a neuronal nAChR subunit, nAChRα6 is highly and specifically expressed in regulatory T cells (Tregs). These findings indicate that α6*nAChR activators can be added to Tregs to induce Treg activation to treat autoimmune diseases devoid of Tregs usually present in steady state barrier tissue or to protect the host from excessive, aberrant immune responses. Through this mechanism, activation of nAChRs containing a nAChRα6 subunit can reduce inflammation, induce tolerance, and be used as a therapeutic strategy for treating Treg-mediated inflammatory and autoimmune diseases or conditions.

α6*nAChR Activators

α6*nAChR activators described herein can activate α6*nAChRs in order to treat an inflammatory or autoimmune disease or condition. The activators may activate α6*nAChRs by binding to an α6*nAChR and inducing or increasing channel opening or stabilizing the channel in an open state.

In some embodiments, the α6*nAChR activator is an α6*nAChR activating antibody or an antigen binding fragment thereof that increases or induces receptor activity. α6*nAChR activating antibodies include antibodies that agonize (e.g., increase activity) α6*nAChRs, such as by increasing or inducing channel opening or stabilizing the channel in an open conformation. These antibodies may bind directly to nAChRα6 or to nAChRα6 and/or other subunits that are known to be expressed in α6*nAChR, such as nAChRβ2. Antibodies having one or more of these functional properties are routinely screened and selected once the desired functional property is identified herein (e.g., by screening of phage display or other antibody libraries).

In some embodiments, the α6*nAChR activator is a small molecule α6*nAChR activator (e.g., agonist). Small molecule α6*nAChR agonists for use in the methods and compositions described herein are provided Table 1.

TABLE 1
SMALL MOLECULE α6*nAChR ACTIVATORS

| Target | Activators |
|---|---|
| nAChRs containing | Nicotine; 5-iodo-3-(2(S)-azetidinylmethoxy)pyridine; 9-methyl-3-pyridin-3-yl-3,9- |
| a nAChRα6 subunit | diaza-bicyclo[3.3.1]nonane; varenicline; carbachol; epibatidine; ispronicline; |
| | AZD1446; ABT-894; ABT-089; ABT-560; NS9283; RJR-2403; TC 2429 |
| | (described in Drenan et al., Neuron 60:123-136,2008, structure shown below) |
| | 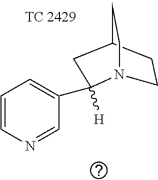 [SMILES: c1cncc(C2CC3CCN2CC3)c1] |

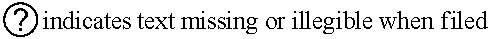

### Agent Modalities

An α6*nAChR activator can be selected from a number of different modalities. An α6*nAChR activator can be a small molecule (e.g., a small molecule activator (e.g., an agonist), or a polypeptide (e.g., an antibody or antigen binding fragment thereof). An α6*nAChR activator can also be a viral vector expressing an α6*nAChR activator or a cell infected with a viral vector. Any of these modalities can be an α6*nAChR activator directed to target (e.g., to induce or increase) the activity of an nAChR containing a nAChRα6 subunit.

The small molecule or antibody molecule can be modified. For example, the modification can be a chemical modification, e.g., conjugation to a marker, e.g., fluorescent marker or a radioactive marker. In other examples, the modification can include conjugation to a molecule that enhances the stability or half-life of the α6*nAChR activator (e.g., an Fc domain of an antibody or serum albumin, e.g., human serum albumin). The modification can also include conjugation to an antibody to target the agent to a particular cell or tissue. Additionally, the modification can be a chemical modification, packaging modification (e.g., packaging within a nanoparticle or microparticle), or targeting modification to prevent the agent from crossing the blood brain barrier.

Small Molecules

Numerous small molecule α6*nAChR activators (e.g., agonists) useful in the methods of the invention are described herein and additional small molecule α6*nAChR activators useful as therapies for inflammatory or autoimmune diseases or conditions can also be identified through screening based on their ability to bind to α6*nAChR and induce or increase channel opening and/or activity. Small molecules include, but are not limited to, small peptides, peptidomimetics (e.g., peptoids), amino acids, amino acid analogs, synthetic polynucleotides, polynucleotide analogs, nucleotides, nucleotide analogs, organic and inorganic compounds (including heterorganic and organometallic compounds) generally having a molecular weight less than about 5,000 grams per mole, e.g., organic or inorganic compounds having a molecular weight less than about 2,000 grams per mole, e.g., organic or inorganic compounds having a molecular weight less than about 1,000 grams per mole, e.g., organic or inorganic compounds having a molecular weight less than about 500 grams per mole, and salts, esters, and other pharmaceutically acceptable forms of such compounds.

In some embodiments, the small molecule α6*nAChR agonist is a small molecule listed in Table 1. Small molecule α6*nAChR agonists can be used to treat a disorder or condition described herein. A pharmaceutical composition including the small molecule α6*nAChR agonist can be formulated for treatment of an inflammatory or autoimmune disease or condition described herein. In some embodiments, a pharmaceutical composition that includes the small molecule α6*nAChR agonist is formulated for local administration, e.g., to the affected site in a subject.

Antibodies

The α6*nAChR activator can be an antibody or antigen binding fragment thereof (e.g., an agonist antibody). For example, an α6*nAChR activator described herein is an α6*nAChR activating antibody that increases or promotes the activity of the receptor through binding to the receptor and stabilizing it in an open conformation.

The making and use of therapeutic antibodies against a target antigen (e.g., against α6*nAChR) is known in the art. See, for example, the references cited herein above, as well as Zhiqiang An (Editor), Therapeutic Monoclonal Antibodies: From Bench to Clinic. 1st Edition. Wiley 2009, and also Greenfield (Ed.), Antibodies: A Laboratory Manual. (Second edition) Cold Spring Harbor Laboratory Press 2013, for methods of making recombinant antibodies, including antibody engineering, use of degenerate oligonucleotides, 5′-RACE, phage display, and mutagenesis; antibody testing and characterization; antibody pharmacokinetics and pharmacodynamics; antibody purification and storage; and screening and labeling techniques.

Viral Vectors

Viral vectors can be used to express a neurotoxin from Table 9 as a combination therapy with an α6*nAChR activator. A viral vector expressing a neurotoxin from Table 9 can be administered to a cell or to a subject (e.g., a human subject or animal model) to decrease or block neurotransmission. Viral vectors can be directly administered (e.g., injected) to a lymph node, spleen, gut, barrier tissue, or airway to treat an inflammatory or autoimmune disease or condition.

Viral genomes provide a rich source of vectors that can be used for the efficient delivery of exogenous genes into a mammalian cell. Viral genomes are particularly useful vectors for gene delivery because the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors include a retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus, replication deficient herpes virus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses include: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology (Third Edition) Lippincott-Raven, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in U.S. Pat. No. 5,801,030, the teachings of which are incorporated herein by reference.

Cell-Based Therapies

An α6*nAChR activator described herein can be administered to a cell in vitro (e.g., an immune cell), which can subsequently be administered to a subject (e.g., a human subject or animal model). The α6*nAChR activator can be administered to the cell to effect an immune response (e.g., activation, polarization, antigen presentation, cytokine production, migration, proliferation, or differentiation) as described herein. Once the immune response is elicited, the cell can be administered to a subject (e.g., injected) to treat an autoimmune or inflammatory disease or condition. The immune cell can be locally administered (e.g., injected into a lymph node, spleen, secondary lymphoid organ, tertiary lymphoid organ, barrier tissue, skin, gut or airway).

The cell can be administered to a subject immediately after being contacted with an α6*nAChR activator (e.g., within 5, 10, 15, 30, 45, or 60 minutes of being contacted with an α6*nAChR activator), or 6 hours, 12 hours, 24 hours, 2 days, 3, days, 4 days, 5, days, 6 days, 7 days or more after being contacted with an α6*nAChR activator. The method can include an additional step of evaluating the immune cell for an immune cell activity (e.g., activation, polarization, antigen presentation, cytokine production, migration, proliferation, or differentiation) or modulation of gene expression after contact with an α6*nAChR activator and before administration to a subject.

Blood Brain Barrier Permeability

In some embodiments, the α6*nAChR activators α6*nAChR activators for use in the present invention are agents that are not capable of crossing, or that do not cross, the blood brain barrier (BBB) of a mammal, e.g., an experimental rodent (e.g., mouse or rat), dog, pig, non-human primate, or a human. The BBB is a highly selective semipermeable membrane barrier that separates the circulating blood from the brain extracellular fluid (e.g., cerebrospinal fluid) in the central nervous system (CNS). The BBB is made up of high-density endothelial cells, which are connected by tight junctions. These cells prevent most molecular compounds in the bloodstream (e.g., large molecules and hydrophilic molecules) from entering the brain. Water, some gases (e.g., oxygen and carbon dioxide), and lipid-soluble molecules (e.g., hydrophobic molecules, such as steroid hormones) can cross the BBB by passive diffusion. Molecules that are needed for neural function, such as glucose and amino acids, are actively transported across the BBB.

A number of approaches can be used to render an agent BBB impermeable. These methods include modifications to increase an agent's size, polarity, or flexibility or reduce its lipophilicity, targeting approaches to direct an agent to another part of the body and away from the brain, and packaging approaches to deliver an agent in a form that does not freely diffuse across the BBB. These approaches can be used to render a BBB permeable α6*nAChR activator impermeable, and they can also be used to improve the properties (e.g., cell-specific targeting) of an α6*nAChR activator that does not cross the BBB. The methods that can be used to render an agent BBB impermeable are discussed in greater detail herein below.

Formulation of BBB-Impermeable Agents for Enhanced Cell Targeting

One approach that can be used to render an α6*nAChR activator BBB impermeable is to conjugate the agent to a targeting moiety that directs it somewhere other than the brain. The targeting moiety can be an antibody for a receptor expressed by the target cell (e.g., N-Acetylgalactosamine for liver transport; DGCR2, GBF1, GPR44 or SerpinB10 for pancreas transport; Secretoglobin, family 1A, member 1 for lung transport). The targeting moiety can also be a ligand of any receptor or other molecular identifier expressed on the target cell in the periphery. These targeting moieties can direct the α6*nAChR activator of interest to its corresponding target cell, and can also prevent BBB crossing by directing the agent away from the BBB and increasing the size of the α6*nAChR activator via conjugation of the targeting moiety.

Activators of nAChRs containing a nAChRα6 subunit can also be rendered BBB impermeable through formulation in a particulate delivery system (e.g., a nanoparticle, liposome, or microparticle), such that the agent is not freely diffusible in blood and cannot cross the BBB. The particulate formulation used can be chosen based on the desired localization of the α6*nAChR activator (e.g., a lymph node, lymphoid organ, or site of inflammation), as particles of different sizes accumulate in different locations. For example, nanoparticles with a diameter of 45 nm or less enter the lymph node, while 100 nm nanoparticles exhibit poor lymph node trafficking. Some examples of the link between particle size and localization in vivo are described in Reddy et al., J Controlled Release 112:26 2006, and Reddy et al., Nature Biotechnology 25:1159 2007.

Activators of nAChRs containing a nAChRα6 subunit can be tested after the addition of a targeting moiety or after formulation in a particulate delivery system to determine whether or not they cross the BBB. Models for assessing BBB permeability include in vitro models (e.g., monolayer models, co-culture models, dynamic models, multi-fluidic models, isolated brain microvessels), in vivo models, and computational models as described in He et al., Stroke 45:2514 2014; Bickel, NeuroRx 2:15 2005; and Wang et al., Int J Pharm 288:349 2005. An α6*nAChR activator that exhibits BBB impermeability can be used in the methods described herein.

Modification of Existing Compounds to Render them BBB Impermeable

There are multiple parameters that have been empirically derived in the field of medicinal chemistry to predict whether a compound will cross the BBB. The most common numeric value for describing permeability across the BBB is the logBB, defined as the logarithmic ratio of the concentration of a compound in the brain and in the blood. Empirical rules of thumb have been developed to predict BBB permeability, including rules regarding molecular size, polar surface area, sum of oxygen and nitrogen atoms, lipophilicity (e.g., partition coefficient between apolar solvent and water), “lipoaffinity”, molecular flexibility, and number of rotatable bonds (summarized in Muehlbacher et al., J Comput Aided Mol Des. 25: 1095 2011; and Geldenhuys et al., Ther Deliv. 6: 961 2015). Some preferred limits on various parameters for BBB permeability are listed in Table 1 of Ghose et al., ACS Chem Neurosci. 3: 50 2012, which is incorporated herein by reference. Based on the parameters shown in the table, one of skill in the art could modify an existing α6*nAChR activator to render it BBB impermeable.

One method of modifying an α6*nAChR activator to prevent BBB crossing is to add a molecular adduct that does not affect the target binding specificity, kinetics, or thermodynamics of the agent. Molecular adducts that can be used to render an agent BBB impermeable include polyethylene glycol (PEG), a carbohydrate monomer or polymer, a dendrimer, a polypeptide, a charged ion, a hydrophilic group, deuterium, and fluorine. α6*nAChR can be tested after the addition of one or more molecular adducts or after any other properties are altered to determine whether or not they cross the BBB. Models for assessing BBB permeability include in vitro models (e.g., monolayer models, co-culture models, dynamic models, multi-fluidic models, isolated brain microvessels), in vivo models, and computational models as described in He et al., Stroke 45:2514 2014; Bickel, NeuroRx 2:15 2005; and Wang et al., Int J Pharm 288:349 2005. An α6*nAChR activator that exhibits BBB impermeability can be used in the methods described herein.

Screening for or Development of BBB Impermeable Agents

Another option for developing BBB impermeable agents is to find or develop new agents that do not cross the BBB. One method for finding new BBB impermeable agents is to screen for compounds that are BBB impermeable. Compound screening can be performed using in vitro models (e.g., monolayer models, co-culture models, dynamic models, multi-fluidic models, isolated brain microvessels), in vivo models, and computational models, as described in He et al., Stroke 45:2514 2014; Bickel, NeuroRx 2:15 2005; Wang et al., Int J Pharm 288:349 2005, and Czupalla et al., Methods Mol Biol 1135:415 2014. For example, the ability of a molecule to cross the blood brain barrier can be determined in vitro using a transwell BBB assay in which microvascular endothelial cells and pericytes are co-cultured separated by a thin macroporous membrane, see e.g., Naik et al., J Pharm Sci 101:1337 2012 and Hanada et al., Int J Mol Sci 15:1812 2014; or in vivo by tracking the brain uptake of the target molecule by histology or radio-detection. Compounds would be deemed appropriate for use as α6*nAChR activators α6*nAChR activators in the methods described herein if they do not display BBB permeability in the aforementioned models.

Modulation of Immune Cells

The methods described herein can be used to modulate an immune response in a subject or cell by administering to a subject or cell an α6*nAChR activator in a dose (e.g., an effective amount) and for a time sufficient to modulate the immune response. These methods can be used to treat a subject in need of modulating an immune response, e.g., a subject with an inflammatory condition, an autoimmune disease or condition. One way to modulate an immune response is to modulate an immune cell activity. This modulation can occur in vivo (e.g., in a human subject or animal model) or in vitro (e.g., in acutely isolated or cultured cells, such as human cells from a patient, repository, or cell line, or rodent cells). The types of cells that can be modulated include T cells (e.g., peripheral T cells, cytotoxic T cells/CD8+ T cells, T helper cells/CD4+ T cells, memory T cells, regulatory T cells/Tregs, natural killer T cells/NKTs, mucosal associated invariant T cells, and gamma delta T cells), B cells (e.g., memory B cells, plasmablasts, plasma cells, follicular B cells/B-2 cells, marginal zone B cells, B-1 cells, regulatory B cells/Bregs), dendritic cells (e.g., myeloid DCs/conventional DCs, plasmacytoid DCs, or follicular DCs), granulocytes (e.g., eosinophils, mast cells, neutrophils, and basophils), monocytes, macrophages (e.g., peripheral macrophages or tissue resident macrophages), myeloid-derived suppressor cells, natural killer (NK) cells, innate lymphoid cells (ILC1, ILC2, ILC3), thymocytes, and megakaryocytes.

The immune cell activities that can be modulated by administering to a subject or contacting a cell with an effective amount of an α6*nAChR activator described herein include activation (e.g., macrophage, T cell, NK cell, ILC, B cell, dendritic cell, neutrophil, eosinophil, or basophil activation), phagocytosis (e.g., macrophage, neutrophil, monocyte, mast cell, B cell, eosinophil, or dendritic cell phagocytosis), antibody-dependent cell-mediated phagocytosis (e.g., ADCP by monocytes, macrophages, neutrophils, or dendritic cells), antibody-dependent cell-mediated cytotoxicity (e.g., ADCC by NK cells, ILCs, monocytes, macrophages, neutrophils, eosinophils, dendritic cells, or T cells), polarization (e.g., macrophage polarization toward an M1 or M2 phenotype or T cell polarization), proliferation (e.g., proliferation of B cells, T cells, monocytes, macrophages, dendritic cells, NK cells, ILCs, mast cells, neutrophils, eosinophils, or basophils), lymph node homing (e.g., lymph node homing of T cells, B cells, dendritic cells, or macrophages), lymph node egress (e.g., lymph node egress of T cells, B cells, dendritic cells, or macrophages), recruitment (e.g., recruitment of B cells, T cells, monocytes, macrophages, dendritic cells, NK cells, ILCs, mast cells, neutrophils, eosinophils, or basophils), migration (e.g., migration of B cells, T cells, monocytes, macrophages, dendritic cells, NK cells, ILCs, mast cells, neutrophils, eosinophils, or basophils), differentiation (e.g., regulatory T cell differentiation), immune cell cytokine production, antigen presentation (e.g., dendritic cell, macrophage, and B cell antigen presentation), maturation (e.g., dendritic cell maturation), and degranulation (e.g., mast cell, NK cell, ILC, cytotoxic T cell, neutrophil, eosinophil, or basophil degranulation). Innervation of lymph nodes or lymphoid organs, development of high endothelial venules (HEVs), and development of ectopic or tertiary lymphoid organs (TLOs) can also be modulated using the methods described herein. Modulation can increase or decrease these activities, depending on the α6*nAChR activator used to contact the cell or treat a subject.

In some embodiments, an effective amount of an α6*nAChR activator is an amount sufficient to modulate (e.g., increase or decrease) one or more (e.g., 2 or more, 3 or more, 4 or more) of the following immune cell activities in the subject or cell: T cell polarization; T cell activation; dendritic cell activation; neutrophil activation; eosinophil activation; basophil activation; T cell proliferation; B cell proliferation; T cell proliferation; monocyte proliferation; macrophage proliferation; dendritic cell proliferation; NK cell proliferation; mast cell proliferation; ILC proliferation; neutrophil proliferation; eosinophil proliferation; basophil proliferation; cytotoxic T cell activation; circulating monocytes; peripheral blood hematopoietic stem cells; macrophage polarization; macrophage phagocytosis; macrophage ADCP, neutrophil phagocytosis; monocyte phagocytosis; mast cell phagocytosis; B cell phagocytosis; eosinophil phagocytosis; dendritic cell phagocytosis; macrophage activation; antigen presentation (e.g., dendritic cell, macrophage, and B cell antigen presentation); antigen presenting cell migration (e.g., dendritic cell, macrophage, and B cell migration); lymph node immune cell homing and cell egress (e.g., lymph node homing and egress of T cells, B cells, dendritic cells, or macrophages); NK cell activation; NK cell ADCC, mast cell degranulation; NK cell degranulation; ILC activation; ILC ADCC, ILC degranulation; cytotoxic T cell degranulation; neutrophil degranulation; eosinophil degranulation; basophil degranulation; neutrophil recruitment; eosinophil recruitment; NKT cell activation; B cell activation; regulatory T cell differentiation; dendritic cell maturation; development of HEVs; development of TLOs; or lymph node or secondary lymphoid organ innervation. In certain embodiments, the immune response (e.g., an immune cell activity listed herein) is increased or decreased in the subject or cell at least 1%, 2%, 5%, 10%, 15%, 20%, 25%, 30%, 35%, 40%, 50%, 60%, 70%, 80%, 100%, 150%, 200%, 300%, 400%, 500% or more, compared to before the administration. In certain embodiments, the immune response is increased or decreased in the subject or cell between 5-20%, between 5-50%, between 10-50%, between 20-80%, between 20-70%, between 50-200%, between 100%-500%.

After an α6*nAChR activator is administered to treat a patient or contact a cell, a readout can be used to assess the effect on immune cell activity. Immune cell activity can be assessed by measuring a cytokine or marker associated with a particular immune cell type, as listed in Table 2 (e.g., performing an assay listed in Table 2 for the cytokine or marker). In certain embodiments, the parameter is increased or decreased in the subject at least 1%, 2%, 5%, 10%, 15%, 20%, 25%, 30%, 35%, 40%, 50%, 60%, 70%, 80%, 100%, 150%, 200%, 300%, 400%, 500% or more, compared to before the administration. In certain embodiments, the parameter is increased or decreased in the subject between 5-20%, between 5-50%, between 10-50%, between 20-80%, between 20-70%, between 50-200%, between 100%-500%. An α6*nAChR activator can be administered at a dose (e.g., an effective amount) and for a time sufficient to modulate an immune cell activity described herein below.

After an α6*nAChR activator is administered to treat a patient or contact a cell, a readout can be used to assess the effect on immune cell migration. Immune cell migration can be assessed by measuring the number of immune cells in a location of interest (e.g., lymph node, spleen, secondary lymphoid organ, tertiary lymphoid organ, barrier tissue, skin, gut or airway). Immune cell migration can also be assessed by measuring a chemokine, receptor, or marker associated with immune cell migration, as listed in Tables 3 and 4. In certain embodiments, the parameter is increased or decreased in the subject at least 1%, 2%, 5%, 10%, 15%, 20%, 25%, 30%, 35%, 40%, 50%, 60%, 70%, 80%, 100%, 150%, 200%, 300%, 400%, 500% or more, compared to before the administration. In certain embodiments, the parameter is increased or decreased in the subject between 5-20%, between 5-50%, between 10-50%, between 20-80%, between 20-70%, between 50-200%, between 100%-500%. An α6*nAChR activator can be administered at a dose (e.g., an effective amount) and for a time sufficient to modulate an immune cell migration as described herein below.

An α6*nAChR activator described herein can affect immune cell migration. Immune cell migration between peripheral tissues, the blood, and the lymphatic system as well as lymphoid organs is essential for the orchestration of productive innate and adaptive immune responses. Immune cell migration is largely regulated by trafficking molecules including integrins, immunoglobulin cell-adhesion molecules (IgSF CAMs), cadherins, selectins, and a family of small cytokines called chemokines (Table 3). Cell adhesion molecules and chemokines regulate immune cell migration by both inducing extravasation from the circulation into peripheral tissues and acting as guidance cues within peripheral tissues themselves. For extravasation to occur, chemokines must act in concert with multiple trafficking molecules including C-type lectins (L-, P-, and E-selectin), multiple integrins, and cell adhesion molecules (ICAM-1, VCAM-1 and MAdCAM-1) to enable a multi-step cascade of immune cell capturing, rolling, arrest, and transmigration via the blood endothelial barrier (Table 4). Some trafficking molecules are constitutively expressed and manage the migration of immune cells during homeostasis, while others are specifically upregulated by inflammatory processes such as infection and autoimmunity.

The expression of trafficking molecules important for extravasation is mainly regulated on specialized blood vessels called HEVs, which are the entry portals from the circulation into the periphery and are usually present in secondary lymphoid organs (SLOs) and chronically inflamed tissue. Chronically inflamed tissues often develop lymphoid-like structures called TLOs that contain structures resembling SLOs including HEVs, lymphoid stromal cells, and confined compartments of T and B lymphocytes. As they can act as major gateways for immune cell migration into peripheral tissues, TLOs have been shown to be important in the pathogenesis of autoimmune disorders.

Once within peripheral tissues, four modes of immune cell migration have been observed: 1) chemokinesis: migration driven by soluble chemokines, without concentration gradients to provide directional bias, 2) haptokinesis: migration along surfaces presenting immobilized ligands such as chemokines or integrins, without concentration gradients to provide directional bias, 3) chemotaxis: directional migration driven by concentration gradients of soluble chemokines, and 4) haptotaxis: directional migration along surfaces presenting gradients of immobilized ligands such as chemokines or integrins. The response of immune cells to trafficking molecules present on the endothelium depends on the composition, expression, and/or functional activity of their cognate receptors, which in turn depends on activation state and immune cell subtype.

Innate immune cells generally migrate toward inflammation-induced trafficking molecules in the periphery. In contrast, naïve T and B cells constantly re-circulate between the blood and secondary lymphoid organs to screen for their cognate antigen presented by activated dendritic cells (DCs) or fibroblastic reticular cells (FRCs), respectively. If activated by recognition of their cognate antigen and appropriate co-stimulation within SLOs, both cell types undergo a series of complex maturation steps, including differentiation and proliferation, ultimately leading to effector and memory immune cell phenotypes. To reach their peripheral target sites, certain effector and memory T and B cell subsets egress from SLOs to the blood circulation via efferent lymphatics. In order to do so, they migrate toward a Sphingosine-1-phosphate (S1P) gradient sensed using their Sphingosine-1-phosphate receptor 1 (S1P₁ or S1PR1). For successful egress into efferent lymphatics, immune cells need to overcome SLO retention signals through the CCR7/CCL21 axis or through CD69-mediated downregulation of S1P₁.

Finally, certain immune cell subsets, for example mature dendritic cells (DCs) and memory T cells, migrate from peripheral tissues into SLOs via afferent lymphatics. To exit from peripheral tissues and enter afferent lymphatics, immune cells again largely depend on the CCR7/CCL21 and S1P₁/S1P axis. Specifically, immune cells need to overcome retention signals delivered via the CCR7/CCL21 axis, and migrate toward an S1P gradient established by the lymphatic endothelial cells using S1P₁. The selective action of trafficking molecules on distinct immune cell subsets as well as the distinct spatial and temporal expression patterns of both the ligands and receptors are crucial for the fine-tuning of immune responses during homeostasis and disease.

Aberrant immune cell migration is observed in multiple immune-related pathologies. Immune cell adhesion deficiencies, caused by molecular defects in integrin expression, fucosylation of selectin ligands, or inside-out activation of integrins on leukocytes and platelets, lead to impaired immune cell migration into peripheral tissues. This results in leukocytosis and in increased susceptibility to recurrent bacterial and fungal infections, which can be difficult to treat and potentially life-threatening. Alternatively, exaggerated migration of specific immune cell subsets into specific peripheral tissues is associated with a multitude of pathologies. For example, excessive neutrophil accumulation in peripheral tissues contributes to the development of ischemia-reperfusion injury, such as that observed during acute myocardial infarction, stroke, shock and acute respiratory distress syndrome. Excessive Th1 inflammation characterized by tissue infiltration of interferon-gamma secreting effector T cells and activated macrophages is associated with atherosclerosis, allograft rejection, hepatitis, and multiple autoimmune diseases including multiple sclerosis, rheumatoid arthritis, psoriasis, Crohn's disease, type 1 diabetes and lupus erythematodes. Excessive Th2 inflammation characterized by tissue infiltration of IL-4, IL-5, and IL-13 secreting Th2 cells, eosinophils and mast cells is associated with asthma, food allergies and atopic dermatitis.

In some embodiments, an α6*nAChR activator described herein increases one or more of Treg migration, Treg proliferation, Treg recruitment, Treg activation, Treg polarization, or Treg cytokine production. In some embodiments, the cytokine is an anti-inflammatory cytokine (e.g., IL10 and/or transforming growth factor beta (TGFβ)). In some embodiments, the α6*nAChR activator described herein increases Treg expression of α6*nAChR.

In some embodiments, the effect of the α6*nAChR activator on Tregs has a secondary effect on pro-inflammatory immune cells, such as CD8+ T cells, CD4+ T cells, NK cells, macrophages and dendritic cells. In some embodiments, the effect of the α6*nAChR activator on Tregs leads to a decrease in pro-inflammatory immune cell migration, proliferation, recruitment, activation, polarization, cytokine production, (e.g., a decrease in production of pro-inflammatory cytokines), ADCC, or ADCP. In some embodiments, the effect of the α6*nAChR activator on Tregs leads to a decrease in T cell (e.g., CD8+ T cell) activation. In some embodiments, the effect of the α6*nAChR activator on Tregs leads to a decrease in T cell (e.g., CD8+ T cell) pro-inflammatory cytokine production (e.g., IFNγ production).

Immune Effects

A variety of in vitro and in vivo assays can be used to determine how an α6*nAChR activator affects an immune cell activity. The effect of an α6*nAChR activator on T cell polarization in a subject can be assessed by evaluation of cell surface markers on T cells obtained from the subject. A blood sample, lymph node biopsy, or tissue sample can be collected from a subject and T cells from the sample evaluated for one or more (e.g., 2, 3, or 4 or more) Th1-specific markers: T-bet, IL-12R, STAT4, or chemokine receptors CCR5, CXCR6, and CXCR3; or Th2-specific markers: CCR3, CXCR4, or IL-4Ra. T cell polarization can also be assessed using the same methods in an in vivo animal model. This assay can also be performed by adding an α6*nAChR activator to T cells in vitro (e.g., T cells obtained from a subject, animal model, repository, or commercial source) and measuring the aforementioned markers to evaluate T cell polarization. These markers can be assessed using flow cytometry, immunohistochemistry, in situ hybridization, and other assays that allow for measurement of cellular markers. Comparing results from before and after administration of an α6*nAChR activator can be used to determine its effect.

The effect of an α6*nAChR activator on T cell activation in a subject can be assessed by evaluation of cellular markers on T cells obtained from the subject. A blood sample, lymph node biopsy, or tissue sample can be collected from a subject and T cells from the sample evaluated for one or more (e.g., 2, 3, 4 or more) activation markers: CD25, CD71, CD26, CD27, CD28, CD30, CD154, CD40L, CD134, CD69, CD62L or CD44. T cell activation can also be assessed using the same methods in an in vivo animal model. This assay can also be performed by adding an α6*nAChR activator to T cells in vitro (e.g., T cells obtained from a subject, animal model, repository, or commercial source) and measuring the aforementioned markers to evaluate T cell activation. Similar approaches can be used to assess the effect of an α6*nAChR activator on activation of other immune cells, such as eosinophils (markers: CD35, CD11b, CD66, CD69 and CD81), dendritic cells (makers: IL-8, MHC class II, CD40, CD80, CD83, and CD86), basophils (CD63, CD13, CD4, and CD203c), and neutrophils (CD11b, CD35, CD66b and CD63). These markers can be assessed using flow cytometry, immunohistochemistry, in situ hybridization, and other assays that allow for measurement of cellular markers. Comparing results from before and after administration of an α6*nAChR activator can be used to determine its effect.

The effect of an α6*nAChR activator on immune cell activation can also be assessed through measurement of secreted cytokines and chemokines. An activated immune cell (e.g., T cell, B cell, macrophage, monocyte, dendritic cell, eosinophil, basophil, mast cell, NK cell, or neutrophil) can produce pro-inflammatory cytokines and chemokines (e.g., IL-β3, IL-5, IL-6, IL-8, IL-10, IL-12, IL-13, IL-18, TNFα, and IFN-γ). Activation can be assessed by measuring cytokine levels in a blood sample, lymph node biopsy, or tissue sample from a human subject or animal model, with higher levels of pro-inflammatory cytokines following treatment with an α6*nAChR activator indicating increased activation, and lower levels indicating decreased activation. Activation can also be assessed in vitro by measuring cytokines secreted into the media by cultured cells. Cytokines can be measured using ELISA, western blot analysis, and other approaches for quantifying secreted proteins. Comparing results from before and after administration of an α6*nAChR activator can be used to determine its effect.

The effect of an α6*nAChR activator on T cell proliferation in a subject can be assessed by evaluation of markers of proliferation in T cells obtained from the subject. A blood sample, lymph node biopsy, or tissue sample can be collected from a subject and T cells from the sample evaluated for Ki67 marker expression. T cell proliferation can also be assessed using the same methods in an in vivo animal model. This assay can also be performed by adding an α6*nAChR activator to T cells in vitro (e.g., T cells obtained from a subject, animal model, repository, or commercial source) and measuring Ki67 to evaluate T cell proliferation. Assessing whether an α6*nAChR activator induces T cell proliferation can also be performed by in vivo (e.g., in a human subject or animal model) by collecting blood samples before and after administration of an α6*nAChR activator and comparing T cell numbers, and in vitro by quantifying T cell numbers before and after contacting T cells with an α6*nAChR activator. These approaches can also be used to measure the effect of an α6*nAChR activator on proliferation of any immune cell (e.g., B cells, T cells, macrophages, monocytes, dendritic cells, NK cells, mast cells, eosinophils, basophils, and neutrophils). Ki67 can be assessed using flow cytometry, immunohistochemistry, in situ hybridization, and other assays that allow for measurement of nuclear markers. Comparing results from before and after administration of an α6*nAChR activator can be used to determine its effect.

The effect of an α6*nAChR activator on cytotoxic T cell activation in a subject can be assessed by evaluation of T cell granule markers in T cells obtained from the subject. A blood sample, lymph node biopsy, or tissue sample can be collected from a subject and T cells from the sample evaluated for granzyme or perforin expression. Cytotoxic T cell activation can also be assessed using the same methods in an in vivo animal model. This assay can also be performed by adding an α6*nAChR activator to cytotoxic T cells in vitro (e.g., cytotoxic T cells obtained from a subject, animal model, repository, or commercial source) and measuring the aforementioned markers to evaluate T cell proliferation. These markers can be detected in the media from cytotoxic T cell cultures. Techniques including ELISA, western blot analysis can be used to detect granzyme and perforin in conditioned media, flow cytometry, immunohistochemistry, in situ hybridization, and other assays can detect intracellular granzyme and perforin and their synthesis. Comparing results from before and after administration of an α6*nAChR activator can be used to determine its effect.

The effect of an α6*nAChR activator on circulating monocytes in a subject can be assessed by evaluation of cell surface markers on primary blood mononuclear cells obtained from the subject. A blood sample, lymph node biopsy, or tissue sample can be collected from a subject and monocytes from the sample evaluated for CD14 and/or CD16 expression. Circulating monocytes can also be assessed using the same methods in an in vivo animal model. This assay can be performed by taking a blood sample before treatment with an α6*nAChR activator and comparing it to a blood sample taken after treatment. CD14 and CD16 can be detected using flow cytometry, immunohistochemistry, western blot analysis, or any other technique that can measure cell surface protein levels. Comparing results from before and after administration of an α6*nAChR activator can be used to determine its effect. This assay can be used to detect the number of monocytes in the bloodstream or to determine whether monocytes have adopted a CD14+/CD16+ phenotype, which indicates a pro-inflammatory function.

The effect of an α6*nAChR activator on peripheral blood hematopoietic stem cells in a subject can be assessed by evaluation of cell surface markers on primary blood mononuclear cells obtained from the subject. A blood sample, lymph node biopsy, or tissue sample can be collected from a subject and stem cells from the sample evaluated for one or more (2, 3 or 4 or more) specific markers: CD34, c-kit, Sca-1, or Thy1.1. Peripheral blood hematopoietic stem cells can also be assessed using the same methods in an in vivo animal model. This assay can be performed by taking a blood sample before treatment with an α6*nAChR activator and comparing it to a blood sample taken after treatment. The aforementioned markers can be detected using flow cytometry, immunohistochemistry, western blot analysis, or any other technique that can measure cell surface protein levels. Comparing results from before and after administration of an α6*nAChR activator can be used to determine its effect. This assay can be used to detect the number of stem cells mobilized into the bloodstream or to determine whether treatment induces differentiation into a particular hematopoietic lineage (e.g., decreased CD34 and increased GPA indicates differentiation into red blood cells, decreased CD34 and increased CD14 indicates differentiation into monocytes, decreased CD34 and increased CD11 b or CD68 indicates differentiation into macrophages, decreased CD34 and increased CD42b indicates differentiation into platelets, decreased CD34 and increased CD3 indicates differentiation into T cells, decreased CD34 and increased CD19 indicates differentiation into B cells, decreased CD34 and increased CD25 or CD69 indicates differentiation into activated T cells, decreased CD34 and increased CD1c, CD83, CD141, CD209, or MHC II indicates differentiation into dendritic cells, decreased CD34 and increased CD56 indicates differentiation into NK cells, decreased CD34 and increased CD15 indicates differentiation into neutrophils, decreased CD34 and increased 2D7 antigen, CD123, or CD203c indicates differentiation into basophils, and decreased CD34 and increased CD193, EMR1, or Siglec-8 indicates differentiation into eosinophils.

The effect of an α6*nAChR activator on macrophage polarization in a subject can be assessed by evaluation of cellular markers in macrophages cells obtained from the subject. A blood sample, lymph node biopsy, or tissue sample can be collected from a subject and macrophages from the sample evaluated for one of more (2, 3 or 4 or more) specific markers. Markers for M1 polarization include IL-12, TNF, IL-β3, IL-6, IL-23, MARCO, MHC-II, CD86, iNOS, CXCL9, and CXCL10. Markers for M2 polarized macrophages include IL-10, IL1-RA, TGFβ, MR, CD163, DC-SIGN, Dectin-1, HO-1, arginase (Arg-1), CCL17, CCL22 and CCL24. Macrophage polarization can also be assessed using the same methods in an in vivo animal model. This assay can also be performed on cultured macrophages obtained from a subject, an animal model, repository, or commercial source to determine how contacting a macrophage with an α6*nAChR activator affects polarization. The aforementioned markers can be evaluated by comparing measurements obtained before and after administration of an α6*nAChR activator to a subject, animal model, or cultured cell. Surface markers or intracellular proteins (e.g., MHC-11, CD86, iNOS, CD163, Dectin-1, HO-1, Arg-1, etc.) can be measured using flow cytometry, immunohistochemistry, in situ hybridization, or western blot analysis, and secreted proteins (e.g., IL-12, TNF, IL-β3, IL-10, TGFβ, IL1-RA, chemokines CXC8, CXC9, CCL17, CCL22, and CCL24, etc.) can be measured using the same methods or by ELISA or western blot analysis of culture media or blood samples. Comparing results from before and after administration of an α6*nAChR activator can be used to determine its effect.

The effect of an α6*nAChR activator on macrophage phagocytosis in a subject can be assessed by culturing macrophages obtained from the subject with fluorescent beads. A blood sample, lymph node biopsy, or tissue sample can be collected from a subject and macrophages from the sample evaluated for engulfment of fluorescent beads. This assay can also be performed on cultured macrophages obtained from an animal model, repository, or commercial source to determine how contacting a macrophage with an α6*nAChR activator affects phagocytosis. The same phagocytosis assay can be used to evaluate the effect of an α6*nAChR activator on phagocytosis in other immune cells (e.g., neutrophils, monocytes, mast cells, B cells, eosinophils, or dendritic cells). Comparing results from before and after administration of an α6*nAChR activator can be used to determine its effect on phagocytosis.

In some embodiments, phagocytosis is ADCP. ADCP can be assessed using similar methods to those described above by incubating immune cells (e.g., macrophages, neutrophils, monocytes, mast cells, B cells, eosinophils, or dendritic cells) isolated from a blood sample, lymph node biopsy, or tissue sample with fluorescent beads coated with IgG antibodies. In some embodiments, immune cells are incubated with a target cell line that has been pre-coated with antibodies to a surface antigen expressed by the target cell line. ADCP can be evaluated by measuring fluorescence inside the immune cell or quantifying the number of beads or cells engulfed. This assay can also be performed on cultured immune cells obtained from an animal model, repository, or commercial source to determine how contacting an immune cell with an α6*nAChR activator affects ADCP. The ability of an immune cell to perform ADCP can also be evaluated by assessing expression of certain Fc receptors (e.g., FcγRIIa, FcγRIIIa, and FcγRI). Fc receptor expression can be assessed using flow cytometry, immunohistochemistry, in situ hybridization, or other assays that allow for measurement of cell surface markers. Comparing phagocytosis or Fc receptor expression before and after administration of an α6*nAChR activator can be used to determine its effect on ACDP. In some embodiments, the α6*nAChR activator decreases macrophage ADCP of auto-antibody coated cells (e.g., in autoimmune diseases such as glomerular nephritis).

The effect of an α6*nAChR activator on macrophage activation in a subject can be assessed by evaluation of cell surface markers on macrophages cells obtained from the subject. A blood sample, lymph node biopsy, or tissue sample can be collected from a subject and macrophages from the sample evaluated for one or more (e.g., 1, 2, 3 or 4 or more) specific markers: F4/80, HLA molecules (e.g., MHC-II), CD80, CD68, CD11b, or CD86. Macrophage activation can also be assessed using the same methods in an in vivo animal model. This assay can also be performed by adding an α6*nAChR activator to macrophages in vitro (e.g., macrophages obtained from a subject, animal model, repository, or commercial source) and measuring the aforementioned markers to evaluate macrophage activation. These markers can be assessed using flow cytometry, immunohistochemistry, in situ hybridization, and other assays that allow for measurement of cell surface markers. As mentioned above, macrophage activation can also be evaluated based on cytokine production (e.g., pro-inflammatory cytokine production) as measured by ELISA and western blot analysis. Comparing results from before and after administration of an α6*nAChR activator can be used to determine its effect.

The effect of an α6*nAChR activator on antigen presentation in a subject can be assessed by evaluation of cell surface markers on antigen presenting cells (e.g., dendritic cells, macrophages, and B cells) obtained from the subject. A blood sample, lymph node biopsy, or tissue sample can be collected from a subject and antigen presenting cells (e.g., dendritic cells, macrophages, and B cells) from the sample evaluated for one or more (e.g., 2, 3 or 4 or more) specific markers: CD11c, CD11b, HLA molecules (e.g., MHC-II), CD40, B7, IL-2, CD80 or CD86. Antigen presentation can also be assessed using the same methods in an in vivo animal model. This assay can also be performed by adding an α6*nAChR activator to antigen presenting cells (e.g., dendritic cells) in vitro (e.g., antigen presenting cells obtained from a subject, animal model, repository, or commercial source) and measuring the aforementioned markers to evaluate antigen presentation. These markers can be assessed using flow cytometry, immunohistochemistry, in situ hybridization, and other assays that allow for measurement of cell surface markers. Comparing results from before and after administration of an α6*nAChR activator can be used to determine its effect.

The effect of an α6*nAChR activator on antigen presenting cell migration in a subject can be assessed by evaluation of cell surface markers on antigen presenting cells (e.g., dendritic cells, B cells, and macrophages) obtained from the subject. A blood sample, lymph node biopsy, or tissue sample can be collected from a subject and antigen presenting cells (e.g., dendritic cells, B cells, and macrophages) from the sample evaluated for CCR7 expression. Antigen presenting cell migration can also be assessed using the same methods in an in vivo animal model. This assay can also be performed by adding an α6*nAChR activator to antigen presenting cells (e.g., dendritic cells, B cells, and macrophages) in vitro (e.g., antigen presenting cells obtained from a subject, animal model, repository, or commercial source) and measuring CCR7 to evaluate antigen presenting cell migration. CCR7 can be assessed using flow cytometry, immunohistochemistry, in situ hybridization, and other assays that allow for measurement of cell surface markers. Comparing results from before and after administration of an α6*nAChR activator can be used to determine its effect.

The effect of an α6*nAChR activator on lymph node immune cell homing and cell egress in a subject can be assessed by evaluation of cell surface markers on T or B cells obtained from the subject. A blood sample, lymph node biopsy, or tissue sample can be collected from a subject and T or B cells from the sample evaluated for one or more specific markers: CCR7 or S1PR1. Lymph node immune cell homing and cell egress can also be assessed using the same methods in an in vivo animal model. This assay can also be performed by adding an α6*nAChR activator to T or B cells in vitro (e.g., T or B cells obtained from a subject, animal model, repository, or commercial source) and measuring the aforementioned markers to evaluate T or B cell lymph node homing. These markers can also be used to assess lymph node homing and cell egress of dendritic cells and macrophages. CCR7 and S1PR1 can be assessed using flow cytometry, immunohistochemistry, in situ hybridization, and other assays that allow for measurement of cell surface markers. If using an animal model, lymph nodes or sites of inflammation can be imaged in vivo (e.g., using a mouse that expresses fluorescently labeled T or B cells) or after biopsy to determine whether T or B cell numbers change as a result of administration of an α6*nAChR activator. Comparing results from before and after administration of an α6*nAChR activator can be used to determine its effect.

In some embodiments, an α6*nAChR activator increases homing or decreases egress of naïve T cells into or out of secondary lymphoid organs prior to antigen challenge (e.g., prior to administration of a vaccine) to generate a better antigen-specific response. In some embodiments, an α6*nAChR activator decreases homing or increases egress of inflammatory immune cells (e.g., neutrophils) into or out of peripheral tissues during acute infection or injury to prevent conditions such as ischemia-reperfusion disorders. In some embodiments, an α6*nAChR activator decreases homing or increases egress of effector immune subsets into or out of peripheral tissues to avoid inflammation-induced tissue damage in autoimmune diseases.

The effect of an α6*nAChR activator on NK cell activation in a subject can be assessed by evaluation of cell surface markers on NK cells obtained from the subject. A blood sample, lymph node biopsy, or tissue sample can be collected from a subject and NK cells from the sample evaluated for one or more (e.g., 2, 3 or 4 or more) specific markers: CD117, NKp46, CD94, CD56, CD16, KIR, CD69, HLA-DR, CD38, KLRG1, and TIA-1. NK cell activation can also be assessed using the same methods in an in vivo animal model. This assay can also be performed by adding an α6*nAChR activator to NK cells in vitro (e.g., NK cells obtained from a subject, animal model, repository, or commercial source) and measuring the aforementioned markers to evaluate NK cell activation. The effect of an α6*nAChR activator can be determined by comparing results from before and after administration of the α6*nAChR activator.

In some embodiments, activated NK cells have increased lytic function or are cytotoxic (e.g., capable of performing ADCC). The effect of an α6*nAChR activator on ADCC can be assessed by incubating immune cells capable of ADCC (e.g., NK cells, monocytes, macrophages, neutrophils, eosinophils, dendritic cells, or T cells) with a target cell line that has been pre-coated with antibodies to a surface antigen expressed by the target cell line. ADCC can be assessed by measuring the number of surviving target cells with a fluorescent viability stain or by measuring the secretion of cytolytic granules (e.g., perforin, granzymes, or other cytolytic proteins released from immune cells). Immune cells can be collected from a blood sample, lymph node biopsy, or tissue sample from a human subject or animal model treated with an α6*nAChR activator. This assay can also be performed by adding an α6*nAChR activator to immune cells in vitro (e.g., immune cells obtained from a subject, animal model, repository, or commercial source). The effect of an α6*nAChR activator on ADCC can be determined by comparing results from before and after administration of the α6*nAChR activator.

The effect of an α6*nAChR activator on mast cell degranulation in a subject can be assessed by evaluation of markers in mast cells obtained from the subject. A blood sample, lymph node biopsy, or tissue sample can be collected from a subject and mast cells from the sample evaluated for one or more (e.g., 1, 2, 3 or 4 or more) specific markers: IgE, histamine, IL-4, TNFα, CD300a, tryptase, or MMP9. Mast cell degranulation can also be assessed using the same methods in an in vivo animal model. This assay can also be performed by adding an α6*nAChR activator to mast cells in vitro (e.g., mast cells obtained from a subject, animal model, repository, or commercial source) and measuring the aforementioned markers to evaluate mast cell degranulation. Some of these markers (e.g., histamine, TNFα, and IL-4) can be detected by measuring levels in the mast cell culture medium after mast cells are contacted with an α6*nAChR activator. The effect of an α6*nAChR activator can be determined by comparing results from before and after administration of the α6*nAChR activator. This approach can also be used to evaluate the effect of an α6*nAChR activator on degranulation by other cells, such as neutrophils (markers: CD11 b, CD13, CD18, CD45, CD15, CD66b IL-β3, IL-8, and IL-6), eosinophils (markers: major basic protein (MBP), eosinophil cationic protein (ECP), eosinophil peroxidase (EPX), eosinophil-derived neurotoxin (EDN)), basophils (markers: histamine, heparin, chondroitin, elastase, lysophospholipase, and LTD-4), NK cells (markers: LAMP-1, perforin, and granzymes), and cytotoxic T cells (markers: LAMP-1, perforin, and granzymes). Markers can be detected using flow cytometry, immunohistochemistry, ELISA, western blot analysis, or in situ hybridization.

The effect of an α6*nAChR activator on neutrophil recruitment in a subject can be assessed by evaluation of cell surface markers on neutrophils obtained from the subject. A blood sample, lymph node biopsy, or tissue sample can be collected from a subject and neutrophils from the sample evaluated for one or more (e.g., 1, 2, 3 or 4 or more) specific markers: CD11b, CD14, CD114, CD177, CD354, or CD66. To determine whether neutrophils are being recruited to a specific site (e.g., a site of inflammation), the same markers can be measured at the site of inflammation. Neutrophil recruitment can also be assessed using the same methods in an in vivo animal model. This assay can also be performed by adding an α6*nAChR activator to neutrophils in vitro (e.g., neutrophils obtained from a subject, animal model, repository, or commercial source) and measuring the aforementioned markers to evaluate neutrophil recruitment. These markers can be assessed using flow cytometry, immunohistochemistry, in situ hybridization, and other assays that allow for measurement of cell surface markers. The effect of an α6*nAChR activator can be determined by comparing results from before and after administration of the α6*nAChR activator.

The effect of an α6*nAChR activator on eosinophil recruitment in a subject can be assessed by evaluation of cell surface markers on eosinophil obtained from the subject. A blood sample, lymph node biopsy, or tissue sample can be collected from a subject and eosinophils from the sample evaluated for one or more (e.g., 1, 2, 3 or 4 or more) specific markers: CD15, IL-3R, CD38, CD106, CD294 or CD85G. To determine whether eosinophils are being recruited to a specific site (e.g., a site of inflammation), the same markers can be measured at the site of inflammation. Eosinophil recruitment can also be assessed using the same methods in an in vivo animal model. This assay can also be performed by adding an α6*nAChR activator to eosinophils in vitro (e.g., eosinophils obtained from a subject, animal model, repository, or commercial source) and measuring the aforementioned markers to evaluate eosinophil recruitment. These markers can be assessed using flow cytometry, immunohistochemistry, in situ hybridization, and other assays that allow for measurement of cell surface markers. The effect of an α6*nAChR activator can be determined by comparing results from before and after administration of the α6*nAChR activator.

The effect of an α6*nAChR activator on NKT cell activation in a subject can be assessed by evaluation of cell surface markers on NKT cells obtained from the subject. A blood sample, lymph node biopsy, or tissue sample can be collected from a subject and NKT cells from the sample evaluated for one or more specific markers: CD272 or CD352. Activated NKT cells produce IFN-γ, IL-4, GM-CSF, IL-2, IL-13, IL-17, IL-21 and TNFα. NKT cell activation can also be assessed using the same methods in an in vivo animal model. This assay can also be performed by adding an α6*nAChR activator to NKT cells in vitro (e.g., NKT cells obtained from a subject, animal model, repository, or commercial source) and measuring the aforementioned markers to evaluate NKT cell activation. Cell surface markers CD272 and CD352 can be assessed using flow cytometry, immunohistochemistry, in situ hybridization, and other assays that allow for measurement of cell surface markers. The secreted proteins can be detected in blood samples or cell culture media using ELISA, western blot analysis, or other methods for detecting proteins in solution. The effect of an α6*nAChR activator can be determined by comparing results from before and after administration of the α6*nAChR activator.

The effects of an α6*nAChR activator on B cell activation in a subject can be assessed by evaluation of cell surface markers on B cells obtained from the subject. A blood sample, lymph node biopsy, or tissue sample can be collected from a subject and B cells from the sample evaluated for one or more (e.g., 2, 3 or 4 or more) specific markers: CD19, CD20, CD40, CD80, CD86, CD69, IgM, IgD, IgG, IgE, or IgA. B cell activation can also be assessed using the same methods in an in vivo animal model. This assay can also be performed by adding an α6*nAChR activator to B cells in vitro (e.g., B cells obtained from a subject, animal model, repository, or commercial source) and measuring the aforementioned markers to evaluate B cell activation. These markers can be assessed using flow cytometry, immunohistochemistry, in situ hybridization, and other assays that allow for measurement of cell surface markers. The effect of an α6*nAChR activator can be determined by comparing results from before and after administration of the α6*nAChR activator.

The effect of an α6*nAChR activator on regulatory T cell differentiation in a subject can be assessed by evaluation of markers in regulatory T cells obtained from the subject. A blood sample, lymph node biopsy, or tissue sample can be collected from a subject and regulatory T cells from the sample evaluated for one or more (e.g., 1, 2, 3, 4 or more) specific markers: CD4, CD25, or FoxP3. Regulatory T cell differentiation can also be assessed using the same methods in an in vivo animal model. This assay can also be performed by adding an α6*nAChR activator to regulatory T cells in vitro (e.g., regulatory T cells obtained from a subject, animal model, repository, or commercial source) and measuring the aforementioned markers to evaluate regulatory T cell differentiation. These markers can be assessed using flow cytometry, immunohistochemistry, in situ hybridization, and other assays that allow for measurement of cellular markers. The effect of an α6*nAChR activator can be determined by comparing results from before and after administration of the α6*nAChR activator.

The effect of an α6*nAChR activator on innervation of a lymph node or secondary lymphoid organ can be assessed by evaluation of neuronal markers in a lymph node or secondary lymphoid organ biopsy sample obtained from a human subject or animal model. A biopsy can be collected from the subject and evaluated for one or more (e.g., 1, 2, 3, 4, or 4 or more) neuronal markers selected from: Neurofilament, synapsin, synaptotagmin, or neuron specific enolase. Lymph node innervation can also be assessed using electrophysiological approaches (e.g., recording neuronal activity in a lymph node or secondary lymphoid organ in a human subject or animal model). The effect of an α6*nAChR activator can be determined by comparing results from before and after administration of the α6*nAChR activator.

The α6*nAChR activator can also reduce the number of nerve fibers in the affected tissue or reduce the activity of peripheral nerve fibers in the affected tissue. For example, the method includes administering to the subject (e.g., a human subject or animal model) an α6*nAChR activator in an amount and for a time sufficient to reduce the number of nerve fibers in the affected tissue or reduce the activity of peripheral nerve fibers in the affected tissue. The affected tissue can be a lymph node, a lymphoid organ, or the bone marrow niche. The number of nerve fibers in the affected tissue or the activity of peripheral nerve fibers in the affected tissue can be decreased in the subject at least 1%, 2%, 5%, 10%, 15%, 20%, 25%, 30%, 35%, 40%, 50%, 60%, 70%, 80%, 90%, 95% or more, compared to before the administration. The number of nerve fibers in the affected tissue or the activity of peripheral nerve fibers in the affected tissue can be decreased in the subject between 5-20%, between 5-50%, between 10-50%, between 20-80%, between 20-70%.

The α6*nAChR activator can also increase the number of nerve fibers in the affected tissue or increase the activity of peripheral nerve fibers in the affected tissue. For example, the method includes administering to the subject (e.g., a human subject or animal model) an α6*nAChR activator in an amount and for a time sufficient to increase the number of nerve fibers in the affected tissue or increase the activity of peripheral nerve fibers in the affected tissue. The affected tissue can be a lymph node, a lymphoid organ, or the bone marrow niche. The number of nerve fibers in the affected tissue or the activity of peripheral nerve fibers in the affected tissue can be increased in the subject at least 1%, 2%, 5%, 10%, 15%, 20%, 25%, 30%, 35%, 40%, 50%, 60%, 70%, 80% or more, compared to before the administration. The number of nerve fibers in the affected tissue or the activity of peripheral nerve fibers in the affected tissue can be increased in the subject between 5-20%, between 5-50%, between 10-50%, between 20-80%, between 20-70%.

The nerve fibers that are modulated can be part of the peripheral nervous system, e.g., a somatic nerve, an autonomic nerve, a sensory nerve, a cranial nerve, an optic nerve, an olfactory nerve, a sympathetic nerve, a parasympathetic nerve, a chemoreceptor, a photoreceptor, a mechanoreceptor, a thermoreceptor, a nociceptor, an efferent nerve fiber, or an afferent nerve fiber.

The effect of an α6*nAChR activator on immune cell cytokine production can be assessed by evaluation of cellular markers in an immune cell sample obtained from a human subject or animal model. A blood sample, lymph node biopsy, or tissue sample can be collected for the subject and evaluated for one or more (e.g., 1, 2, 3, 4, or 4 or more) cytokine markers selected from: pro-inflammatory cytokines (e.g., IL-β3, IL-5, IL-6, IL-8, IL-10, IL-12, IL-13, IL-18, TNFα, IFNγ, GMCSF), pro-survival cytokines (e.g., IL-2, IL-4, IL-6, IL-7, and IL-15) and anti-inflammatory cytokines (e.g., IL-4, IL-10, IL-11, IL-13, IFNα, and TGFβ). Some cytokines can function as both pro- and anti-inflammatory cytokines depending on context or indication (e.g., IL-4 is often categorized as an anti-inflammatory cytokine, but plays a pro-inflammatory role in mounting an allergic or anti-parasitic immune response). Cytokines can be also detected in the culture media of immune cells contacted with an α6*nAChR activator. Cytokines can be detected using ELISA, western blot analysis, or other methods for detecting protein levels in solution. The effect of an α6*nAChR activator can be determined by comparing results from before and after administration of the α6*nAChR activator.

In some embodiments, an α6*nAChR activator decreases or prevents the development of TLOs to decrease local inflammation in autoimmune diseases. TLOs are highly similar to SLOs and exhibit T and B cell compartmentalization, APCs such as DCs and follicular DCs, stromal cells, and a highly organized vascular system of high endothelial venules. In some embodiments, an α6*nAChR activator decreases or prevents the development of HEVs within tertiary lymphoid organs to decrease local inflammation in autoimmune diseases. HEVs can be detected using the monoclonal antibody MECA-79.

In some embodiments, an α6*nAChR activator modulates dendritic cell maturation (e.g., activation). Dendritic cell maturation can be increased to promote their migration from peripheral tissues into secondary lymphoid organs to improve T cell activation in the draining lymph node (e.g., to increase vaccine efficacy or to improve immune defense against infectious agents). Dendritic cell maturation can be decreased to decrease their migration from peripheral tissues into secondary lymphoid organs to inhibit T cell activation in the draining lymph node (e.g., to improve outcomes in organ transplantation or to reduce the severity of or treat autoimmune diseases).

Table 2 lists additional markers and relevant assays that may be used to assess the level, function and/or activity of immune cells in the methods described herein.

TABLE 2
ASSESSMENT OF IMMUNE CELL PHENOTYPES

| | ASSOCIATED | | |
|---|---|---|---|
| IMMUNE CELL | CYTOKINES | MARKER | ASSAYS |
| Th1 helper | IFN-γ | CD4 | ELISPOT |
| | IL-2 | CD94 | In situ hybridization |
| | IL-12 | CD119 | Immunohistochemistry |
| | IL-18 | (IFNγ R1) | Limiting dilution Analysis |
| | IL-27 | CD183 | Single-cell PCR |
| | TNFα | (CXCR3) | In vivo capture assay |
| | TNFβ/LTα | CD186 | ELISA |
| | | (CXCR6) | Flow cytometry |
| | | CD191 | |
| | | (CCR1) | |
| | | CD195 | |
| | | (CCR5) | |
| | | CD212 (IL- | |
| | | 12Rβ1&2) | |
| | | CD254 | |
| | | (RANKL) | |
| | | CD278 | |
| | | (ICOS) | |
| | | IL-18R | |
| | | MRP1 | |
| | | NOTCH3 | |
| | | TCR | |
| | | TIM3 | |
| Th2 helper | IL-4 | CD4 | ELISPOT |
| | IL-2 | CD30 | In situ hybridization |
| | IL-6 | CD119 | Immunohistochemistry |
| | IL-33 | (IFNγ R1) | Limiting dilution |
| | IL-17E (IL-25) | CD184 | Analysis |
| | IL-31 | (CXCR4) | Single-cell PCR |
| | IL-3 | CD185 | In vivo capture |
| | IL-10 | (CXCR5) | assay |
| | IL-13 | CD193 | ELISA |
| | | (CCR3) | Flow cytometry |
| | | CD194 | |
| | | (CCR4) | |
| | | CD197 | |
| | | (CCR7) | |
| | | CD278 | |
| | | (ICOS) | |
| | | CD294 | |
| | | (CRTh2) | |
| | | CDw198 | |
| | | (CCR8) | |
| | | IL-17RB | |
| | | IL-33Rα | |
| | | (ST2) | |
| | | NOTCH1 | |
| | | NOTCH2 | |
| | | TCR | |
| | | TIM1 | |
| Th17 helper | TGFβ1 | CD4 | ELISPOT |
| | IL-1β | CD27 | In situ hybridization |
| | IL-6 | CD62L | Immunohistochemistry |
| | IL-21 | CD127 (IL- | Limiting dilution |
| | IL-23 | 7R) | Analysis |
| | IL-17A | CD161 | Single-cell PCR |
| | IL-17F | CD184 | In vivo capture |
| | IL-22 | (CXCR4) | assay |
| | IL-26 | CD194 | ELISA |
| | GM-CSF | (CCR4) | Flow cytometry |
| | MIP-3α | CD196 | |
| | TNFα | (CCR6) | |
| | | CD197 | |
| | | (CCR7) | |
| | | CD212b1 | |
| | | (IL-12Rβ1) | |
| | | CD213a1 | |
| | | (IL-13Rα1) | |
| | | CD278 | |
| | | (ICOS) | |
| | | IL-1R1 | |
| | | IL-21R | |
| | | IL-23R | |
| Treg | TGFβ1 | CD4 | ELISPOT |
| | IL-2 | CD25 | In situ hybridization |
| | IL-10 | CD39 | Immunohistochemistry |
| | IL-35 | CD73 | Limiting dilution |
| | | CD45RO | Analysis |
| | | CD121a (IL- | Single-cell PCR |
| | | 1R1) | In vivo capture |
| | | CD121b (IL- | assay |
| | | 1R2) | ELISA |
| | | CD127low | Flow cytometry |
| | | CD134 | |
| | | (OX40) | |
| | | CD137 (4- | |
| | | 1BB) | |
| | | CD152 | |
| | | (CTLA-4) | |
| | | CD357 | |
| | | (GITR/AITR) | |
| | | Foxp3 | |
| | | FR4 (m) | |
| | | GARP | |
| | | (activated) | |
| | | Helios | |
| | | LAP/TGFβ | |
| | | (activated) | |
| | | TIGIT | |
| Dendritic cell | GM-CSF | CD1a | ELISPOT |
| | IFNγ | CD8 | In situ hybridization |
| | IL-4 | CD11c | Immunohistochemistry |
| | GM-CSF | CD80 | Limiting dilution |
| | IFNα | CD83 | Analysis |
| | IL-1α | CD85 (ILT) family | Single-cell PCR |
| | IL-1β | CD86 | In vivo capture |
| | IL-6 | CD141 (h) | assay |
| | IL-8 | CD169 | ELISA |
| | IL-10 | CD172 | Flow cytometry |
| | IL-12 | CD184 (CXCR4) | |
| | IL-15 | CD197 (CCR7) | |
| | IL-18 | CD205 | |
| | IL-23 | CD206 | |
| | IL-27 | CD207 | |
| | IP-10 | CD209 | |
| | M-CSF | CD215 (IL-15R) | |
| | RANTES (CCL5) | CD282 (TLR2) | |
| | TGFβ | CD284 (TLR4) | |
| | TNFα | CD286 (TLR6) | |
| | | Clec Family | |
| Macrophages/ | FLT3 Ligand | CD11b | ELISPOT |
| Monocytes | GM-CSF | CD14 (mono) | In situ hybridization |
| | M-CSF | CD16 | Immunohistochemistry |
| | CXCL9 | CD32 | Limiting dilution |
| | CXCL10 | CD68 | Analysis |
| | CXCL11 | CD85a (ILT5) | Single-cell PCR |
| | G-CSF | CD163 | In vivo capture |
| | GM-CSF | CD169 | assay |
| | IFNβ | CD195 (CCR5) | ELISA |
| | IL-1α | CD204 | Flow cytometry |
| | IL-1β | CD206 | |
| | IL-6 | CD282 (TLR2) | |
| | IL-8 | CD284 (TLR4) | |
| | IL-10 | CD286 (TLR6) | |
| | IL-12p40 & p70 | CD354 (Trem-1) | |
| | IL-18 | Clec Family | |
| | IL-23 | F4/80 (m) | |
| | IL-27 | HLA-DR | |
| | M-CSF | | |
| | MIP-2α (CXCL2) | | |
| | RANTES (CCL5) | | |
| | TNFα | | |
| Natural Killer Cell | IL-2 | CD16 | ELISPOT |
| | IL-12 | CD25 | In situ hybridization |
| | IL-15/IL-15R | CD49b | Immunohistochemistry |
| | IL-18 | CD56 (h) | Limiting dilution |
| | Granzyme B | CD94 | Analysis |
| | IL-17A | CD158 family (KIR) | Single-cell PCR |
| | IL-22 | (h) | In vivo capture |
| | MIP-1α (CCL3) | CD181 (CXCR1) | assay |
| | MIP-1β (CCL4) | CD183 (CXCR3) | ELISA |
| | Perforin | CD184 (CXCR4) | Flow cytometry |
| | RANTES (CCL5) | CD186 (CXCR6) | |
| | TNFα | CD192 (activated) | |
| | | CD195 (CCR5) | |
| | | CD197 (CCR7) | |
| | | CD212 (IL-12R) | |
| | | CD244 | |
| | | CD314 (NKG2D) | |
| | | CX3CR1 | |
| | | Eomes | |
| | | KLRG1 | |
| | | Ly49 family (m) | |
| | | NK1.1 | |
| | | NKG2A | |
| | | NKp30, NKp42 | |
| | | NKp44 (h), NKp46 | |
| | | T-bet | |
| Innate Lymphoid | IFN-γ | CD335 (NKp46) | ELISPOT |
| Cell 1 (ILC1) | TNF | CD336 (NKp44) | In situ hybridization |
| | | CD94 | Immunohistochemistry |
| | | CD56 (NCAM) | Limiting dilution |
| | | CD103 | Analysis |
| | | T-bet | Single-cell PCR |
| | | | In vivo capture |
| | | | assay |
| | | | ELISA |
| | | | Flow cytometry |
| Innate Lymphoid | Areg | CD127 | ELISPOT |
| Cell 2 (ILC2) | IL-5 | CRTH2 | In situ hybridization |
| | IL-13 | ST2 (IL-33R) | Immunohistochemistry |
| | | RORα | Limiting dilution |
| | | GATA3 | Analysis |
| | | | Single-cell PCR |
| | | | In vivo capture |
| | | | assay |
| | | | ELISA |
| | | | Flow cytometry |
| Innate Lymphoid | CCL3 | CD127 | ELISPOT |
| Cell 3 (ILC3) | LTs | CD117 (c-kit) | In situ hybridization |
| | IL-22 | CD335 (NKp46) | Immunohistochemistry |
| | IL-17 | CD336 (NKp44) | Limiting dilution |
| | IFN-γ | IL-23R | Analysis |
| | | RORγt | Single-cell PCR |
| | | | In vivo capture |
| | | | assay |
| | | | ELISA |
| | | | Flow cytometry |
| Activated B | Antibodies | CD19 | Flow cytometry |
| cell/Plasma cells | IgM | CD25 | |
| | IgG | CD30 | |
| | IgD | IgM | |
| | IgE | CD19 | |
| | IgA | IgG | |
| | | CD27 | |
| | | CD38 | |
| | | CD78 | |
| | | CD138 | |
| | | CD319 | |

TABLE 3
EXAMPLES OF HUMAN CHEMOKINES

| | | | | | |
|---|---|---|---|---|---|
| Systematic | Human | Alternate | | Human receptor(s) and | Known |
| name | gene | human names | Expression | their expression | functions |

C Family

| | | | | | |
|---|---|---|---|---|---|
| XCL1 | XCL1 | Lymphotactin, | activated CD8+ T | XCR1: cross-presenting | migration and |
| | | SCM-1 alpha, | cells and other | drendritic cells | activation of |
| | | ATAC | MHCI restricted T | | lymphocytes, |
| | | | cells | | NK cells |
| XCL2 | XCL2 | SCM-1 beta | expressed in | XCR1: cross-presenting | migration and |
| | | | activated T cells | drendritic cells | activation of |
| | | | | | lymphocytes, |
| | | | | | NK cells |

CX3C Family

| | | | | | |
|---|---|---|---|---|---|
| CX3CL1 | CX3CL1 | Fractalkine, | brain, heart, lung, | CX3CR1: lymphocytes, | migration and |
| | | Neurotactin, | kidney, skeletal | monocytes | adhesion of |
| | | ABCD-3 | muscle and testis. | | lymphocytes |
| | | | Up-regulated in | | and monocytes |
| | | | endothelial cells and | | |
| | | | microglia by | | |
| | | | inflammation | | |

CC Family

| | | | | | |
|---|---|---|---|---|---|
| CCL1 | CCL1 | I-309 | activated T cells | CCR8: natural killer | migration of |
| | | | | cells, monocytes and | monocytes, NK |
| | | | | lymphocytes | cells, immature |
| | | | | DARC: erytrocytes, | B cells and |
| | | | | endothelial and epithelial | DCs |
| | | | | cells | |
| CCL2 | CCL2 | MCP-1, | monocytes, | CCR2: monocytes | migration of |
| | | MCAF, HC11 | macrophages and | CCR4: lymphocytes | monocytes and |
| | | | dendritic cells, | CCR11: unkown | basophils |
| | | | activated NK cells | D6: lymphocytes, | |
| | | | | lymphatic endothelial | |
| | | | | cells, macrophages | |
| | | | | DARC: erytrocytes, | |
| | | | | endothelial and epithelial | |
| | | | | cells | |
| CCL3 | CCL3 | MIP-1 alpha, | T cells, B cells, and | CCR1: lymphocytes, | adhesion of |
| | | LD78 alpha, | monocytes after | monocytes, airway | lymphocytes |
| | | GOS19, | antigen or mitogen | smooth muscle cells | |
| | | Pat464 | stimulation | CCR4: lymphocytes | |
| | | | | CCR5: T cells, | |
| | | | | macrophages, dendritic | |
| | | | | cells, eosinophils and | |
| | | | | microglia | |
| | | | | D6: lymphocytes, | |
| | | | | lymphatic endothelial | |
| | | | | cells, macrophages | |
| CCL3L1 | CCL3L1 | LD78 beta | Unknown | CCR1: lymphocytes, | migration of |
| | | | | monocytes, airway | lymphocytes |
| | | | | smooth muscle cells | and monocytes |
| | | | | CCR3: eosinophils, | |
| | | | | basophils, Th2 cells, | |
| | | | | CD34+ hematopoetic | |
| | | | | progenitors, | |
| | | | | keratinocytes, mast cells | |
| | | | | CCR5: T cells, | |
| | | | | macrophages, dendritic | |
| | | | | cells, eosinophils and | |
| | | | | microglia | |
| | | | | D6: lymphocytes, | |
| | | | | lymphatic endothelial | |
| | | | | cells, macrophages | |
| CCL3L3 | CCL3L3 | LD78 beta | Unknown | CCR1: lymphocytes, | migration of |
| | | | | monocytes, airway | lymphocytes |
| | | | | smooth muscle cells | and monocytes |
| | | | | CCR3: eosinophils, | |
| | | | | basophils, Th2 cells, | |
| | | | | CD34+ hematopoetic | |
| | | | | progenitors, | |
| | | | | keratinocytes, mast cells | |
| | | | | CCR5: T cells, | |
| | | | | macrophages, dendritic | |
| | | | | cells, eosinophils and | |
| | | | | microglia | |
| CCL4 | CCL4 | MIP-1 beta, | macrophages, | CCR1: lymphocytes, | migration and |
| | | AT744.1, | dendritic cells | monocytes, airway | adhesion of |
| | | ACT-2, G-26, | | smooth muscle cells | lymphocytes, |
| | | HC21, H400, | | CCR5: T cells, | regulatory T |
| | | MAD-5, LAG-1 | | macrophages, dendritic | cells, NK cells, |
| | | | | cells, eosinophils and | monocyrtes |
| | | | | microglia | |
| | | | | CCR8: natural killer | |
| | | | | cells, monocytes and | |
| | | | | lymphocytes | |
| | | | | D6: lymphocytes, | |
| | | | | lymphatic endothelial | |
| | | | | cells, macrophages | |
| CCL4L1 | CCL4L1 | AT744.2 | macrophages, | CCR1: lymphocytes, | CCR1 and |
| | | | dendritic cells | monocytes, airway | CCR5 |
| | | | | smooth muscle cells | expressing |
| | | | | CCR5: T cells, | cells |
| | | | | macrophages, dendritic | |
| | | | | cells, eosinophils and | |
| | | | | microglia | |
| CCL4L2 | CCL4L2 | | macrophages, | CCR1: lymphocytes, | CCR1 and |
| | | | dendritic cells | monocytes, airway | CCR5 |
| | | | | smooth muscle cells | expressing |
| | | | | CCR5: T cells, | cells |
| | | | | macrophages, dendritic | |
| | | | | cells, eosinophils and | |
| | | | | microglia | |
| CCL5 | CCL5 | RANTES | T cells, | CCR1: lymphocytes, | migration of |
| | | | macrophages, | monocytes, airway | monocytes, |
| | | | platelets, synovial | smooth muscle cells | memory T |
| | | | fibroblasts, tubular | CCR3: eosinophils, | helper cells and |
| | | | epithelium, certain | basophils, Th2 cells, | eosinophils, |
| | | | types of tumor cells | CD34+ hematopoetic | causes the |
| | | | | progenitors, | release of |
| | | | | keratinocytes, mast cells | histamine from |
| | | | | CCR4: lymphocytes | basophils and |
| | | | | CCR5: T cells, | activates |
| | | | | macrophages, dendritic | eosinophils |
| | | | | cells, eosinophils and | |
| | | | | microglia | |
| | | | | D6: lymphocytes, | |
| | | | | lymphatic endothelial | |
| | | | | cells, macrophages | |
| | | | | DARC: erytrocytes, | |
| | | | | endothelial and epithelial | |
| | | | | cells | |
| CCL7 | CCL7 | MCP-3 | macrophages, | CCR1: lymphocytes, | migration of |
| | | | certain types of | monocytes, airway | monocytes, |
| | | | tumor cells | smooth muscle cells | activation of |
| | | | | CCR2: monocytes | macrophages |
| | | | | CCR3: eosinophils, | |
| | | | | basophils, Th2 cells, | |
| | | | | CD34+ hematopoetic | |
| | | | | progenitors, | |
| | | | | keratinocytes, mast cells | |
| | | | | D6: lymphocytes, | |
| | | | | lymphatic endothelial | |
| | | | | cells, macrophages | |
| | | | | DARC: erytrocytes, | |
| | | | | endothelial and epithelial | |
| | | | | cells | |
| CCL8 | CCL8 | MCP-2, HC14 | fibroblasts, | CCR1: lymphocytes, | migration of |
| | | | endothelial cells | monocytes, airway | monocytes, |
| | | | | smooth muscle cells | lymphocytes, |
| | | | | CCR2: monocytes | basophils and |
| | | | | CCR3: eosinophils, | eosinophils |
| | | | | basophils, Th2 cells, | |
| | | | | CD34+ hematopoetic | |
| | | | | progenitors, | |
| | | | | keratinocytes, mast cells | |
| | | | | CCR5: T cells, | |
| | | | | macrophages, dendritic | |
| | | | | cells, eosinophils and | |
| | | | | microglia | |
| | | | | CCR11: unkown | |
| | | | | D6: lymphocytes, | |
| | | | | lymphatic endothelial | |
| | | | | cells, macrophages | |
| | | | | DARC: erytrocytes, | |
| | | | | endothelial and epithelial | |
| | | | | cells | |
| CCL11 | CCL11 | Eotaxin | lung epithelial cells, | CCR3: eosinophils, | migration and |
| | | | pleural mesothelial | basophils, Th2 cells, | activation of |
| | | | cells, bronchial | CD34+ hematopoetic | inflammatory |
| | | | airway epithelial | progenitors, | leukocytes, |
| | | | cells, smooth | keratinocytes, mast cells | particularly |
| | | | muscle cells | CCR5: T cells, | eosinophils |
| | | | | macrophages, dendritic | |
| | | | | cells, eosinophils and | |
| | | | | microglia | |
| | | | | D6: lymphocytes, | |
| | | | | lymphatic endothelial | |
| | | | | cells, macrophages | |
| | | | | DARC: erytrocytes, | |
| | | | | endothelial and epithelial | |
| | | | | cells | |
| CCL12 | | | stromal cells in lung | CCR2: monocytes | migration and |
| | | | and secondary | | activation of |
| | | | lymphoid organs | | monocytes |
| CCL13 | CCL13 | MCP-4, CK | synovial fibroblasts, | CCR1: lymphocytes, | migration of |
| | | beta 10, | chondrocytes | monocytes, airway | eosinophils, |
| | | NCC-1 | | smooth muscle cells | monocytes and |
| | | | | CCR2: monocytes | T lymphocytes |
| | | | | CCR3: eosinophils, | |
| | | | | basophils, Th2 cells, | |
| | | | | CD34+ hematopoetic | |
| | | | | progenitors, | |
| | | | | keratinocytes, mast cells | |
| | | | | CCR5: T cells, | |
| | | | | macrophages, dendritic | |
| | | | | cells, eosinophils and | |
| | | | | microglia | |
| | | | | CCR11: unkown | |
| | | | | D6: lymphocytes, | |
| | | | | lymphatic endothelial | |
| | | | | cells, macrophages | |
| | | | | DARC: erytrocytes, | |
| | | | | endothelial and epithelial | |
| | | | | cells | |
| CCL14 | CCL14 | HCC-1, | spleen, bone | CCR1: lymphocytes, | activation of |
| | | MCIF, CK | marrow, liver, | monocytes, airway | monocytes |
| | | beta 1, NCC-2 | muscle and gut | smooth muscle cells | |
| | | | | CCR3: eosinophils, | |
| | | | | basophils, Th2 cells, | |
| | | | | CD34+ hematopoetic | |
| | | | | progenitors, | |
| | | | | keratinocytes, mast cells | |
| | | | | CCR5: T cells, | |
| | | | | macrophages, dendritic | |
| | | | | cells, eosinophils and | |
| | | | | microglia | |
| | | | | D6: lymphocytes, | |
| | | | | lymphatic endothelial | |
| | | | | cells, macrophages | |
| | | | | DARC: erytrocytes, | |
| | | | | endothelial and epithelial | |
| | | | | cells | |
| CCL15 | CCL15 | MIP-1 delta, | airway smooth | CCR1: lymphocytes, | migration of |
| | | LKN-1, HCC-2, | muscle cells, lung | monocytes, airway | monocytes and |
| | | MIP-5, NCC-3 | leukocytes, alveolar | smooth muscle cells | eosinophils, |
| | | | macrophages, | CCR3: eosinophils, | proliferation of |
| | | | basophils | basophils, Th2 cells, | CD34 myeloid |
| | | | | CD34+ hematopoetic | progenitor cells |
| | | | | progenitors, | |
| | | | | keratinocytes, mast cells | |
| CCL16 | CCL16 | HCC-4, LEC, | liver, thymus, and | CCR1: lymphocytes, | migration of |
| | | ILINCK, | spleen | monocytes, airway | lymphocytes |
| | | NCC-4, LMC, | | smooth muscle cells | and monocytes |
| | | CK beta 12 | | CCR2: monocytes | |
| | | | | CCR5: T cells, | |
| | | | | macrophages, dendritic | |
| | | | | cells, eosinophils and | |
| | | | | microglia | |
| | | | | CCR8: natural killer | |
| | | | | cells, monocytes and | |
| | | | | lymphocytes | |
| | | | | DARC: erytrocytes, | |
| | | | | endothelial and epithelial | |
| | | | | cells | |
| | | | | H4: bone marrow, | |
| | | | | eosinophils, T-cells, | |
| | | | | dendritic cells, | |
| | | | | monocytes, mast cells, | |
| | | | | neutrophil | |
| CCL17 | CCL17 | TARC, | constitutively | CCR4: lymphocytes | Migration and |
| | | ABCD-2 | expressed in | CCR8: natural killer | activation of T |
| | | | thymus, dendritic | cells, monocytes and | cells |
| | | | cells, keratinocytes | lymphocytes | |
| | | | | D6: lymphocytes, | |
| | | | | lymphatic endothelial | |
| | | | | cells, macrophages | |
| | | | | DARC: erytrocytes, | |
| | | | | endothelial and epithelial | |
| | | | | cells | |
| CCL18 | CCL18 | PARC, DC-CK1, | dendritic cells, | CCR8: natural killer | migration of |
| | | AMAC-1, | monocytes, and | cells, monocytes and | naive and |
| | | CK beta 7, | macrophages | lymphocytes | regulatory |
| | | MIP-4 | | PITPNM3: breast cancer | lymphocytes, |
| | | | | cells | dendritic cells |
| | | | | DARC: erytrocytes, | |
| | | | | endothelial and epithelial | |
| | | | | cells | |
| CCL19 | CCL19 | MIP-3 beta, | fibroblastic reticular | CCR7: lymphocytes | migration of |
| | | ELC, Exodus-3, | cells, dendritic cells | (mainly naive and | naive and |
| | | CK beta 11 | | memory), mature | memory |
| | | | | dendritic cells | lymphocytes |
| | | | | CCR11: unkown | and mature |
| | | | | CCRL2: neutrophils, | dendritic cells |
| | | | | monocytes | |
| CCL20 | CCL20 | MIP-3 alpha, | epidermis | CCR6: immature | migration of |
| | | LARC, | (keratinocytes), | dendritic cells and | lymphocytes, |
| | | Exodus-1, | lymphocytes | memory T cells | DCs and |
| | | ST38, CK | | | neutrophils |
| | | beta 4 | | | |
| CCL21 | CCL21 | 6Ckine, | Stromal cells, | CCR7: lymphocytes | migration of |
| | | Exodus-2, | lymphatic | (mainly naive and | lymphocytes |
| | | SLC, TCA-4, | endothelial cells, | memory), mature | homing to |
| | | CK beta 9 | fibroblastic reticular | dendritic cells | secondary |
| | | | cells, dendritic cells | CCR11: unkown | lymphoid |
| | | | | | organs, |
| | | | | | induces |
| | | | | | integrin- |
| | | | | | mediated |
| | | | | | lymphocyte |
| | | | | | adhesion |
| CCL22 | CCL22 | MDC | Macrophages | CCR4: lymphocytes | migration of NK |
| | | | | D6: lymphocytes, | cells, |
| | | | | lymphatic endothelial | chronically |
| | | | | cells, macrophages | activated T |
| | | | | | cells, |
| | | | | | monocytes and |
| | | | | | DCs |
| CCL23 | CCL23 | MPIF-1, CK | Monocytes | CCR1: lymphocytes, | migration of |
| | | beta 8, CK | | monocytes | monocytes, |
| | | beta 8-1, | | FPRL-1: monocytes, | resting T cells |
| | | MIP-3 | | mast cells | and neutrophils |
| CCL24 | CCL24 | Eotaxin-2, | lung tissue | CCR3: eosinophils, | migration of |
| | | MPIF-2, CK | | basophils, Th2 cells, | basophils |
| | | beta 6 | | CD34+ hematopoetic | |
| | | | | progenitors, | |
| | | | | keratinocytes, mast cells | |
| CCL25 | CCL25 | TECK, CK | thymic dendritic cells | CCR9: T lymphocytes of | migration of |
| | | beta 15 | and mucosal | small intestine | dendritic cells, |
| | | | epithelial cells | | thymocytes and |
| | | | | | activated |
| | | | | | macrophages |
| CCL26 | CCL26 | Eotaxin-3, | heart, lung and | CCR3: eosinophils, | migration of |
| | | MIP-4 alpha, | ovary and in | basophils, Th2 cells, | eosinophils and |
| | | IMAC, TSC-1 | endothelial cells | CD34+ hematopoetic | basophils |
| | | | stimulated with IL4 | progenitors, | |
| | | | | keratinocytes, mast cells | |
| | | | | CX3CR1: lymphocytes, | |
| | | | | monocytes | |
| CCL27 | CCL27 | CTACK, ILC, | Keratinocytes | CCR10: melanocytes, | migration of |
| | | PESKY, | | plasma cells and | memory T cells |
| | | ESKINE | | skin- homing T cells | |
| CCL28 | CCL28 | MEC | columnar epithelial | CCR3: eosinophils, | migration of |
| | | | cells in the gut, lung, | basophils, Th2 T cells, | lymphocytes |
| | | | breast and the | CD34+ hematopoetic | and eosinophils |
| | | | salivary glands | progenitors, | |
| | | | | keratinocytes, mast cells | |
| | | | | CCR10: melanocytes, | |
| | | | | plasma cells and | |
| | | | | skin- homing T cells | |

CXC Family

| | | | | | |
|---|---|---|---|---|---|
| CXCL1 | CXCL1 | GRO alpha, | mammary, | CXCR2 (IL8RB): | migration of |
| | | MGSA, | fibroblasts, | neutrophils | neutrophils |
| | | GRO1, NAP-3 | mammary epithelial | DARC: erytrocytes, | |
| | | | cells, endothelial | endothelial and epithelial | |
| | | | cells, activated, | cells | |
| | | | monocytes, | | |
| | | | macrophages and | | |
| | | | neutrophils | | |
| CXCL2 | CXCL2 | GRO beta, | monocytes, | CXCR2 (IL8RB): | migration and |
| | | MIP-2 alpha, | macrophages | neutrophils | activation of |
| | | GRO2 | | DARC: erytrocytes, | neutrophils, |
| | | | | endothelial and epithelial | basophils, |
| | | | | cells | hematopoietic |
| | | | | | stem cells |
| CXCL3 | CXCL3 | GRO gamma, | smooth muscle | CXCR2 (IL8RB): | migration and |
| | | MIP-2 beta, | cells, epithelial cells | neutrophils | activation of |
| | | GRO3 | | DARC: erytrocytes, | neutrophils |
| | | | | endothelial and epithelial | |
| | | | | cells | |
| CXCL4 | PF4 | PF4 | activated platelets, | CXCR3 (CD183b): T | migration of |
| | | | megakaryocytes, | cells, NK cells | neutrophils and |
| | | | leukocytes, | CXCR3-B: T cells, NK | fibroblasts, |
| | | | endothelial cells | cells | inhibiting |
| | | | | DARC: erytrocytes, | endothelial cell |
| | | | | endothelial and epithelial | proliferation |
| | | | | cells | and chemotaxis |
| CXCL4L1 | PF4V1 | PF4V1 | smooth muscle | CXCR3 (CD183b): T | inhibiting |
| | | | cells, T cells, and | cells, NK cells | endothelial cell |
| | | | platelets | CXCR3-B: T cells, NK | proliferation |
| | | | | cells | and chemotaxis |
| CXCL5 | CXCL5 | ENA-78 | fibroblasts, epithelial | CXCR2 (IL8RB): | migration and |
| | | | cells, eosinophils | neutrophils | activation of |
| | | | | DARC: erytrocytes, | neutrophils |
| | | | | endothelial and epithelial | |
| | | | | cells | |
| CXCL6 | CXCL6 | GCP-2 | fibroblasts, epithelial | CXCR1 (IL8RA): | migration of |
| | | | cells | neutrophils | neutrophils |
| | | | | CXCR2 (IL8RB): | |
| | | | | neutrophils | |
| | | | | DARC: erytrocytes, | |
| | | | | endothelial and epithelial | |
| | | | | cells | |
| CXCL7 | PPBP | NAP-2, | activated platelets | CXCR1 (IL8RA): | migration of |
| | | CTAPIII, | | neutrophils | neutrophils |
| | | beta-TG | | CXCR2 (IL8RB): | |
| | | | | neutrophils | |
| CXCL8 | IL8 | IL-8, NAP-1, | macrophages, | CXCR1 (IL8RA): | migration of |
| | | MDNCF, | epithelial cells, | neutrophils | neutrophils, |
| | | GCP-1 | airway smooth | CXCR2 (IL8RB): | basophils, and |
| | | | muscle cells, | neutrophils | T-cells, and |
| | | | endothelial cells | DARC: erytrocytes, | angiogenic |
| | | | | endothelial and epithelial | factor |
| | | | | cells | |
| CXCL9 | CXCL9 | MIG, CRG-10 | monocytes, | CXCR3 (CD183b): T | migration of |
| | | | macrophages and | cells, NK cells | Th1 |
| | | | endothelial cells | CXCR3-B: T cells, NK | lymphocytes, |
| | | | | cells | angiogenic |
| | | | | DARC: erytrocytes, | factor |
| | | | | endothelial and epithelial | |
| | | | | cells | |
| CXCL10 | CXCL10 | IP-10 | neutrophils, | CXCR3 (CD183b): T | migration of |
| | | | hepatocytes, | cells, NK cells | CD4+ T cells |
| | | | endothelial cells and | CXCR3-B: T cells, NK | |
| | | | keratinocytes | cells | |
| | | | | DARC: erytrocytes, | |
| | | | | endothelial and epithelial | |
| | | | | cells | |
| CXCL11 | CXCL11 | I-TAC, beta- | peripheral blood | CXCR3 (CD183b): T | migration of |
| | | R1, H174, IP-9 | leukocytes, | cells, NK cells | interleukin- activated |
| | | | pancreas and liver | CXCR7 (ACKR3): tumor | T cells but not |
| | | | astrocytes and at | cells and tumor- associated | unstimulated T |
| | | | moderate levels in | blood endothelium | cells, |
| | | | thymus, spleen and | DARC: erytrocytes, | neutrophils or |
| | | | lung | endothelial and epithelial | monocytes. |
| | | | | cells | |
| CXCL12 | CXCL12 | SDF-1, PBSF | ubiquitously | CXCR4: brain, heart, | migration of |
| | | | expressed in many | lymphocytes, HSCs, | lymphocytes |
| | | | tissues and cell | blood endothelial cells | and |
| | | | types | and umbilical cord | hepatopoietic |
| | | | | endothelial cell | stem cells, |
| | | | | CXCR7 (ACKR3): tumor | angiogenic |
| | | | | cells and tumor- associated | factor |
| | | | | blood endothelium | |
| CXCL13 | CXCL13 | BCA-1, BLC | follicles of the | CXCR3 (CD183b): T | migration of B |
| | | | spleen, lymph | cells, NK cells | cells |
| | | | nodes, and Peyer's | CXCR5: Burkitt's | |
| | | | patches | lymphoma, lymph node | |
| | | | | follicules, spleen | |
| | | | | DARC: erytrocytes, | |
| | | | | endothelial and epithelial | |
| | | | | cells | |
| CXCL14 | CXCL14 | BRAK, BMAC | Fibroblasts | unknown | migration of |
| | | | | | monocytes, NK |
| | | | | | cells, DCs |
| CXCL16 | CXCL16 | SR-PSOX | DCs | CXCR6: T cells | migration of |
| | | | | | several subsets |
| | | | | | of T cells and |
| | | | | | NKT cells |
| CXCL17 | CXCL17 | DMC, VCC-1 | Lung and tumor | unknown | migration of |
| | | | tissue | | DCs and |
| | | | | | monocytes |

TABLE 4
EXAMPLES OF HUMAN IMMUNE CELL TRAFFICKING MOLECULES

| | Trafficking | | |
|---|---|---|---|
| | molecule | | |
| Trafficking | expressing or | | Function in the extravasation |
| molecule | presenting cells | Leukocyte ligand | cascade |
| P-selectin | Blood endothelial cell | PSGL-1, L-selectin, | Tethering/Rolling during |
| | | CD44 | extravasation cascade |
| E-selectin | Blood endothelial cell | Glycoprotein, | Tethering/Rolling during |
| | | glycolipid, PSGL-1 | extravasation cascade |
| PNAd | Blood endothelial cell | L-selectin | Tethering/Rolling during |
| | | | extravasation cascade |
| MAdCAM | Blood endothelial cell | L-selectin, integrins | Tethering/Rolling, arrest during |
| | | | extravasation cascade |
| VCAM-1 | Blood endothelial cell | Integrins | Tethering/Rolling, arrest during |
| | | (e.g. VLA-4) | extravasation cascade |
| Chemokines | Blood endothelial cell | GPCRs | Integrin activation, allowing binding of |
| | | | cell adhesion molecules and arrest |
| ICAM-1 | Blood endothelial cell | Integrins | Arrest during extravasation cascade |
| | | (e.g. LFA-1, Mac-1) | |
| ICAM-2 | Blood endothelial cell | Integrins | Arrest during extravasation cascade |
| | | (e.g. LFA-1, Mac-1) | |
| PECAM1 | Blood endothelial cell | Integrins | Transmigration |
| (CD31) | | (e.g. alpha v beta 3), | |
| | | PECAM1 | |
| JAM-A/-B/-C | Blood endothelial cell | Integrins | Transmigration |
| | | (e.g. LFA-1, Mac-1, | |
| | | VLA-4) | |
| ESAM | Blood endothelial cell | unknown | Transmigration |
| CD99 | Blood endothelial cell | CD99 | Transmigration |
| CD99L2 | Blood endothelial cell | possibly CD99L | Transmigration |
| VE-cadherin | Blood endothelial cell | None | Transmigration |
| PVR | Blood endothelial cell | DNAM1 | Transmigration |
| S1P | Lymphatic endothelial | S1P receptor 1 | Entry into afferent and efferent |
| | cell | (S1P1) | lymphatics (in peripheral or SLOs |
| | | | respectively) |

### Inflammatory and Autoimmune Conditions

The methods described herein can be used to treat an inflammatory or autoimmune condition or disease in a subject in need thereof by administering an effective amount of an α6*nAChR activator to the subject. The methods described herein can further include a step of identifying (e.g., diagnosing) a subject who has an inflammatory or autoimmune condition, e.g., an inflammatory or autoimmune condition described herein. The method can include administering locally to the subject an α6*nAChR activator described herein in a dose (e.g., effective amount) and for a time sufficient to treat the autoimmune or inflammatory condition or disease.

The methods described herein can be used to inhibit an immune response in a subject in need thereof, e.g., the subject has an autoimmune condition and is in need of inhibiting an immune response against self- or auto-antibodies (e.g., the subject has Graves' disease, systemic lupus erythematosus (SLE or lupus), type 1 diabetes, multiple sclerosis (MS), plaque psoriasis, rheumatoid arthritis (RA) or another autoimmune condition described herein). The methods described herein can also include a step of selecting a subject in need of inhibiting an immune response, e.g., selecting a subject who has or who has been identified to have an inflammatory or autoimmune condition.

Types of Inflammatory and Autoimmune Conditions

In the methods described herein, the condition may be selected from: acute disseminated encephalomyelitis (ADEM); acute necrotizing hemorrhagic leukoencephalitis; Addison's disease; adjuvant-induced arthritis; agammaglobulinemia; alopecia areata; amyloidosis; ankylosing spondylitis; anti-GBM/anti-TBM nephritis; antiphospholipid syndrome (APS); autoimmune angioedema; autoimmune aplastic anemia; autoimmune dysautonomia; autoimmune gastric atrophy; autoimmune hemolytic anemia; autoimmune hepatitis; autoimmune hyperlipidemia; autoimmune immunodeficiency; autoimmune inner ear disease (AIED); autoimmune myocarditis; autoimmune oophoritis; autoimmune pancreatitis; autoimmune retinopathy; autoimmune thrombocytopenic purpura (ATP); autoimmune thyroid disease; autoimmune urticarial; axonal & neuronal neuropathies; Balo disease; Behcet's disease; bullous pemphigoid; cardiomyopathy; Castleman disease; celiac disease; Chagas disease; chronic inflammatory demyelinating polyneuropathy (CIDP); chronic recurrent multifocal ostomyelitis (CRMO); Churg-Strauss syndrome; cicatricial pemphigoid/benign mucosal pemphigoid; Crohn's disease; Cogan syndrome; collagen-induced arthritis; cold agglutinin disease; congenital heart block; coxsackie myocarditis; CREST disease; essential mixed cryoglobulinemia; demyelinating neuropathies; dermatitis herpetiformis; dermatomyositis; Devic's disease (neuromyelitis optica); discoid lupus; Dressler's syndrome; endometriosis; eosinophilic esophagitis; eosinophilic fasciitis; erythema nodosum experimental allergic encephalomyelitis; experimental autoimmune encephalomyelitis; Evans syndrome; fibromyalgia; fibrosing alveolitis; giant cell arteritis (temporal arteritis); giant cell myocarditis; glomerulonephritis; Goodpasture's syndrome; granulomatosis with polyangiitis (GPA) (formerly called Wegener's granulomatosis); Graves' disease; Guillain-Barre syndrome; Hashimoto's encephalitis; Hashimoto's thyroiditis; hemolytic anemia; Henoch-Schonlein purpura; herpes gestationis; hypogammaglobulinemia; idiopathic thrombocytopenic purpura (ITP); IgA nephropathy; IgG4-related sclerosing disease; immunoregulatory lipoproteins; inclusion body myositis; interstitial cystitis; inflammatory bowel disease; juvenile arthritis; juvenile oligoarthritis; juvenile diabetes (type 1 diabetes); juvenile myositis; Kawasaki syndrome; Lambert-Eaton syndrome; leukocytoclastic vasculitis; lichen planus; lichen sclerosus; ligneous conjunctivitis; linear IgA disease (LAD); lupus (SLE); Lyme disease, chronic; Meniere's disease; microscopic polyangiitis; mixed connective tissue disease (MCTD); Mooren's ulcer; Mucha-Habermann disease; multiple sclerosis; myasthenia gravis; myositis; arcolepsy; neuromyelitis optica (Devic's); neutropenia; non-obese diabetes; ocular cicatricial pemphigoid; optic neuritis; palindromic rheumatism; PANDAS (pediatric autoimmune neuropsychiatric disorders associated with streptococcus); paraneoplastic cerebellar degeneration; paroxysmal nocturnal hemoglobinuria (PNH); Parry Romberg syndrome; Parsonnage-Turner syndrome; pars planitis (peripheral uveitis); pemphigus; pemphigus vulgaris; peripheral neuropathy; perivenous encephalomyelitis; pernicious anemia; POEMS syndrome; polyarteritis nodosa; type I, II, & III autoimmune polyglandular syndromes; polymyalgia rheumatic; polymyositis; postmyocardial infarction syndrome; postpericardiotomy syndrome; progesterone dermatitis; primary biliary cirrhosis; primary sclerosing cholangitis; psoriasis; plaque psoriasis; psoriatic arthritis; idiopathic pulmonary fibrosis; pyoderma gangrenosum; pure red cell aplasia; Raynauds phenomenon; reactive Arthritis; reflex sympathetic dystrophy; Reiter's syndrome; relapsing polychondritis; restless legs syndrome; retroperitoneal fibrosis; rheumatic fever; rheumatoid arthritis; sarcoidosis; Schmidt syndrome; scleritis; scleroderma; sclerosing cholangitis; sclerosing sialadenitis; Sjogren's syndrome; sperm & testicular autoimmunity; stiff person syndrome; subacute bacterial endocarditis (SBE); Susac's syndrome; sympathetic ophthalmia; systemic lupus erythematosus (SLE); systemic sclerosis; Takayasu's arteritis; temporal arteritis/giant cell arteritis; thrombocytopenic purpura (TTP); Tolosa-Hunt syndrome; transverse myelitis; type 1 diabetes; ulcerative colitis; undifferentiated connective tissue disease (UCTD); uveitis; vasculitis; vesiculobullous dermatosis; vitiligo; Wegener's granulomatosis (now termed granulomatosis with polyangiitis (GPA)).

In some embodiments, the inflammatory or autoimmune disease or condition is an IFNγ-associated inflammatory or autoimmune disease or condition in which anti-IFNγ therapies have been tested (e.g., anti-IFNγ antibodies) or are in clinical development, in which agents used to treat the disease or condition have been found to reduce IFNγ, in which IFNγ has been described as a disease-causing agent, or in which IFNγ has been found to be elevated. IFNγ-associated inflammatory or autoimmune diseases or conditions in which anti-IFNγ therapies have been tested (e.g., anti-IFNγ antibodies) or are in clinical development or used for treatment, in which agents used to treat the disease or condition have been found to reduce IFNγ, or in which IFNγ has been described as a disease-causing agent include agammaglobulinemia, autoimmune aplastic anemia, autoimmune gastric atrophy, cardiomyopathy, hemolytic anemia, lichen planus, leukocytoclastic vasculitis, linear IgA disease (LAD), lupus (SLE), multiple sclerosis, myasthenia gravis, mixed connective tissue disease (MCTD), myositis, polymyositis, psoriasis, plaque psoriasis, pure red cell aplasia, vesiculobullous dermatosis, vasculitis, and vitiligo. Inflammatory or autoimmune diseases or conditions that are associated with elevated levels of IFNγ include ADEM, acute necrotizing hemorrhagic leukoencephalitis, Addison's disease, alopecia areata, amyloidosis, ankylosing spondylitis, autoimmune hepatitis, autoimmune hyperlipidemia, autoimmune immunodeficiency, autoimmune inner ear disease, autoimmune oophoritis, autoimmune pancreatitis, autoimmune retinopathy, autoimmune thrombocytopenic purpura (ATP), autoimmune thyroid disease, autoimmune urticarial, axonal & neuronal neuropathies, Behcet's disease, Castleman disease, aeliac disease, Chagas disease, chronic inflammatory demyelinating polyneuropathy, cicatricial pemphigoid/benign mucosal pemphigoid, Crohn's disease, cold agglutinin disease, congenital heart block, demyelinating neuropathies, dermatomyositis, discoid lupus, endometriosis, eosinophilic esophagitis, eosinophilic fasciitis, erythema nodosum, giant cell arteritis (temporal arteritis), granulomatosis with polyangiitis (GPA), Graves' disease, Henoch-Schonlein purpura, idiopathic thrombocytopenic purpura (ITP), IgA nephropathy, IgG4-related sclerosing disease, inclusion body myositis, inflammatory bowel disease, juvenile oligoarthritis, uvenile myositis, Kawasaki syndrome, Lambert-Eaton syndrome, Lyme disease, chronic, narcolepsy, non-obese diabetes, ocular cicatricial pemphigoid, optic neuritis, paroxysmal nocturnal hemoglobinuria (PNH), peripheral neuropathy, perivenous encephalomyelitis, progesterone dermatitis, primary biliary cirrhosis, primary sclerosing cholangitis, pyoderma gangrenosum, Raynauds phenomenon, reflex sympathetic dystrophy, relapsing polychondritis, retroperitoneal fibrosis, rheumatic fever, sarcoidosis, scleritis, scleroderma, clerosing cholangitis, Sjogren's syndrome, stiff person syndrome, sperm & testicular autoimmunity, Takayasu's arteritis, thrombocytopenic purpura (TTP), ulcerative colitis, undifferentiated connective tissue disease (UCTD), and uveitis.

In some embodiments, the inflammatory or autoimmune disease or condition is an inflammatory or autoimmune disease or condition that is associated with activated T cells, in which T cells are thought to mediate the disease or condition, in which T cell-targeted therapeutics have been employed, or in which activated T cells are observed. Inflammatory or autoimmune diseases or conditions in which T cells are thought to mediate the disease or condition or in which T cell-targeted therapeutics have been employed include alopecia areata, autoimmune aplastic anemia, autoimmune myocarditis, autoimmune retinopathy, autoimmune thrombocytopenic purpura (ATP), celiac disease, collagen-induced arthritis, Dermatomyositis, Devic's disease, eosinophilic esophagitis, giant cell myocarditis, Evans syndrome, glomerulonephritis, and autoimmune inner ear disease. Inflammatory or autoimmune diseases or conditions that are associated with activated T cells include autoimmune hemolytic anemia, autoimmune hyperlipidemia, autoimmune inner ear disease, autoimmune oophoritis, autoimmune urticarial, Balo disease, Castleman disease, chronic inflammatory demyelinating polyneuropathy, Churg-Strauss syndrome, cicatricial pemphigoid/benign mucosal pemphigoid, Crohn's disease, congenital heart block, coxsackie myocarditis, CREST disease, demyelinating neuropathies, dermatitis herpetiformis, discoid lupus, Dressler's syndrome, endometriosis, eosinophilic fasciitis, erythema nodosum, experimental autoimmune encephalomyelitis, fibrosing alveolitis, Goodpasture's syndrome, granulomatosis with polyangiitis (GPA), Graves' disease, Guillain-Barre syndrome, Hashimoto's thyroiditis, emolytic anemia, Henoch-Schonlein purpura, hypogammaglobulinemia, idiopathic thrombocytopenic purpura (ITP), IgA nephropathy, IgG4-related sclerosing disease, inflammatory bowel disease, juvenile arthritis, juvenile oligoarthritis, juvenile diabetes (Type 1 diabetes), juvenile myositis, Kawasaki syndrome, leukocytoclastic vasculitis, lichen planus, lichen sclerosus, ligneous conjunctivitis, linear IgA disease (LAD), lupus (SLE), lyme disease, chronic, microscopic polyangiitis, mixed connective tissue disease (MCTD), multiple sclerosis, myositis, narcolepsy, neutropenia, non-obese diabetes, ocular cicatricial pemphigoid, optic neuritis, paraneoplastic cerebellar degeneration, paroxysmal nocturnal hemoglobinuria (PNH), pemphigus vulgaris, POEMS syndrome, polyarteritis nodosa, polymyalgia rheumatic, polymyositis, primary biliary cirrhosis, primary sclerosing cholangitis, psoriasis, psoriatic arthritis, idiopathic pulmonary fibrosis, pyoderma gangrenosum, pure red cell aplasia, reactive arthritis, reflex sympathetic dystrophy, retroperitoneal fibrosis, relapsing polychondritis, rheumatic fever, rheumatoid arthritis, sarcoidosis, Schmidt syndrome, scleroderma, sclerosing cholangitis, sclerosing sialadenitis, Sjogren's syndrome, sperm & testicular autoimmunity, Stiff person syndrome, sympathetic ophthalmia, systemic sclerosis, Takayasu's arteritis, thrombocytopenic purpura (TTP), transverse myelitis, ulcerative colitis, uveitis, vasculitis, vesiculobullous dermatosis, and vitiligo.

Activators of nAChRs containing a nAChRα6 subunit described herein can be administered in combination with a second therapeutic agent for treatment of an inflammatory or autoimmune disease or condition. Additional therapeutic agents include, 6-mercaptopurine, 6-thioguanine, abatacept, adalimumab, alemtuzumab (Lemtrada), aminosalicylates (5-aminoalicylic acid, sulfasalazine, mesalamine, balsalazide, olsalazine), antibiotics, anti-histamines, Anti-TNFα (infliximab, adalimumab, certolizumab pegol, natalizumab), azathioprine, belimumab, beta interferon, calcineurin inhibitors, certolizumab, corticosteroids (prednisone, methylprednisolone), cromolyn, cyclosporin A, cyclosporine, dimethyl fumarate (tecfidera), etanercept, fingolimod (Gilenya), fumaric acid esters, glatiramer acetate (Copaxone), golimumab, hydroxyurea, IFNγ, IL-11, infliximab, leflunomide, leukotriene receptor antagonist, long-acting beta2 agonist, methotrexate, mitoxantrone, mycophenolate mofetil, natalizumab (tysabri), NSAIDs, ocrelizumab, pimecrolimus, probiotics (VSL#3), retinoids, rituximab, salicylic acid, short-acting beta2 agonist, sulfasalazine, tacrolimus, teriflunomide (Aubagio), theophylline, tocilizumab, ustekinumab (anti-IL12/1L23), and vedolizumab (Anti alpha3 beta7 integrin).

### Neuromodulatory Combination Therapies

Neurotransmission Modulators

In some embodiments, the α6*nAChR activator is administered in combination with a neurotransmission modulator (e.g., an agent that increases or decreases neurotransmission). A neurotransmission modulator can be used to modulate neural activity in a lymph node, secondary or tertiary lymphoid organ, or site of inflammation that is innervated by nerves or to modulate immune cells that express neurotransmitter receptors. For example, in some embodiments, the neurotransmission modulator is a neurotransmitter or neurotransmitter receptor listed in Table 5 or 6, or an agonist or antagonist listed in Tables 7A-7J for a corresponding neurotransmitter pathway member. In some embodiments, the neurotransmission modulator is a neurotransmission modulator listed in Table 8. Neurotransmission modulators that increase neurotransmission include neurotransmitters and neurotransmitter receptors listed in Tables 5 and 6 and analogs thereof, and neurotransmitter agonists (e.g., small molecules that agonize a neurotransmitter receptor listed in Table 5). Exemplary agonists are listed in Tables 7A-7J. In some embodiments, neurotransmission is increased via administration, local delivery, or stabilization of neurotransmitters (e.g., ligands listed in Tables 5 or 6). Neurotransmission modulators that increase neurotransmission also include agents that increase neurotransmitter synthesis or release (e.g., agents that increase the activity of a biosynthetic protein encoded by a gene in Table 5 via stabilization, overexpression, or upregulation, or agents that increase the activity of a synaptic or vesicular protein via stabilization, overexpression, or upregulation), prevent neurotransmitter reuptake or degradation (e.g., agents that block or antagonize transporters that remove neurotransmitter from the synaptic cleft), increase neurotransmitter receptor activity (e.g., agents that increase the activity of a signaling protein encoded by a gene in Table 5 via stabilization, overexpression, agonism, or upregulation, or agents that upregulate, agonize, or stabilize a neurotransmitter receptor listed in Table 5), increase neurotransmitter receptor synthesis or membrane insertion, decrease neurotransmitter degradation, and regulate neurotransmitter receptor conformation (e.g., agents that bind to a receptor and keep it in an “open” or “primed” conformation). In some embodiments, the neurotransmitter receptor is a channel, the activity of which can be increased by agonizing, opening, stabilizing, or overexpressing the channel. Neurotransmission modulators can increase neurotransmission by 10%, 20%, 30%, 40%, 50%, 60%, 70%, 80%, 90%, 95%, 98% or more. Exemplary neurotransmission modulators are listed in Table 8.

Neurotransmission modulators that decrease neurotransmission include neurotransmitter antagonists (e.g., small molecules that antagonize a neurotransmitter receptor listed in Table 5). Exemplary antagonists are listed in Tables 7A-7J. Neurotransmission modulators that decrease neurotransmission also include agents that decrease neurotransmitter synthesis or release (e.g., agents that decrease the activity of a biosynthetic protein encoded by a gene in Table 5 via inhibition or downregulation, or agents that decrease the activity of a synaptic or vesicular protein via blocking, disrupting, downregulating, or antagonizing the protein), increase neurotransmitter reuptake or degradation (e.g., agents that agonize, open, or stabilize transporters that remove neurotransmitter from the synaptic cleft), decrease neurotransmitter receptor activity (e.g., agents that decrease the activity of a signaling protein encoded by a gene in Table 5 or via blocking or antagonizing the protein, or agents that block, antagonize, or downregulate a neurotransmitter receptor listed in Table 5), decrease neurotransmitter receptor synthesis or membrane insertion, increase neurotransmitter degradation, regulate neurotransmitter receptor conformation (e.g., agents that bind to a receptor and keep it in a “closed” or “inactive” conformation), and disrupt the pre- or postsynaptic machinery (e.g., agents that block or disrupt a structural protein, or agents that block, disrupt, downregulate, or antagonize a synaptic or vesicular protein). In some embodiments, the neurotransmitter receptor is a channel (e.g., a ligand or voltage gated ion channel), the activity of which can be decreased by blockade, antagonism, or inverse agonism of the channel. Neurotransmission modulators that decrease neurotransmission further include agents that sequester, block, antagonize, or degrade a neurotransmitter listed in Tables 5 or 6. Neurotransmission modulators that decrease or block neurotransmission include antibodies that bind to or block the function of neurotransmitters, neurotransmitter receptor antagonists, and toxins that disrupt synaptic release. Neurotransmission modulators can decrease neurotransmission by 10%, 20%, 30%, 40%, 50%, 60%, 70%, 80%, 90%, 95%, 98% or more. Neurotransmission modulator can be administered in any of the modalities described herein (e.g., antibody, small molecule, nucleic acid, polypeptide, or viral vector).

TABLE 5
NEUROTRANSMITTER GENES & PATHWAYS

| | | | Accession | Entrez |
|---|---|---|---|---|
| Gene | Pathway | Type | Number | Gene ID |
| ABAT | Neurotransmitter | Biosynthesis | P80404 | 18 |
| ACHE | Neurotransmitter | Biosynthesis | P22303 | 43 |
| ADORA2A | Neurotransmitter | Receptor | P29274 | 135 |
| ADORA2B | Neurotransmitter | Receptor | P29275 | 136 |
| Adra1a | Adrenergic/ | Receptor | P35348 | 148 |
| | Neurotransmitter | | | |
| Adra1b | Adrenergic/ | Receptor | P35368 | 147 |
| | Neurotransmitter | | | |
| Adra1d | Adrenergic/ | Receptor | P25100 | 146 |
| | Neurotransmitter | | | |
| Adra2a | Adrenergic/ | Receptor | P08913 | 150 |
| | Neurotransmitter | | | |
| Adra2b | Adrenergic/ | Receptor | P18089 | 151 |
| | Neurotransmitter | | | |
| Adra2c | Adrenergic/ | Receptor | P18825 | 152 |
| | Neurotransmitter | | | |
| Adrb1 | Adrenergic/ | Receptor | P08588 | 153 |
| | Neurotransmitter | | | |
| Adrb2 | Adrenergic/ | Receptor | P07550 | 154 |
| | Neurotransmitter | | | |
| Adrb3 | Adrenergic/ | Receptor | P13945 | 155 |
| | Neurotransmitter | | | |
| Adrbk1 | Adrenergic | Kinase | P25098 | 156 |
| Adrbk2 | Adrenergic | Kinase | P35626 | 157 |
| BACE1 | Neurotransmitter | Biosynthesis | P56817 | 23621 |
| BCHE | Neurotransmitter | Biosynthesis | P06276 | 590 |
| BRS3 | Neuromodulator | Receptor | P32247 | P32247 |
| C6orf89 | Neuromodulator | Receptor | Q6UWU4 | 221477 |
| CHAT | Neurotransmitter | Biosynthesis | P28329 | 1103 |
| CHRFAM7A | Neurotransmitter | Receptor | Q494W8 | 89832 |
| Chrm1 | Cholinergic/ | Receptor | P11229 | 1128 |
| | Neurotransmitter | | | |
| Chrm2 | Cholinergic/ | Receptor | P08172 | 1129 |
| | Neurotransmitter | | | |
| Chrm3 | Cholinergic/ | Receptor | P20309 | 1131 |
| | Neurotransmitter | | | |
| Chrm4 | Cholinergic/ | Receptor | P08173 | 1132 |
| | Neurotransmitter | | | |
| Chrm5 | Cholinergic/ | Receptor | P08912 | 1133 |
| | Neurotransmitter | | | |
| Chrna1 | Cholinergic/ | Receptor | P02708 | 1134 |
| | Neurotransmitter | | | |
| Chrna10 | Cholinergic/ | Receptor | Q9GZZ6 | 57053 |
| | Neurotransmitter | | | |
| Chrna2 | Cholinergic/ | Receptor | Q15822 | 1135 |
| | Neurotransmitter | | | |
| Chrna3 | Cholinergic/ | Receptor | P32297 | 1136 |
| | Neurotransmitter | | | |
| Chrna4 | Cholinergic/ | Receptor | P43681 | 1137 |
| | Neurotransmitter | | | |
| Chrna5 | Cholinergic/ | Receptor | P30532 | 1138 |
| | Neurotransmitter | | | |
| Chrna7 | Cholinergic/ | Receptor | P36544 | 1139 |
| | Neurotransmitter | | | |
| Chrna9 | Cholinergic/ | Receptor | Q9UGM1 | 55584 |
| | Neurotransmitter | | | |
| Chrnb1 | Cholinergic/ | Receptor | P11230 | 1140 |
| | Neurotransmitter | | | |
| Chrnb2 | Cholinergic/ | Receptor | P17787 | 1141 |
| | Neurotransmitter | | | |
| Chrnb3 | Cholinergic/ | Receptor | Q05901 | 1142 |
| | Neurotransmitter | | | |
| Chrnb4 | Cholinergic/ | Receptor | P30926 | 1143 |
| | Neurotransmitter | | | |
| Chrnd | Cholinergic/ | Receptor | Q07001 | 1144 |
| | Neurotransmitter | | | |
| Chrne | Cholinergic/ | Receptor | Q04844 | 1145 |
| | Neurotransmitter | | | |
| Chrng | Cholinergic/ | Receptor | P07510 | 1146 |
| | Neurotransmitter | | | |
| CNR1 | Cannabinoid/ | Receptor | P21554 | 1268 |
| | Neurotransmitter | | | |
| CNR2 | Cannabinoid/ | Receptor | P34972 | 1269 |
| | Neurotransmitter | | | |
| CNRIP1 | Neurotransmitter | Receptor | Q96F85 | 25927 |
| COMT | Neurotransmitter | Biosynthesis | P21964 | 1312 |
| CPA4 | Neurotransmitter | Biosynthesis | Q9UI42 | 51200 |
| CPE | Neuropeptide/ | Biosynthesis | P16870 | 1363 |
| | Neurotransmitter | | | |
| CREM | Neurotransmitter | Signaling | Q03060 | 1390 |
| DAGLA | Neurotransmitter | Biosynthesis | Q9Y4D2 | 747 |
| | (Cannabinoid) | | | |
| DAGLB | Neurotransmitter | Biosynthesis | Q8NCG7 | 221955 |
| | (Cannabinoid) | | | |
| DBH | Neurotransmitter | Biosynthesis | P09172 | 1621 |
| DDC | Neurotransmitter | Biosynthesis | P20711 | 1644 |
| DGKI | Neurotransmitter | Biosynthesis | O75912 | 9162 |
| DOPO | Dopaminergic | Receptor | P09172 | 1621 |
| DPP4 | Neurotransmitter | Biosynthesis | P27487 | 1803 |
| Drd1 | Dopaminergic/ | Receptor | P21728 | 1812 |
| | Neurotransmitter | | | |
| Drd2 | Dopaminergic/ | Receptor | P14416 | 1813 |
| | Neurotransmitter | | | |
| Drd3 | Dopaminergic/ | Receptor | P35462 | 1814 |
| | Neurotransmitter | | | |
| Drd4 | Dopaminergic/ | Receptor | P21917 | 1815 |
| | Neurotransmitter | | | |
| Drd5 | Dopaminergic/ | Receptor | P21918 | 1816 |
| | Neurotransmitter | | | |
| ECEL1 | Neurotransmitter | Biosynthesis | O95672 | 9427 |
| FAAH | Neurotransmitter | Biosynthesis | O00519 | 2166 |
| FNTA | Neurotransmitter | Signaling | P49354 | 2339 |
| GABARAP | Neurotransmitter | Receptor | O95166 | 11337 |
| GABARAPL1 | Amine | Receptor | Q9H0R8 | 23710 |
| | Neuromodulator | | | |
| GABARAPL2 | Amine | Receptor | P60520 | 11345 |
| | Neuromodulator | | | |
| GABBR1 | Neurotransmitter | Receptor | Q9UBS5 | 2550 |
| GABBR2 | Amine | Receptor | O75899 | 9568 |
| | Neuromodulator | | | |
| GABRA1 | Neurotransmitter | Receptor | P14867 | 2554 |
| GABRA2 | Neurotransmitter | Receptor | P47869 | 2555 |
| GABRA3 | Neurotransmitter | Receptor | P34903 | 2556 |
| GABRA4 | Neurotransmitter | Receptor | P48169 | 2557 |
| GABRA5 | Neurotransmitter | Receptor | P31644 | 2558 |
| GABRA6 | Neurotransmitter | Receptor | Q16445 | 2559 |
| GABRB1 | Neurotransmitter | Receptor | P18505 | 2560 |
| GABRB2 | Neurotransmitter | Receptor | P47870 | 2561 |
| GABRB3 | Neurotransmitter | Receptor | P28472 | 2562 |
| GABRD | Neurotransmitter | Receptor | O14764 | 2563 |
| GABRE | Neurotransmitter | Receptor | P78334 | 2564 |
| GABRG1 | Neurotransmitter | Receptor | Q8N1C3 | 2565 |
| GABRG2 | Neurotransmitter | Receptor | P18507 | 2566 |
| GABRG3 | Neurotransmitter | Receptor | Q99928 | 2567 |
| GABRP | Neurotransmitter | Receptor | O00591 | 2568 |
| GABRQ | Neurotransmitter | Receptor | Q9UN88 | 55879 |
| GABRR1 | Neurotransmitter | Receptor | P24046 | 2569 |
| GABRR2 | Neurotransmitter | Receptor | P28476 | 2570 |
| GABRR3 | Neurotransmitter | Receptor | A8MPY1 | 200959 |
| GAD1 | Neurotransmitter | Biosynthesis | Q99259 | 2571 |
| GAD2 | Neurotransmitter | Biosynthesis | Q05329 | 2572 |
| GCHFR | Neurotransmitter | Biosynthesis | P30047 | 2644 |
| GLRA1 | Neurotransmitter | Receptor | P23415 | 2741 |
| GLRA2 | Neurotransmitter | Receptor | P23416 | 2742 |
| GLRA3 | Neurotransmitter | Receptor | O75311 | 8001 |
| GLRA4 | Neurotransmitter | Receptor | Q5JXX5 | 441509 |
| GLRB | Neurotransmitter | Receptor | P48167 | 2743 |
| GLS | Neurotransmitter | Biosynthesis | O94925 | 2744 |
| GLS2 | Neurotransmitter | Biosynthesis | Q9UI32 | 27165 |
| GluA1 (GluR1) | Amine | Receptor | P42261 | 2890 |
| | Neuromodulator | | | |
| GluK1 (GluR5) | Amine | Receptor | P39086 | 2897 |
| | Neuromodulator | | | |
| GLUL | Neurotransmitter | Biosynthesis | P15104 | 2752 |
| GluN1(NR1) | Amine | Receptor | Q05586 | 2902 |
| | Neuromodulator | | | |
| GNMT | Neurotransmitter | Biosynthesis | Q14749 | 27232 |
| GPER1 | Neurotransmitter | Receptor | Q99527 | 2852 |
| GPR1 | Neurotransmitter | Receptor | P46091 | 2825 |
| GPR139 | Neurotransmitter | Receptor | Q6DWJ6 | 124274 |
| GPR143 | Neurotransmitter | Receptor | P51810 | 4935 |
| GPR149 | Neurotransmitter | Receptor | Q86SP6 | 344758 |
| GPR18 | Neurotransmitter | Receptor | Q14330 | 2841 |
| GPR21 | Neurotransmitter | Receptor | Q99679 | 2844 |
| GPR26 | Neurotransmitter | Receptor | Q8NDV2 | 2849 |
| GPR3 | Neurotransmitter | Receptor | P46089 | 2827 |
| GPR35 | Neurotransmitter | Receptor | Q9HC97 | 2859 |
| GPR52 | Neurotransmitter | Receptor | Q9Y2T5 | 9293 |
| GPR55 | Neurotransmitter | Receptor | Q9Y2T6 | 9290 |
| GPR78 | Neurotransmitter | Receptor | Q96P69 | 27201 |
| GPR83 | Neurotransmitter | Receptor | Q9NYM4 | 10888 |
| GPR84 | Neurotransmitter | Receptor | Q9NQS5 | 53831 |
| GPRASP1 | Neurotransmitter | Receptor | Q5JY77 | 9737 |
| GPR50 | Amine | Receptor | Q13585 | 9248 |
| | Neuromodulator | | | |
| GRIA1 | Neurotransmitter | Receptor | P42261 | 2890 |
| GRIA2 | Neurotransmitter | Receptor | P42262 | 2891 |
| GRIA3 | Neurotransmitter | Receptor | P42263 | 2892 |
| GRIA4 | Neurotransmitter | Receptor | P48058 | 2893 |
| GRID1 | Neurotransmitter | Receptor | Q9ULK0 | 2894 |
| GRID2 | Neurotransmitter | Receptor | O43424 | 2895 |
| GRIK1 | Neurotransmitter | Receptor | P39086 | 2897 |
| GRIK2 | Neurotransmitter | Receptor | Q13002 | 2898 |
| GRIK3 | Neurotransmitter | Receptor | Q13003 | 2899 |
| GRIK4 | Neurotransmitter | Receptor | Q16099 | 2900 |
| GRIK5 | Neurotransmitter | Receptor | Q16478 | 2901 |
| GRIN1 | Neurotransmitter | Receptor | Q05586 | 2902 |
| GRIN2A | Neurotransmitter | Receptor | Q12879 | 2903 |
| GRIN2B | Neurotransmitter | Receptor | Q13224 | 2904 |
| GRIN2C | Neurotransmitter | Receptor | Q14957 | 2905 |
| GRIN2D | Neurotransmitter | Receptor | O15399 | 2906 |
| GRIN3A | Neurotransmitter | Receptor | Q8TCU5 | 116443 |
| GRIN3B | Neurotransmitter | Receptor | O60391 | 116444 |
| GRK2 | Neurotransmitter | Receptor | P25098 | 156 |
| GRK3 | Neurotransmitter | Receptor | P35626 | 157 |
| GRM1 | Neurotransmitter | Receptor | Q13255 | 2911 |
| GRM2 | Neurotransmitter | Receptor | Q14416 | 2912 |
| GRM3 | Neurotransmitter | Receptor | Q14832 | 2913 |
| GRM4 | Neurotransmitter | Receptor | Q14833 | 2914 |
| GRM5 | Neurotransmitter | Receptor | P41594 | 2915 |
| GRM6 | Neurotransmitter | Receptor | O15303 | 2916 |
| GRM7 | Neurotransmitter | Receptor | Q14831 | 2917 |
| GRM8 | Neurotransmitter | Receptor | O00222 | 2918 |
| HNMT | Neurotransmitter | Biosynthesis | P50135 | 3176 |
| HOMER1 | Neurotransmitter | Receptor | Q86YM7 | 9456 |
| HRH1 | Neurotransmitter | Receptor | P35367 | 3269 |
| HRH2 | Neurotransmitter | Receptor | P25021 | 3274 |
| HRH3 | Neurotransmitter | Receptor | Q9Y5N1 | 11255 |
| HRH4 | Neurotransmitter | Receptor | Q9H3N8 | 59340 |
| Htr1a | Neurotransmitter | Receptor | P08908 | 3350 |
| Htr1b | Neurotransmitter | Receptor | P28222 | 3351 |
| Htr1c | Neurotransmitter | Receptor | P28335 | |
| Htr1d | Neurotransmitter | Receptor | P28221 | 3352 |
| Htr1e | Neurotransmitter | Receptor | P28566 | 3354 |
| Htr1f | Neurotransmitter | Receptor | P30939 | 3355 |
| Htr2a | Neurotransmitter | Receptor | P28223 | 3356 |
| Htr2b | Neurotransmitter | Receptor | P41595 | 3357 |
| Htr2c | Neurotransmitter | Receptor | P28335 | 3358 |
| Htr3a | Neurotransmitter | Receptor | P46098 | 3359 |
| Htr3b | Neurotransmitter | Receptor | O95264 | 9177 |
| Htr3c | Neurotransmitter | Receptor | Q8WXA8 | 170572 |
| Htr3d | Neurotransmitter | Receptor | Q70Z44 | 200909 |
| HTR3E | Neurotransmitter | Receptor | A5X5Y0 | 285242 |
| Htr4 | Neurotransmitter | Receptor | Q13639 | 3360 |
| Htr5a | Neurotransmitter | Receptor | P47898 | 3361 |
| Htr5b | Neurotransmitter | Receptor | P35365 | 79247 |
| HTR5BP | Neurotransmitter | Receptor | | 645694 |
| Htr6 | Neurotransmitter | Receptor | P50406 | 3362 |
| Htr7 | Neurotransmitter | Receptor | P32305 | 3363 |
| ITPR1 | Neurotransmitter | Signaling | Q14643 | 3708 |
| ITPR2 | Neurotransmitter | Signaling | Q14571 | 3709 |
| ITPR3 | Neurotransmitter | Signaling | Q14573 | 3710 |
| LYNX1 | Neurotransmitter | Receptor | Q9BZG9 | 66004 |
| MAOA | Neurotransmitter | Biosynthesis | P21397 | 4128 |
| MAOB | Neurotransmitter | Biosynthesis | P27338 | 4129 |
| NAMPT | Neurotransmitter | Biosynthesis | P43490 | 10135 |
| NISCH | Neurotransmitter | Receptor | Q9Y2I1 | 11188 |
| NOS1 | Neurotransmitter | Biosynthesis | P29475 | 4842 |
| NPTN | Neurotransmitter | Receptor | Q9Y639 | 27020 |
| P2RX1 | Neurotransmitter | Receptor | P51575 | 5023 |
| P2RX2 | Neurotransmitter | Receptor | Q9UBL9 | 22953 |
| P2RX3 | Neurotransmitter | Receptor | P56373 | 5024 |
| P2RX4 | Neurotransmitter | Receptor | Q99571 | 5025 |
| P2RX5 | Neurotransmitter | Receptor | Q93086 | 5026 |
| P2RX6 | Neurotransmitter | Receptor | O15547 | 9127 |
| P2RX7 | Neurotransmitter | Receptor | Q99572 | 5027 |
| P2RY11 | Neurotransmitter | Receptor | Q96G91 | 5032 |
| PAH | Neurotransmitter | Biosynthesis | P00439 | 5053 |
| PC | Neurotransmitter | Biosynthesis | P11498 | 5091 |
| PDE1B | Neurotransmitter | Signaling | Q01064 | 5153 |
| PDE4A | Neurotransmitter | Signaling | P27815 | 5141 |
| PDE4D | Neurotransmitter | Signaling | Q08499 | 5144 |
| PHOX2A | Neurotransmitter | Biosynthesis | O14813 | 401 |
| PHOX2B | Neurotransmitter | Biosynthesis | Q99453 | 8929 |
| PIK3CA | Neurotransmitter | Signaling | P42336 | 5290 |
| PIK3CB | Neurotransmitter | Signaling | P42338 | 5291 |
| PIK3CG | Neurotransmitter | Signaling | P48736 | 5294 |
| PLCB1 | Neurotransmitter | Signaling | Q9NQ66 | 23236 |
| PLCB2 | Neurotransmitter | Signaling | Q00722 | 5330 |
| PLCB3 | Neurotransmitter | Signaling | Q01970 | 5331 |
| PLCB4 | Neurotransmitter | Signaling | Q15147 | 5332 |
| PLCD1 | Neurotransmitter | Signaling | P51178 | 5333 |
| PLCE1 | Neurotransmitter | Signaling | Q9P212 | 51196 |
| PLCG1 | Neurotransmitter | Signaling | P19174 | 5335 |
| PLCL1 | Neurotransmitter | Signaling | Q15111 | 5334 |
| PLCL2 | Neurotransmitter | Signaling | Q9UPR0 | 23228 |
| PPP1CB | Neurotransmitter | Signaling | P62140 | 5500 |
| PPP1CC | Neurotransmitter | Signaling | P36873 | 5501 |
| PRIMA1 | Neurotransmitter | Biosynthesis | Q86XR5 | 145270 |
| PRKACG | Neurotransmitter | Signaling | P22612 | 5568 |
| PRKAR2B | Neurotransmitter | Signaling | P31323 | 5577 |
| PRKCG | Neurotransmitter | Signaling | P05129 | 5582 |
| PRKX | Neurotransmitter | Signaling | P51817 | 5613 |
| RIC3 | Neurotransmitter | Receptor | Q7Z5B4 | 79608 |
| SHANK3 | Neurotransmitter | Signaling | Q9BYB0 | 85358 |
| SLC6A1 | Amine | Transferase | P30531 | 6529 |
| | Neuromodulator | | | |
| SLC6A13 | Amine | Transferase | Q9NSD5 | 6540 |
| | Neuromodulator | | | |
| Slc6a4 | Serotonin | Transporter | P31645 | 6532 |
| SNX13 | Neurotransmitter | Signaling | Q9Y5W8 | 23161 |
| TAAR1 | Amine | Receptor | Q96RJ0 | 134864 |
| | Neuromodulator | | | |
| TAAR2 | Amine | Receptor | Q9P1P5 | 9287 |
| | Neuromodulator | | | |
| TAAR5 | Neurotransmitter | Receptor | O14804 | 9038 |
| TH | Neurotransmitter | Biosynthesis | P07101 | 7054 |
| TPH1 | Neurotransmitter | Biosynthesis | P17752 | 7166 |
| TPH2 | Neurotransmitter | Biosynthesis | Q8IWU9 | 121278 |
| TRHDE | Neurotransmitter | Biosynthesis | Q9UKU6 | 29953 |

TABLE 6
NEUROTRANSMITTERS

| Ligand | Pathway | Type |
|---|---|---|
| 2-Arachidonoylglycerol | Endocannabinoid | Ligand |
| 2-Arachidonyl glyceryl ether | Endocannabinoid | Ligand |
| 3-methoxytyramine | Amines | Ligand |
| Acetylcholine | Amino Acids | Ligand |
| Adenosine | Purine | Ligand |
| Adenosine triphosphate | Purine | Ligand |
| Agmatine | Amino Acids | Ligand |
| Anandamide | Endocannabinoid | Ligand |
| Aspartate | Amino Acids | Ligand |
| Carbon monoxide | Gas | Ligand |
| D-serine | Amino Acids | Ligand |
| Dopamine | Monoamines | Ligand |
| Dynorphin | Opioids | Ligand |
| Endorphin | Opioids | Ligand |
| Enkephalin | Opioids | Ligand |
| Epinephrine | Monoamines | Ligand |
| Gamma-aminobutyric acid | Amino Acids | Ligand |
| Glutamate | Amino Acids | Ligand |
| Glycine | Amino Acids | Ligand |
| Histamine | Monoamines | Ligand |
| N-Acetylaspartylglutamate | Neuropeptides | Ligand |
| N-Arachidonoyl dopamine | Endocannabinoid | Ligand |
| N-methylphenethylamine | Amines | Ligand |
| N-methyltryptamine | Amines | Ligand |
| Nitric oxide | Gas | Ligand |
| Norepinephrine | Monoamines | Ligand |
| Octopamine | Amines | Ligand |
| Phenethylamine | Amines | Ligand |
| Serotonin | Monoamines | Ligand |
| Synephrine | Amines | Ligand |
| Tryptamine | Amines | Ligand |
| Tyramine | Amines | Ligand |
| Virodhamine | Endocannabinoid | Ligand |

TABLE 7A
AGONISTS AND ANTAGONIST AGENTS

| Gene | Agonist | Antagonist |
|---|---|---|
| Adrb2 | NCX 950 | Alprenolol |
| Accession Number: | Bitolterol | Carvedilol |
| P07550 | Isoetarine | Desipramine |
| | Norepinephrine | Nadolol |
| | Phenylpropanolamine | Levobunolol |
| | Dipivefrin | Metipranolol |
| | Epinephrine | Bevantolol |
| | Orciprenaline | Oxprenolol |
| | Dobutamine | Nebivolol |
| | Ritodrine | Asenapine |
| | Terbutaline | Bupranolol |
| | Salmeterol | Penbutolol |
| | Formoterol | Celiprolol |
| | Salbutamol | Pindolol |
| | Isoprenaline | Acebutolol |
| | Arbutamine | Bopindolol |
| | Arformoterol | |
| | Fenoterol | |
| | Pirbuterol | |
| | Ephedra | |
| | Procaterol | |
| | Clenbuterol | |
| | Bambuterol | |
| | Indacaterol | |
| | Droxidopa | |
| | Olodaterol | |
| | Vilanterol | |
| | Pseudoephedrine | |
| | Cabergoline | |
| | Mirtazepine | |
| Adra1d | Midodrine | Dapiprazole |
| Accession Number: | Norepinephrine | Amitriptyline |
| P25100 | Clonidine | Alfuzosin |
| | Oxymetazoline | Promazine |
| | Pergolide | Prazosin |
| | Bromocriptine | Imipramine |
| | Droxidopa | Nortriptyline |
| | Xylometazoline | Doxazosin |
| | Ergotamine | Nicardipine |
| | Cirazoline | Dronedarone |
| | Cabergoline | Tamsulosin |
| | Methoxamine | Propiomazine |
| | Epinephrine | Phenoxybenzamine |
| | | Carvedilol |
| | | Doxepin |
| | | Terazosin |
| | | Quetiapine |
| | | Methotrimeprazine |
| | | Silodosin |
| Adrb1 | Isoetarine | Esmolol |
| Accession Number: | Norepinephrine | Betaxolol |
| P08588 | Phenylpropanolamine | Metoprolol |
| | Epinephrine | Atenolol |
| | Dobutamine | Timolol |
| | Salbutamol | Sotalol |
| | Isoprenaline | Propranolol |
| | Arbutamine | Labetalol |
| | Fenoterol | Bisoprolol |
| | Pirbuterol | Alprenolol |
| | Ephedra | Amiodarone |
| | Clenbuterol | Carvedilol |
| | Droxidopa | Nadolol |
| | Pseudoephedrine | Levobunolol |
| | Carteolol | Metipranolol |
| | Cabergoline | Bevantolol |
| | Mirtazapine | Practolol |
| | Loxapine | Oxprenolol |
| | Vortioxetine | Celiprolol |
| | Desipramine | Nebivolol |
| | | Asenapine |
| | | Bupranolol |
| | | Penbutolol |
| | | Pindolol |
| | | Acebutolol |
| | | Bopindolol |
| | | Cartelol |
| Adrb3 | SR 58611 | Bopindolol |
| Accession Number: | Norepinephrine | Propranolol |
| P13945 | Epinephrine | Bupranolol |
| | Isoprenaline | |
| | Arbutamine | |
| | Fenoterol | |
| | Ephedra | |
| | Clenbuterol | |
| | Droxidopa | |
| | Mirabegron | |
| Adrbk1 | ATP | Alprenolol |
| Accession Number: | Carbachol | Heparin |
| P25098 | Dopamine | |
| | Isoproterenol | |
| | Morphine | |
| | DAMGO | |
| | histamine | |
| | Acetylcholine | |
| | Etorphine | |
| | NMDA | |
| | Dopamine | |
| Adrbk2 | Isoproterenol | Propranolol |
| Accession Number: | DAMGO | |
| P26819 | ATP | |
| Chrm3 | cgmp | MT3 |
| Accession Number: | ATP | Hexocyclium |
| P20309 | Cevimeline | Himbacine |
| | arecoline | Biperiden |
| | oxotremorine-M | lithocholylcholine |
| | NNC 11-1314 | AFDX384 |
| | xanomeline | 4-DAMP |
| | oxotremorine | hexahydrodifenidol |
| | pentylthio-TZTP | VU0255035 |
| | arecaidine propargyl ester | N-methyl scopolamine |
| | NNC 11-1607 | Darifenacin |
| | furmethide | Thiethylperazine |
| | NNC 11-1585 | methoctramine |
| | Acetylcholine | silahexocyclium |
| | methylfurmethide | Strychnine |
| | Bethanechol | MT7 |
| | Carbachol | Heparin |
| | Succinylcholine | Olanzapine |
| | ALKS 27 | Pirenzepine |
| | itopride | Clidinium |
| | methacholine | Ipratropium |
| | Meperidine | Propantheline |
| | Cinnarizine | Dicyclomine |
| | Trimipramine | Darifenacin |
| | | Tiotropium |
| | | Atropine |
| | | Scopolamine |
| | | Amitriptyline |
| | | Doxepin |
| | | Lidocaine |
| | | Nortriptyline |
| | | Tropicamide |
| | | Metixene |
| | | Homatropine Methylbromide |
| | | Solifenacin |
| | | Glycopyrrolate |
| | | Propiomazine |
| | | Diphemanil Methylsulfate |
| | | Promethazine |
| | | Diphenidol |
| | | Pancuronium |
| | | Ziprasidone |
| | | Quetiapine |
| | | Imipramine |
| | | Clozapine |
| | | Cyproheptadine |
| | | Aripiprazole |
| | | Nicardipine |
| | | Amoxapine |
| | | Loxapine |
| | | Promazine |
| | | Oxyphencyclimine |
| | | Anisotropine Methylbromide |
| | | Tridihexethyl |
| | | Chlorpromazine |
| | | Ketamine |
| | | Cyclosporin A |
| | | Paroxetine |
| | | Benzquinamide |
| | | Tolterodine |
| | | Oxybutynin |
| | | Alcuronium |
| | | WIN 62, 577 |
| | | Tramadol |
| | | Chlorprothixene |
| | | Aclidinium |
| | | Methotrimeprazine |
| | | Umeclidinium |
| | | Cryptenamine |
| | | Mepenzolate |
| | | Maprotiline |
| | | Brompheniramine |
| | | Isopropamide |
| | | Trihexyphenidyl |
| | | Ipratropium bromide |
| | | Hyoscyamine |
| | | Procyclidine |
| | | Pipecuronium |
| | | Fesoterodine |
| | | Disopyramide |
| | | Desipramine |
| | | Mivacurium |
| Chrna3 | Nicotine | A-867744 |
| Accession Number: | Varenicline | NS1738 |
| P32297 | Acetylcholine | Hexamethonium |
| | Ethanol | Mecamylamine |
| | Cytisine | Dextromethorphan |
| | Levamisole | Pentolinium |
| | Galantamine | Levomethadyl Acetate |
| | | Bupropion |
| Chrna9 | Nicotine | Hexamethonium |
| Accession Number: | Galantamine | Mecamylamine |
| Q9UGM1 | Ethanol | Tetraethylammonium |
| | | Muscarine |
| | ATG003 | Strychnine |
| | Lobeline | |
| | RPI-78M | |
| Chrnb1 | Galantamine | |
| Accession Number: | | |
| P11230 | | |
| Chrnb4 | Nicotine | Atropine |
| Accession Number: | Varenicline | Oxybutynin |
| P30926 | PNU-120596 | Pentolinium |
| | Ethanol | Dextromethorphan |
| | Galantamine | |
| Chrng | Galantamine | |
| Accession Number: | | |
| P07510 | | |
| Adcyap1 | Nicotine | Atropine |
| Accession Number: | CGMP | PPADS |
| P18509 | Apomorphine | Onapristone |
| | Suramin | Muscarine |
| | Nifedipine | Haloperidol |
| | ATP | Astressin |
| | Dihydrotestosterone | Melatonin |
| | Maxadilan | Scopolamine |
| | Dexamethasone | Tetrodotoxin |
| | Acetylcholine | Apamin |
| | Histamine | Hexamethonium |
| | Carbachol | Indomethacin |
| | NMDA | Propranolol |
| | Dopamine | Bumetanide |
| | Isoproterenol | Progesterone |
| | Salbutamol | Charybdotoxin |
| | Morphine | Prazosin |
| | Clonidine | |
| | Nimodipine | |
| | 2,6-Diamino-Hexanoic Acid Amide | |
| CYSLTR1 | Salbutamol | Montelukast |
| Accession Number: | Dexamethasone | Zafirlukast |
| Q9Y271 | Arachidonic acid | Cinalukast |
| | Histamine | Pranlukast |
| | | Nedocromil |
| | | Theophylline |
| | | Indomethacin |
| | | Zileuton |
| | | Iralukast |
| | | Pobilukast |
| | | Sulukast |
| | | Verlukast |
| LTB4R | LTB | U75302 |
| Accession Number: | ATP | CP105696 |
| Q15722 | Dexamethasone | CP-195543 |
| | cholesterol | Etalocib |
| | 20-hydroxy-LTB< | SC-41930 |
| | 12R-HETE | LY255283 |
| | arachidonic acid | Zafirlukast |
| | | ONO-4057 |
| | | RO5101576 |
| | | BILL 260 |
| PENK | Dopamine | Naltrexone |
| Accession Number: | kainate | Naloxone |
| P01210 | NMDA | Progesterone |
| | DAMGO | |
| | Morphine | |
| Htr2c | Apomorphine | Melatonin |
| Accession Number: | Bifeprunox | SB 224289 |
| P28335 | Tramadol | LY334362 |
| | AL-37350A | FR260010 |
| | 5-MeO-DMT | Sulpiride |
| | BW723C86 | Thiethylperazine |
| | CGS-12066 | cyamemazine |
| | DOI | Mesulergine |
| | 5-CT | SB 221284 |
| | YM348 | Zotepine |
| | LSD | Metergoline |
| | xanomeline | methiothepin |
| | WAY-163909 | Spiperone |
| | Dopamine | SB 215505 |
| | LY344864 | Tiospirone |
| | VER-3323 | SB 228357 |
| | TFMPP | Pizotifen |
| | 8-OH-DPAT | SB 206553 |
| | MK-212 | SB 204741 |
| | NMDA | SDZ SER-082 |
| | org 12962 | Ritanserin |
| | 5-MeOT | SB 242084 |
| | RU 24969 | S33084 |
| | Acetylcholine | Roxindole |
| | QUINPIROLE | RS-127445 |
| | quipazine | Terguride |
| | tryptamine | EGIS-7625 |
| | Ro 60-0175 | SB 243213 |
| | Oxymetazoline | RS-102221 |
| | Ergotamine | Olanzapine |
| | Cabergoline | Aripiprazole |
| | Lorcaserin | Agomelatine |
| | Pergolide | Ziprasidone |
| | Methylergonovine | Quetiapine |
| | Renzapride | Sarpogrelate |
| | Pramipexole | Perphenazine |
| | GR-127935 | Thioridazine |
| | BRL-15572 | Sertindole |
| | ipsapirone | Loxapine |
| | SB 216641 | Methysergide |
| | SL65.0155 | Risperidone |
| | S 16924 | Asenapine |
| | Bromocriptine | Mianserin |
| | Lisuride | Clozapine |
| | Tegaserod | Trifluoperazine |
| | Epicept NP-1 | Trazodone |
| | dapoxetine | Doxepin |
| | Dexfenfluramine | Nortriptyline |
| | 3,4- | Chlorprothixene |
| | Methylenedioxymethamphetamine | |
| | Ropinirole | Minaprine |
| | Maprotiline | Propiomazine |
| | Desipramine | Mirtazapine |
| | | Amoxapine |
| | | Yohimbine |
| | | Cyproheptadine |
| | | Imipramine |
| | | Amitriptyline |
| | | Promazine |
| | | Chlorpromazine |
| | | Ketamine |
| | | Propranolol |
| | | Fluoxetine |
| | | Ketanserin |
| | | Mesulergine |
| | | AC-90179 |
| | | Ergoloid mesylate 2 |
| | | Methotrimeprazine |
| | | Paliperidone |
| | | Clomipramine |
| | | Trimipramine |
| | | Captodiame |
| | | Nefazodone |
| GABA Receptor | Bamaluzole | bicuculline |
| Accession Numbers | GABA | Metrazol |
| (Q9UBS5, O95166, | Gabamide | Flumazenil |
| O75899, P28472, P18507, | GABOB | Thiothixine |
| P47870, P47869, O14764) | Gaboxadol | Bupropion |
| | Ibotenic acid | Caffeine |
| | Isoguvacine | |
| | Isonipecotic acid | |
| | Muscimol | |
| | Phenibut | |
| | Picamilon | |
| | Progabide | |
| | Quisqualamine | |
| | SL 75102 | |
| | Thiomuscimol | |
| | Alcohols (e.g., ethanol, isopropanol) | |
| | Avermectins (e.g., ivermectin) | |
| | Barbiturates (e.g., phenobarbital) | |
| | Benzodiazepines | |
| | Bromides (e.g., potassium bromide | |
| | Carbamates (e.g., meprobamate, | |
| | carisoprodol) | |
| | Chloralose | |
| | Chlormezanone | |
| | Clomethiazole | |
| | Dihydroergolines (e.g., ergoloid | |
| | (dihydroergotoxine)) | |
| | Etazepine | |
| | Etifoxine | |
| | Imidazoles (e.g., etomidate) | |
| | Kavalactones (found in kava) | |
| | Loreclezole | |
| | Neuroactive steroids (e.g., | |
| | allopregnanolone, ganaxolone) | |
| | Nonbenzodiazepines (e.g., | |
| | zaleplon, zolpidem, zopiclone, | |
| | eszopiclone) | |
| | Petrichloral | |
| | Phenols (e.g., propofol) | |
| | Piperidinediones (e.g., glutethimide, | |
| | methyprylon) | |
| | Propanidid | |
| | Pyrazolopyridines (e.g., etazolate) | |
| | Quinazolinones (e.g., | |
| | methaqualone) | |
| | Skullcap constituents | |
| | Stiripentol | |
| | Sulfonylalkanes (e.g., | |
| | sulfonmethane, tetronal, trional) | |
| | Valerian constituents (e.g., valeric | |
| | acid, valerenic acid) | |
| | Volatiles/gases (e.g., chloral | |
| | hydrate, chloroform, diethyl ether, | |
| | sevoflurane) | |
| Glutamate Receptor | 3,5-dihydroxyphenylglycine | APICA |
| Accession Number: | eglumegad | EGLU |
| (P42261, P39086, | Biphenylindanone A | LY-341,495 |
| P39086, Q13585, P42261, | DCG-IV | |
| P42262, P42263, P48058, | L-AP4 | |
| P39086, Q13002, | | |
| Q13003, Q13003, | | |
| Q16478, Q12879, | | |
| Q14957, Q13224, | | |
| Q14957, Q15399, | | |
| Q8TCU5, O60391) | | |
| CNR1/CNR2 | N-Arachidonoylethanolamine | SR 141716A |
| Accession Number: | 2-Arachidonoyl-glycerol | LY-320135 |
| (P21554, P34972) | 2-Arachidonoyl-glycerylether | AM251 |
| | N-Arachidonoyl-dopamine | AM281 |
| | O-Arachidonoyl-ethanolamine | SR 144528 |
| | N-Arachidonoylethanolamine | AM630 |
| | 2-Arachidonoyl-glycerol | |
| | 2-Arachidonoyl-glycerylether | |
| | N-Arachidonoyl-dopamine | |
| | O-Arachidonoyl-ethanolamine | |
| | Δ9-THC | |
| | CP-55,940 | |
| | R(+)-WIN 55,212-2 | |
| | HU-210 | |
| | Levonantradol | |
| | Nabilone | |
| | Methanandamide | |
| | ACEA | |
| | O-1812 | |
| | Δ9-THC | |
| | CP-55,940 | |
| | R(+)-WIN 55,212-2 | |
| | HU-210 | |
| | Levonantradol | |
| | Nabilone | |
| | Methanandamide | |
| | JWH-015 | |
| | JWH-133 | |

TABLE 7B
ADRENERGIC AGONISTS AND ANTAGONISTS

| Receptor | Agonist | Antagonist |
|---|---|---|
| Non-selective | adrenaline (epinephrine), | carvedilol, arotinolol, and labetalol |
| | noradrenaline (norepinephrine), | |
| | isoprenaline (isoproterenol), | |
| | dopamine, caffeine, nicotine, | |
| | tyramine, methylphenidate, ephedrine | |
| | and pseudophedrine. | |
| α1 selective (ADRA1A, | phenylephrine, methoxamine, | acepromazine, alfuzosin, doxazosin, |
| ADRA1B, ADRA1D) | midodrine, cirazoline, | labetalol, phenoxybenzamine, |
| | xylometazoline, metaraminol | KW3902, phentolamine, prazosin, |
| | chloroehtylclonidine, oxymetazoline | tamsulosin, terazosin, tolazoline, |
| | | trazodone, amitriptyline, silodosin, |
| | | clomipramine, doxepin, trimipramine, |
| | | typical and atypical antipsychotics, and |
| | | antihistamines, such as hyroxyzine |
| α2 selective (ADRA2A, | α-methyl dopa, clonidine, | phentolamine, phenoxybenzamine, |
| ADRA2B, ADRA2C) | brimonidine, agmatine, | yohimbine, idazoxan, atipamezole, |
| | dexmedetomidine, | mirtazapine, tolazoline, trazodone, and |
| | medetomidine, romifidine | typical and atypical antipsychotics |
| | chloroethylclonidine, | |
| | detomidine, lofexidine, xylazine, | |
| | tizanidine, guanfacine, and amitraz | |
| β1 selective (ADRB1) | Dobutamine | metroprolol, atenolol, acebutolol, |
| | | bisoprolol, betaxolol, levobetaxolol, |
| | | esmolol, celiprolol, carteolol, landiolol, |
| | | oxprenolol, propanolol, practolol, |
| | | penbutolol, timolol, labetalol, nebivolol, |
| | | levobunolol, nadolol, pindolol, sotalol, |
| | | metipranolol, tertatolol, vortioxene |
| β2 selective (ADRB2) | salbutamol, albuterol, bitolterol | butaxamine, acebutolol, timolol, |
| | mesylate, levabuterol, ritodrine, | propanolol, levobunolol, carteolol, |
| | metaproterenol, terbutaline, | labetalol, pindolol, oxprenolol, nadolol, |
| | salmeterol, formoterol, and pirbuterol | metipranolol, penbutolol, tertatolol, |
| | | sotalol |
| β3 selective (ADRB3) | L-796568, amibegron, solabegron, | SR 59230A, arotinolol |
| | mirabegron | |

TABLE 7C
DOPAMINE AGONISTS AND ANTAGONISTS

| Receptor | Agonist | Antagonist |
|---|---|---|
| Non-selective | pramipexole, ropinirole, rotigotine, | haloperidol, paliperidone, clozapine, |
| | apomorphine, | risperidone, olanzapine, quetiapine, |
| | propylnorapomorphine, | ziprasidone, metoclopramide, |
| | bromocriptine, cabergoline, | droperidol, domperidone, amoxapine, |
| | ciladopa, dihydrexidine, | clomipramine, trimipramine, choline, |
| | dinapsoline, doxamthrine, | melatonin, acepromazine, amisulpride, |
| | epicriptine, lisuride, pergolide, | asenapine, azaperone, benperidol, |
| | piribedil, quinagolide, roxindole, | bromopride, butaclamol, |
| | dopamine | chlorpromazine, clebopride, |
| | | chlorprothixene, clopenthixol, |
| | | clocapramine, eticlopride, flupenthixol, |
| | | fluphenazine, fluspirilene, hydroxyzine, |
| | | itopride, iodobenzamide, |
| | | levomepromazine, levosulpiride, |
| | | loxapine, mesoridazine, metopimazine, |
| | | mosapramine, nafadotride, |
| | | nemonapride, penfluridol, perazine, |
| | | perphenazine, pimozide, |
| | | prochlorperazine, promazine, |
| | | pipotiazine, raclopride, remoxipride, |
| | | spiperone, spiroxatrine, stepholidine, |
| | | sulpiride, sultopride, |
| | | tetrahydropalmatine, thiethylperazine, |
| | | thioridazine, thiothixene, tiapride, |
| | | trifluoperazine, trifluperidol, |
| | | triflupromazine, thioproperazine, |
| | | taractan, zotepine, zuclopenthixol, |
| | | ziprasidone, ANP-010, NGD-94-4 |
| D1 (DRD1) | Fenoldopam, A-86929, | SCH-23,390, SKF-83,959, Ecopipam, |
| | dihydrexidine, dinapsoline, | Clebopride, Flupenthixol, |
| | dinoxyline, doxanthrine, SKF- | Zuclopenthixol, Taractan, PSYRX-101, |
| | 81297, SKF-82958, SKF-38393, G- | LuAF-35700, GLC-756, ADX10061, |
| | BR-APB, dopexamine | Zicronapine |
| D2 (DRD2) | Cabergoline, pergolide, | Chloroethylnorapomorphine, |
| | quinelorane, sumanirole, talipexole, | desmethoxyfallypride, domperidone, |
| | piribedil, quinpirole, quinelorane, | eticlopride, fallypride, hydroxyzine, |
| | dinoxyline, dopexamine | itopride, L-741,626, SV 293, yohimbine, |
| | | raclopride, sulpiride, paliperidone, |
| | | penfluridol, quetiapine, lurasidone, |
| | | risperidone, olanzapine, blonanserin, |
| | | perphenazine, metoclopramide, |
| | | trifluoperazine, clebopride, levosulpiride, |
| | | flupenthixol, haloperidol, thioridazine, |
| | | alizapride, amisulpride, asenapine, |
| | | bromopride, bromperidol, clozapine, |
| | | fluphenazine, perphanazine, loxapine, |
| | | nemonapride, pericyazine, |
| | | pipamperone, prochlorperazine, |
| | | thioproperazine, thiethylperazine, |
| | | tiapride, ziprasidone, zuclopenthixol, |
| | | taractan, fluanisone, melperone, |
| | | molindone, remoxipride, sultopride, |
| | | ALKS 3831, APD-403, ONC201, |
| | | pridopidine, DSP-1200, NG-101, TAK- |
| | | 906, ADN-1184, ADN-2013, AG-0098, |
| | | DDD-016, IRL-626, KP303, ONC-206, |
| | | PF-4363467, PGW-5, CG-209, ABT- |
| | | 925, AC90222, ACP-005, ADN-2157, |
| | | CB030006, CLR-136, Egis-11150, |
| | | Iloperidone, JNJ-37822681, DLP-115, |
| | | AZ-001, S-33138, SLV-314, Y-931, |
| | | YKP1358, YK-P1447, APD405, CP- |
| | | 903397, ocaperidone, zicronapine, |
| | | TPN-902 |
| D3 (DRD3) | Piribedil, quinpirole, captodiame, | Domperidone, FAUC 365, nafadotride, |
| | compound R, R-16, FAUC 54, | raclopride, PNU-99,194, SB-277011-A, |
| | FAUC 73, PD-128,907, PF- | sulpiride, risperidone, YQA14, U99194, |
| | 219,061, PF-592,379, CJ-1037, | SR 21502, levosulpiride, amisulpride, |
| | FAUC 460, FAUC 346, cariprazine | nemonapride, ziprasidone, taractan, |
| | | sultopride, APD-403, F17464, ONC201, |
| | | NG-101, TAK-906, ONC-206, PF- |
| | | 4363467, ABT-127, ABT-614, GSK- |
| | | 598809, GSK-618334, S-14297, S- |
| | | 33138, YKP1358, YK-P1447 |
| D4 (DRD4) | WAY-100635, A-412,997, ABT-724, | A-381393, FAUC 213, L-745,870, L- |
| | ABT-670, FAUC 316, PD-168,077, | 570,667, ML-398, fananserin, clozapine, |
| | CP-226,269 | PNB-05, SPI-376, SPI-392, Lu-35-138, |
| | | NGD-94-1 |
| D5 (DRD5) | Dihydrexidine, rotigotine, SKF- | SCH 23390 |
| | 83,959, fenoldopam, | |
| Partial | aplindore, brexpiprazole, | |
| | aripiprazole, CY-208,243, | |
| | pardoprunox, phencyclidine, and | |
| | salvinorin A | |

TABLE 7D
GABA AGONISTS AND ANTAGONISTS

| Receptor | Agonist | Antagonist |
|---|---|---|
| GABA_{A} | barbiturates (e.g., allobarbital, | bicuculline, gabazine, hydrastine, |
| | amobarbital, aprobarbital, alphenal, | pitrazepin, sinomenine, tutin, |
| | barbital, brallobarbital, | thiocolchicoside, metrazol, securinine, |
| | phenobarbital, secobarbital, | gabazine |
| | thiopental), bamaluzole, GABA, | |
| | GABOB, gaboxadol, ibotenic acid, | |
| | isoguvacine, isonipecotic acid, | |
| | muscimol, phenibut, picamilon, | |
| | progabide, quisqualamine, SL | |
| | 75102, thiomuscimol, positive | |
| | allosteric modulators (PAMs) (e.g., | |
| | alcohols, such as ethanol and | |
| | isopropanol; avermectins, such as | |
| | ivermectin; benzodiazepines, such | |
| | as diazepam, alprazolam, | |
| | chlordiazepoxide, clonazepam, | |
| | flunitrazepam, lorazepam, | |
| | midazolam, oxazepam, prazepam, | |
| | brotizolam, triazolam, estazolam, | |
| | lormetazepam, nitrazepam, | |
| | temazepam, flurazepam, | |
| | clorazepate halazepam, prazepam, | |
| | nimetazapem, adinazolam, and | |
| | climazolam; bromides, such as | |
| | potassium bromide; carbamates, | |
| | such as meprobamate and | |
| | carisoprodol; chloralose; | |
| | chlormezanone; chlomethiazole; | |
| | dihydroergolines, such as ergoloid; | |
| | etazepine; etifoxine; imidazoles, | |
| | such as etomidate; | |
| | imidazopyridines, such as alpidem | |
| | and necopdiem; kavalactones; | |
| | loreclezole; neuroactive steroids, | |
| | such as allogregnanolone, | |
| | pregnanolone, | |
| | dihydrodeoxycorticosterone, | |
| | tetrahydrodeoxycortisosterone, | |
| | androstenol, androsterone, | |
| | etiocholanolone, 3α-androstanediol, | |
| | 5α, 5β, or 3α-dihydroprogesterone, | |
| | and ganaxolone; | |
| | nonbenzodiazepines, such as | |
| | zalepon, zolpidem, zopiclone, and | |
| | eszopiclone; petrichloral; phenols, | |
| | such as propofol; piperidinediones, | |
| | such as glutethimide and | |
| | methyprylon; propanidid; | |
| | pyrazolopyridines, such as | |
| | etazolate; pyrazolopyrimidines, | |
| | such as divaplon and fasiplon; | |
| | cyclopyrrolones, sush as pagoclone | |
| | and suproclone; β-cabolines, such | |
| | as abecarnil and geodecarnil; | |
| | quinazolinones, such as | |
| | methaqualone; Scutellaria | |
| | constituents; stiripentol; | |
| | sulfonylalkanes, such as | |
| | sulfonomethane, teronal, and | |
| | trional; Valerian constituents, such | |
| | as valeric acid and valerenic acid; | |
| | and gases, such as chloral hydrate, | |
| | chloroform, homotaurine, diethyl | |
| | ether, and sevoflurane. | |
| GABA_{B} | 1,4-butanediol, baclofen, GABA, | CGP-35348, homotaurine, phaclofen, |
| | Gabamide, GABOB, gamma- | saclofen, and SCH-50911 |
| | butyrolactone, gamma- | |
| | hydroxybutyric acid, gamma- | |
| | hyrdoxyvaleric acid, gamma- | |
| | valerolactone, isovaline, | |
| | lesogaberan, phenibut, picamilon, | |
| | progabide, homotaurine, SL-75102, | |
| | tolgabide | |
| GABA_{A}-ρ | CACA, CAMP, GABA, GABOB, N4- | gabazine, gaboxadol, isonipecotic acid, |
| | chloroacetylcytosine arabinoside, | SKF-97,541, and (1,2,5,6- |
| | picamilon, progabide, tolgabide, | Tetrahydropyridin-4-yl)methylphosphinic |
| | and neuroactive steroids, such as | acid |
| | allopregnanolone, THDOC, and | |
| | alphaxolone | |

TABLE 7E
MUSCARINC AGONISTS AND ANTAGONISTS

| Receptor | Agonist | Antagonist |
|---|---|---|
| Chrm1 | AF102B, AF150(S), AF267B, | atropine, dicycloverine, hyoscyamine, |
| | acetylcholine, carbachol, cevimeline, | ipratropium, mamba toxin muscarinic |
| | muscarine, oxotremorine, pilocarpine, | toxin 7 (MT7), olanzapine, oxybutynin, |
| | vedaclidine, 77-LH-28-1, CDD-0097, | pirenzepine, telenzepine, and |
| | McN-A-343, L689,660, and | tolterodine |
| | xanomeline | |
| Chrm2 | acetylcholine, methacholine, iper-8- | atropine, dicycloverine, hyoscyamine, |
| | naph, berbine, and (2S,2′R,3′S,5′R)- | otenzepad, AQRA-741, AFDX-384, |
| | 1-methyl-2-(2-methyl-1,3-oxathiolan- | thorazine, diphenhydramine, |
| | 5-yl)pyrrolidine 3-sulfoxide methyl | dimenhydrinate, ipratropium, |
| | iodide | oxybutynin, pirenzepine, |
| | | methoctramine, tripitramine, |
| | | gallamine, and tolterodine |
| Chrm3 | acetylcholine, bethanechol, | atropine, dicycloverine, hyoscyamine, |
| | carbachol, L689, 660, oxotremorine, | alcidium bromide, 4-DAMP, |
| | pilocarpine, aceclidine, arecoline, | darifenacin, DAU-5884, HL-031,120, |
| | and cevimeline | ipratropium, J-104,129, oxybutynin, |
| | | tiotropium, zamifenacin, and |
| | | tolterodine |
| Chrm4 | acetylcholine, carbachol, and | AFDX-384, dicycloverine, himbacine, |
| | oxotremorine), and Chrm5 agonists | mamba toxin 3, PD-102,807, |
| | (e.g., acetylcholine, milameline, | PD-0298029, and tropicamide |
| | sabcomeline | |
| Chrm5 | acetylcholine, milameline, | VU-0488130, xanomeline |
| | sabcomeline | |
| Non-selective | | scopolamine, hydroxyzine, |
| | | doxylamine, dicyclomine, flavoxate, |
| | | cyclopentolate, atropine methonitrate, |
| | | trihexyphenidyl/benzhexol, |
| | | solifenacin, benzatropine, |
| | | mebeverine, and procyclidine |

TABLE 7F
SEROTONIN AGONISTS AND ANTAGONISTS

| Receptor | Agonist | Antagonist |
|---|---|---|
| 5-HT_{1A} | azapirones, such as alnespirone, | pindolol, tertatolol, alprenolol, AV- |
| | binosperone, buspirone, | 965, BMY-7,378, cyanopindolol, |
| | enilospirone, etapirone, geprione, | dotarizine, flopropione, GR-46,611, |
| | ipsaprione, revospirone, zalospirone, | iodocyanopindolol, isamoltane, |
| | perospirone, tiosperone, | lecozotan, mefway, methiothepin, |
| | umespirone, and tandospirone; 8- | methysergide, MPPF, NAN-190, |
| | OH-DPAT, befiradol, F-15,599, | oxprenolol, pindobind, propanolol, |
| | lesopitron, MKC-242, LY-283,284, | risperidone, robalzotan, SB-649,915, |
| | osemozotan, repinotan U-92,016-A, | SDZ-216,525, spiperone, spiramide, |
| | RU-24969, 2C-B, 2C-E, 2C-T-2, | spiroxatrine, UH-301, WAY-100,135, |
| | aripiprazole, asenapine, bacoside, | WAY-100,635, and xylamidine |
| | befiradol, brexpiprazole, bufotenin, | |
| | cannabidiol, and fibanserin | |
| 5-HT_{1B} | triptans, such as sumatriptan, | methiothepin, yohimbine, |
| | rizatriptan, eletriptan, donitripatn, | metergoline, aripiprazole, isamoltane, |
| | almotriptan, frovatriptan, avitriptan, | AR-A000002, SB-216,641, SB- |
| | zolmitriptan, and naratriptan; | 224,289, GR-127,935, SB-236,057 |
| | ergotamine, 5- | |
| | carboxamidotryptamine, CGS- | |
| | 12066A, CP-93,129, CP-94,253, | |
| | CP-122,288, CP-135,807, RU-24969, | |
| | vortioxetine, ziprasidone, and | |
| | asenapine | |
| 5-HT_{1D} | triptans, such as sumatriptan, | ziprasidone, methiothepin, |
| | rizatriptan, and naratriptan; | yohimbine, metergoline, ergotamine, |
| | ergotamine, 5-(nonyloxy)tryptaime, | BRL-15572, vortioxetine, |
| | 5-(t-butyl)-N-methyltryptamine, | GR-127,935, LY-310,762, LY-367,642, |
| | CP-286,601, PNU-109,291, PNU- | LY-456,219, and LY-456,220 |
| | 142,633, GR-46611, L-694,247, | |
| | L-772,405, CP-122,288, | |
| | and CP-135,807 | |
| 5-HT_{1E} | BRL-54443, eletriptan | |
| 5-HT_{1F} | LY-334,370, 5-n-butyryloxy-DMT, | |
| | BRL-54443, eletriptan, LY-344,864, | |
| | naratriptan, and lasmiditan | |
| 5-HT_{2A} | 25I-NBOH, 25I-NBOMe, (R)-DOI, | cyproheptadine, methysergide, |
| | TCB-2, mexamine, O-4310, PHA- | quetiapine, nefazodone, olanzapine, |
| | 57378, OSU-6162, 25CN-NBOH, | asenapine, pizotifen, LY-367,265, |
| | juncosamine, efavirenz, mefloquine, | AMDA, hydroxyzine, 5-MeO-NBpBrT, |
| | lisuride, and 2C-B | and niaprazine |
| 5-HT_{2B} | fenfluramine, pergolide, cabergoline, | agomelatine, aripiprazole, |
| | mefloquine, BW-723086, Ro60- | sarpogrelate, lisuride, tegaserod, |
| | 0175, VER-3323, 6-APB, | metadoxine, RS-127,445, SDZ SER- |
| | guanfacine, norfenfluramine, 5-MeO- | 082, EGIS-7625, PRX-08066, SB- |
| | DMT, DMT, mCPP, aminorex, | 200,646, SB-204,741, SB-206,553, |
| | chlorphentermine, MEM, MDA, LSD, | SB-215,505, SB-228,357, LY- |
| | psilocin, MDMA | 266,097, and LY-272,015 |
| 5-HT_{2C} | lorcaserin, lisuride, A-372,159, AL- | agomelatine, CPC, eltoprazine, |
| | 38022A, CP-809,101, fenfluramine, | etoperidone, fluoxetine, FR-260,010, |
| | mesulergine, MK-212, | LU AA24530, methysergide, |
| | naphthyllisopropylamine, | nefazodone, norfluoxetine, |
| | norfenfluramine, ORG-12,962, ORG- | O-desmethyltramadol, RS-102,221, |
| | 37,684, oxaflozane, PNU-22395, | SB-200,646, SB-221,284, SB-242,084, |
| | PNU-181731, lysergamides, | SDZ SER-082, tramadol, and |
| | phenethylamines, piperazines, | trazodone |
| | tryptamines, Ro60-0175, | |
| | vabicaserin, WAY-629, | |
| | WAY-161,503, | |
| | WAY-163,909, and YM-348 | |
| 5-HT_{2A/2C} | | ketanserin, risperidone, trazodone, |
| | | mirtazapine, clozapine |
| 5-HT₃ | 2-methyl-5-HT, alpha- | dolasetron, granisetron, ondansetron, |
| | methyltryptamine, bufotenin, | palonosetron, tropisetron, alosetron, |
| | chlorophenylbiguanide, ethanol, | cilanosetron, mirtazapine, AS-8112, |
| | ibogaine, phenylbiguanide, | bantopride, metroclopramide, |
| | quipazine, RS-56812, SR-57227, | renzapride, zacopride, mianserin, |
| | varenicline, and YM-31636 | vortioxetine, clozapine, olanzapine, |
| | | quetiapine, menthol, thujone, |
| | | lamotigrine, and 3-tropanyl |
| | | indole-3-carboxylate |
| 5-HT₄ | cisapride, tegaserod, prucalopride, | piboserod, GR-113,808, GR- |
| | BIMU-8, CJ-033,466, ML-10302, | 125,487, RS-39604, SB-203,186, |
| | mosapride, renzapride, RS-67506, | SB-204,070, and chamomile |
| | RS-67333, SL65.1055, zacopride, | |
| | metoclopramide, and sulpride | |
| 5-HT_{5A} | valeronic acid | ASP-5736, AS-2030680, |
| | | AS-2674723, latrepiridine, |
| | | risperidone, |
| | | and SB-699,551 |
| 5-HT₆ | EMDT, WAY-181,187, WAY- | ALX-1161, AVN-211, BVT-5182, |
| | 208,466, N-(inden-5- | BVT-74316, cerlapiridine, EGIS- |
| | yl)imidazothiazole- | 12233, idalopiridine, interpridine, |
| | 5-sulfonamide, | latrepiridine, MS-245, PRX-07034, |
| | E-6837, E-6801, and | SB-258,585, SB-271,046, SB- |
| | EMD-386,088 | 357,134, SB-339,885, Ro 04-6790, |
| | | Ro-4368554, sertindole, olanzapine, |
| | | asenapine, clozapine, rosa rugosa |
| | | extract, and WAY-255315 |
| 5-HT₇ | AS-19, 5-CT, 5-MeOT, | amisulpride, amitriptyline, |
| | 8-OH-DAPT, | amoxapine, clomipramine, clozapine, |
| | aripiprazole, E-55888, | DR-4485, fluphenazine, fluperlapine, |
| | E-57431, LP-12, | ICI 169,369, imipramine, ketanserine, |
| | LP-44, MSD-5a, RA-7, and | JNJ-18038683, loxapine, lurasidone, |
| | N,N-Dimethyltryptamine | LY-215,840, maprotiline, |
| | | methysergide, mesulergine, |
| | | mianserin, olanzepine, pimozide, |
| | | ritanserin, SB-258,719, SB-258,741, |
| | | SB-269,970, SB-656,104-A, SB- |
| | | 691,673, sertindole, spiperone, |
| | | tenilapine, TFMPP, vortioxetine, |
| | | trifluoperazine, ziprasidone, |
| | | and zotepine |
| Non-selective | | chlorpromazine, cyproheptadine, |
| 5-HT | | pizotifen, oxetorone, spiperone, |
| antagonists | | ritanserin, parachlorophenylalanine, |
| | | metergoline, propranolol, mianserin, |
| | | carbinoxamine, methdilazine, |
| | | promethazine, pizotifen, oxatomide, |
| | | feverfew, fenclonin, and reserpine |

TABLE 7G
GLUATAMATE RECEPTOR AGONISTS AND ANTAGONISTS

| Receptor | Agonist | Antagonist |
|---|---|---|
| Ionotropic | AMPA, glutamic acid, | AP5, AP7, CPPene, selfotel, |
| (GRIA-14, | ibotenic acid, | HU-211, Huperzine A, |
| GRIK1-5, | kainic acid, | gabapentin, |
| and | NMDA, | remacemide, amantadine, |
| GRIN1-3B) | quisqualic acid | atomoxetine, AZD6765, |
| | | agmatine, chloroform, |
| | | dextrallorphan, |
| | | dextromethorphan, |
| | | dextrorphan, |
| | | diphenidine, |
| | | dizocilpine (MK-801), |
| | | ethanol, eticyclidine, |
| | | gacyclidine, |
| | | ibogaine, ifenprodil, |
| | | ketamine, kynurenic |
| | | acid, memantine, |
| | | magnesium, methoxetamine, |
| | | nitromemantine, nitrous |
| | | oxide, PD-137889, |
| | | perampanel, phencyclidine, |
| | | rolicyclidine, tenocyclidine, |
| | | methoxydine, tiletamine, |
| | | neramexane, eliprodil, |
| | | etoxadrol, |
| | | dexoxadrol, WMS-2539, |
| | | NEFA, delucemine, |
| | | 8A-PDHQ, aptiganel, |
| | | rhynchophylline |
| Metabotropic | L-AP4, ACPD, L-QA, | AIDA, fenobam, MPEP, |
| (GRM1-8) | CHPG, LY-379,268, | LY-367,385, EGLU, |
| | LY-354,740, ACPT, | CPPG, MAP4, MSOP, |
| | VU0155041 | LY-341,495 |
| Glycine | | rapastinel, NRX-1074, 7- |
| antagonists | | chlorokynurenic acid, 4- |
| | | chlorokynurenine, 5,7- |
| | | dichlorokynurenic acid, |
| | | kynurenic acid, TK-40, |
| | | 1-aminocyclopropanecarboxylic |
| | | acid (ACPC), |
| | | L-phenylalanine, and xenon |

TABLE 7H
HISTAMINE AGONISTS AND ANTAGONISTS

| Receptor | Agonist | Antagonist |
|---|---|---|
| Non-selective | histamine dihydrochloride, HTMT | |
| | dimaleate, 2-pyridylethlyamine | |
| | dihydrochloride | |
| H₁ | | acrivastine, azelastine, astemizole, |
| | | bilastine, bromodiphenhydramine, |
| | | brompheniramine, buclizine, |
| | | carbinoxamine, cetirizine, cetirizine |
| | | dihydrochloride, clemastine fumarate, |
| | | clemizole hydrochloride, |
| | | chlorodiphenhydramine, |
| | | chlorphenamine, chlorpromazine, |
| | | clemastine, cyclizine, cyproheptadine, |
| | | dexbrompheniramine, |
| | | dexchlorpheniramine, dimenhydrinate, |
| | | dimethindene maleate, dimetindene, |
| | | diphenhydramine, diphenhydramine |
| | | hydrochloride, doxepin hydrochloride, |
| | | doxylamine, ebastine, embramine, |
| | | fexofenadine, fexofenadine |
| | | hydrochloride, hydroxyzine, ketotifen |
| | | fumarate, loratadine, meclizine, |
| | | meclizine dihydrochloride, |
| | | mepyramine maleate, mirtazapine, |
| | | olopatadine, olopatadine |
| | | hydrochloride, orphenadrine, |
| | | phenindamine, pheniramine, |
| | | phenyltoloxamine, promethazine, |
| | | quetiapine, rupatadine, terfenadine, |
| | | tripelennamine, zotepine, trans- |
| | | triprolidine hydrochloride, and |
| | | triprolidine |
| H₁ inverse | | cetirizine, levocetirizine, |
| agonists | | desloratadine, and pyrilamine |
| H₂ | betazole, impromidine, dimaprit | aminopotentidine, cimetidine, |
| | dihydrochloride, and amthamine | famotidine, ICI 162,846, lafutidine, |
| | dihyrdobromide | nizatidine, ranitidine, ranitidine |
| | | hyrdochloride, roxatidine, zolantadine |
| | | dimaleate, and toitidine |
| H₃ | imetit dihydropbromide, immepip | clobenpropit, clobenpropit |
| | dihyrdrobromide, immethridine | dihydrobromide, A 3314440 |
| | dihydrobromide, | dihyrdochloride, BF 2649 |
| | α-Methylhistamine | hydrochloride, carcinine |
| | dihydrobromide, | ditrifluoroacetate, ABT-239, ciprofaxin, |
| | N-methylhistamine | conessine, GT 2016, A-349,821, |
| | dihydrochloride, | impentamine dihydrobromide, |
| | proxyfan oxalate, | iodophenpropit dihydrobromide, JNJ |
| | and betahistine | 10181457 dihydrochloride, JNJ |
| | | 5207852 dihydrochloride, ROS 234 |
| | | dioxalate, SEN 12333, VUF 5681 |
| | | dihydrobromide, and thioperamide |
| H₄ | imetit dihydropbromide, immepip | thioperamide, JNJ 7777120, A 943931 |
| | dihyrdrobromide, 4-methylhistamine | dihydrochloride, A 987306, JNJ |
| | dihydrochloride, clobenpropit | 10191584 maleate, and VUF-6002 |
| | dihydrobromide, VUF 10460, and | |
| | VUF 8430 dihydrobromide | |

TABLE 7I
CANNABINOID AGONISTS AND ANTAGONISTS

| Receptor | Agonist | Antagonist |
|---|---|---|
| Cannabinoid receptor | Anandamide, N-Arachidonoyl | |
| (non-selective) | dopamine, 2-Arachidonoylglycerol | |
| | (2-AG), 2-Arachidonyl glyceryl | |
| | ether, Δ-9-Tetrahydrocannabinol, | |
| | EGCG, Yangonin, AM-1221, AM- | |
| | 1235, AM-2232, UR-144, JWH- | |
| | 007, JWH-015, JWH-018, ACEA, | |
| | ACPA, arvanil, CP 47497, DEA, | |
| | leelamine, methanandamide, | |
| | NADA, noladin ether, oleamide, | |
| | CB 65, GP-1a, GP-2a, GW | |
| | 405833, HU 308, JWH-133, L- | |
| | 759,633, L-759,656, LEI 101, MDA | |
| | 19, and SER 601 | |
| CB₁ receptor | ACEA, ACPA, RVD-Hpα, (R)-(+)- | rimonabant, cannabidiol, Δ⁹- |
| | methanandamide | tetrahydrocannabivarin (THCV), |
| | | taranabant, otenabant, surinabant, |
| | | rosonabant, SLV-319, AVE1625, |
| | | V24343, AM 251, AM 281, AM 6545, |
| | | hemopressin, LY 320135, MJ 15, CP |
| | | 945598, NIDA 41020, PF 514273, SLV |
| | | 319, SR 1141716A, and TC-C 14G |
| CB₂ receptor | CB 65, GP 1a, GP 2a, GW | cannabidiol, Δ⁹-tetrahydrocannabivarin |
| | 405833, HU 308, JWH 133, L- | (THCV), AM 630, COR 170, JTE 907, |
| | 759,656, L-759,633, SER 601, LEI | and SR 144528 |
| | 101 | |

TABLE 7J
PURINERGIC RECEPTOR AGONISTS AND ANTAGONISTS

| Receptor | Agonist | Antagonist |
|---|---|---|
| ADORA1 (P1 | Adenosine, N6- | Caffeine, theophylline, 8-Cyclopentyl- |
| adenosine receptor) | Cyclopentyladenosine, N6-3- | 1,3-dimethylxanthine (CPX), 8- |
| | methoxyl-4-hydroxybenzyl adenine | Cyclopentyl-1,3-dipropylxanthine |
| | riboside (B2), CCPA, tecadenoson, | (DPCPX), 8-Phenyl-1,3- |
| | selodenoson, Certain | dipropylxanthine, bamifylline, BG- |
| | Benzodiazepines and Barbiturates, | 9719, BG09928, FK-453, FK838, |
| | 2′-MeCCPA, GR 79236, and SDZ | rolofylline, N-0861, and PSB 36 |
| | WAG 994 | |
| ADORA2A (P1 | Adenosine, N6-3-methoxyl-4- | Caffeine, theophylline, istradefylline, |
| adenosine receptor) | hydroxybenzyl adenine riboside | SCH-58261, SCH-442,416, ATL-444, |
| | (B2), YT-146, DPMA, UK-423,097, | MSX-3, preladenant, SCH-412,348, |
| | limonene, NECA, CV-3146, | VER-6623, VER-6947, VER-7835, |
| | binodenoson, ATL-146e, CGS- | vipadenant, and ZM-241,385 |
| | 21680, and Regadenoson | |
| ADORA2B (P1 | Adenosine, 5′-N- | Caffeine, theophylline, CVT-6883, |
| adenosine receptor) | ethylcarboxamidoadenosine, BAY | ATL-801, compound 38, MRS-1706, |
| | 60-6583, LUF-5835, NECA, (S)- | MRS-1754, OSIP-339,391, PSB-603, |
| | PHPNECA, and LUF-5845 | PSB-0788, and PSB-1115 |
| ADORA3 (P1 | Adenosine, 2-(1-Hexynyl)-N- | Caffeine, theophylline, MRS-1191, |
| adenosine receptor) | methyladenosine, CF-101 (IB- | MRS-1220, MRS-1334, MRS-1523, |
| | MECA), CF-102, 2-CI-IB-MECA, | MRS-3777, MRE3008F20, |
| | CP-532,903, inosine, LUF-6000, | MRE3005F20, OT-7999, SSR161421, |
| | and MRS-3558 | KF-26777, PSB-10, PSB-11, and VUF- |
| | | 5574 |
| P2Y receptor | ATP, ADP, UTP, UDP, UDP- | clopidogrel, elinogrel, prasugrel, |
| | glucose, 2-methylthioladenosine 5′ | ticlopidine, ticagrelor, AR-C |
| | diphosphate (2-MeSADP), | 118925XX, AR-C 66096, AR-C 69931, |
| | lysophosphatidic acid, PSB 1114, | AZD 1283, MRS 2179, MRS 2211, |
| | PSB 0474, NF 546, MRS 2365, | MRS 2279, MRS 2500, MRS 2578, NF |
| | MRS 2690, MRS 2693, MRS 2768, | 157, NF 340, PPADS, PPTN |
| | MRS 2905, MRS 2957, MRS 4062, | hydrochloride, PSD 0739, SAR |
| | and denufosol (P2Y₂ agonist) | 216471, and suramin |
| P2X receptor | ATP | A 438079, A 740003, A 804598, A |
| | | 839977, AZ 10606120, AZ 11645373, |
| | | 5-BDBD, BX 430, Evans Blue, JNJ |
| | | 47965567, KN-62, NF 023, NF 110, |
| | | NF 157, NF 279, NF 449, PPADS, iso- |
| | | PPADS, PPNDS, Ro 0437626, Ro 51, |
| | | RO-3, TC-P 262, suramin, TNP-ATP, |
| | | and P2X₇ antagonists NF279, |
| | | calmidazolium, and KN-62 |

TABLE 8
NEUROTRANSMISSION MODULATORS

| Type | Modulators |
|---|---|
| Norepinephrine reuptake inhibitors | amedalin, atomoxetine, CP-39,332, daledalin, |
| (increase adrenergic neurotransmission) | edivoxetine, esreboxetine, lortalamine, nisoxetine, |
| | reboxetine, talopram, talsupram, tandamine, |
| | viloxazine, bupropion, ciclazindol, manifaxine, |
| | maprotiline, radafaxine, tapentadol, teniloxazine, |
| | protriptyline, nortriptyline, and desipramine |
| Norepineprhine-dopamine reuptake inhibitors | amineptine, bupropion, desoxypipradrol, |
| (increase adrenergic and dopamine | dexmethylphenidate, difemetorex, diphenylprolinol, |
| neurotransmission) | ethylphenidate, fencamfamine, fencamine, |
| | lefetamine, methylenedioxypyrovalerone, |
| | methylphenidate, nomifensine, O-2172, oxolinic |
| | acid, pipradrol, prolintane, pyrovalerone, |
| | tametraline, and WY-46824 |
| Serotonin-norepinephrine-dopamine reuptake | mazindol, nefazodone, sibutramine, venlafaxine, |
| inhibitors (SNDRIs) and serotonin-norepinephrine | esketamine, duloxetine, ketamine, phencyclidine, |
| reuptake inhibitors (SNRIs) | tripelennamine, mepiprazole, amitifadine, AN788, |
| (increase adrengergic, dopamine, and serotonin | ansofaxine, centanafadine, atomoxetine, |
| neurotransmission) | desvenlafaxine, milnacipran, levomilnacipran, |
| | dasotraline, Lu AA34893, Lu AA37096, NS-2360, |
| | tedatioxetine, tesofensine, bicifadine, BMS- |
| | 866,949, brasofensine, diclofensine, DOV-216,303, |
| | EXP-561, liafensine, NS-2359, RG-7166, SEP- |
| | 227,162, SEP-228,425, SEP-228,432, naphyrone, |
| | 3,3-Diphenylcyclobutanamine, 3,4- |
| | Dichlorotametraline, D-161, desmethylsertraline, |
| | DMNPC, DOV-102,677, fezolamine, |
| | GSK1360707F, indatraline, JNJ-7925476, JZ-IV- |
| | 10, JZAD-IV-22, LR-5182, methylnaphthidate, MI-4, |
| | PRC200-SS, PRC050, PRC025, SKF-83,959, TP1, |
| | phenyltropanes (e.g., WF-23, dichloropane, and |
| | RTI-55), Ginkgo biloba extract, St John's Wort, |
| | hyperforin, adhyperforin, and uliginosin B |
| Dopamine reuptake inhibitors | Dopamine reuptake inhbiitors (e.g., altropane, |
| (increase dopamine neurotransmission) | amfonelic acid, amineptine, BTCP, 3C-PEP, DBL- |
| | 583, difluoropine, GBR-12783, GBR-12935, GBR- |
| | 13069, GBR-13098, GYKI-52895, lometopane, |
| | methylphenidate, ethylphenidate, modafinil, |
| | armodafinil, RTI-229, vanoxerine, adrafinil, |
| | benztropine, bupropion, fluorenol, medifoxamine, |
| | metaphit, rimcazole, venlafaxine, Chaenomeles |
| | speciosa, and oroxylin A), dopamine releasing |
| | agents (e.g., p-Tyramine), dextroamphetamine, |
| | lisdexamfetamine, dexmethylphenidate, and |
| | cathinone |
| Dopamine prodrugs | Levopoda, docarpamine |
| (increase dopamine neurotransmission) | |
| GABA reuptake inhibitors | CL-996, deramciclane, gabaculine, guvacine, |
| (increase GABA neurotransmission) | nipecotic acid, NNC-711, NNC 05-2090, SKF- |
| | 89976A, SNAP-5114, tiagabine, and hyperforin |
| GABA analogs | gabapentin, butyric acid, valproic acid, valpromide, |
| (increase GABA neurotransmission) | valnoctamide, 3-hydroxybutanal, GHB, sodium, |
| | oxybate, aceburic acid, GBL, GHBAL, GHV, GVL, |
| | GHC, GCL, HOCPCA, UMB68, pregabalin, tolibut, |
| | phaclofen, sacolfen, arecaidine, gaboxadol, |
| | isonipecotic acid, 3-Methyl-GABA, AABA, BABA, |
| | DAVA, GAVA, Glutamic acid, hopantenic acid, |
| | piracetam, and vigabatrin |
| GABA prodrugs | L-Glutamine, N-Isonicotinoyl-GABA, picamilon, |
| (increase GABA neurotransmission) | progabide, tolgabide |
| Acetylcholinesterase inhibitors | carbamates, physostigmine, neostigmine, |
| (increase nicotinic and muscarinic | pyridostigmine, ambenonium, demecarium, |
| neurotransmission) | rivastigmine, phenanthrene derivatives, |
| | galantamine, caffeine, rosmarinic acid, alpha- |
| | pinene, piperidines, donepezil, tacrine, |
| | edrophonium, Huperzine A, ladostigil, ungeremine, |
| | lactucopicrin, dyflos, echothiophate, parathion, and |
| | quasi-irreversible acetylcholinesterase inhibitors |
| Serotonin reuptake inhibitors | alaproclate, cericlamine, citalopram, dapoxetine, |
| (increase serotonin neurotransmission) | escitalopram, femoxetine, fluoxetine, fluvoxamine, |
| | ifoxetine, indalpine, omiloxetine, panuramine, |
| | paroxetine, pirandamine, RTI-353, sertraline, |
| | zimelidine, desmethylcitalopram, |
| | didesmethylcitalopram, seproxetine ((S)- |
| | norfluoxetine), desvenlafaxine, cianopramine, |
| | litoxetine, lubazodone, SB-649,915, trazodone, |
| | vilazodone, vortioxetine, dextromethorphan, |
| | dextropropoxyphene, dimenhydrinate, |
| | diphenhydramine, mepyramine (pyrilamine), |
| | mifepristone, delucemine, mesembrenone, |
| | mesembrine, roxindole, duloxetine, |
| | levomilnacipran, milnacipran, dapoxetine, |
| | sibutramine, chlorpheniramine, |
| | dextropmethorphan, and methadone |
| Serotonin releasing agents | chlorphentermine, cloforex, dexfenfluramine, |
| (increase serotonin neurotransmission) | etolorex, fenfluramine, flucetorex, indeloxazine, |
| | levofenfluramine, tramadol, carbamazepine, |
| | amiflamine (FLA-336), viqualine (PK-5078), 2- |
| | Methyl-3,4-methylenedioxyamphetamine (2-Methyl- |
| | MDA), 3-Methoxy-4-methylamphetamine (MMA), 3- |
| | Methyl-4,5-methylenedioxyamphetamine (5-Methyl- |
| | MDA), 3,4-Ethylenedioxy-N-methylamphetamine |
| | (EDMA), 4-Methoxyamphetamine (PMA), 4- |
| | Methoxy-N-ethylamphetamine (PMEA), 4-Methoxy- |
| | N-methylamphetamine (PMMA), 4- |
| | Methylthioamphetamine (4-MTA), 5-(2- |
| | Aminopropyl)-2,3-dihydrobenzofuran (5-APDB), 5- |
| | Indanyl-2-aminopropane (IAP), 5-Methoxy-6- |
| | methylaminoindane (MMAI), 5-Trifluoromethyl-2- |
| | aminoindane (TAI), 5,6-Methylenedioxy-2- |
| | aminoindane (MDAI), 5,6-Methylenedioxy-N- |
| | methyl-2-aminoindane (MDMAI), 6-Chloro-2- |
| | aminotetralin (6-CAT), 6-Tetralinyl-2-aminopropane |
| | (TAP), 6,7-Methylenedioxy-2-aminotetralin (MDAT), |
| | 6,7-Methylenedioxy-N-methyl-2-aminotetralin |
| | (MDMAT), N-Ethyl-5-trifluoromethyl-2-aminoindane |
| | (ETAI), N-Methyl-5-indanyl-2-aminopropane, |
| | aminorex, MDMA, MDEA, MDA, MBDB, and |
| | tryptamines, such as DMT, αMT, 5MeO-NMT, |
| | NMT, NETP, Dimethyl-Serotonin, 5MeO-NET, αET |
| | and αMT |
| Excitatory amino acid reuptake inhibitors | didydrokanic acid, WAY-213,613, L-trans-2,4-PDC, |
| (increase Glutamate receptor neurotransmission) | amphetamine, and L-Theanine |
| Glycine reuptake inhibitors | bitopertin, Org 24598, Org 25935, ALX-5407, |
| (increase Glutamate receptor neurotransmission) | sacrosine, Org 25543, and N-arachidonylglycerine |
| Histidine decarboxylase inhibitors | Tritoqualine, catechin |
| (decrease histamine neurotransmission) | |
| Endocannabinoid enhancers | AM404, fatty acid amide hydrolase inhibitors (e.g., |
| (increase cannabinoid neurotransmission) | AM374, ARN2508, BIA 10-2472, BMS-469908, |
| | CAY-10402, JNJ-245, JNJ-1661010, JNJ- |
| | 28833155, JNJ-40413269, JNJ-42119779, JNJ- |
| | 42165279, MK-3168, MK-4409, MM-433593, OL- |
| | 92, OL-135, PF-622, PF-750, PF-3845, PF- |
| | 04457845, PF-04862853, RN-450, SA-47, SA-73, |
| | SSR-411298, ST-4068, TK-25, URB524, URB597, |
| | URB694, URB937, VER-156084, and V-158866 |
| Monoacylglycerol lipase inhibitors | N-arachidonoyl maleimide, JZL184 |
| (increase cannabinoid neurotransmission) | |
| Endocannabinoid transporter inhibitors | SB-FI-26 |
| (increase cannabinoid neurotransmission) | |
| Endocannabinoid reuptake inhibitors | AM404, AM1172, LY-2183240, O-2093, OMDM-2, |
| (increase cannabinoid neurotransmission) | UCM-707, VDM-11, guineensine, ETI-T-24_B_I, |
| | WOBE437, and RX-055 |
| Adenosine uptake inhibitors | cilostazol, dilazep, and dipyramidole |
| (increase purinergic neurotransmission) | |
| Nucleoside transporter inhibitors | 8MDP, Decynium 22, 5-iodotubercidin, NBMPR, |
| (increase purinergic neurotransmission) | and TC-T 6000 |

In some embodiments, the neurotransmission modulator is a neurotoxin listed in Table 9, or a functional fragment or variant thereof. Neurotoxins include, without limitation, convulsants, nerve agents, parasympathomimetics, and uranyl compounds. Neurotoxins may be bacterial in origin, or fungal in origin, or plant in origin, or derived from a venom or other natural product. Neurotoxins may be synthetic or engineered molecules, derived de novo or from a natural product. Suitable neurotoxins include but are not limited to botulinum toxin and conotoxin. Exemplary neurotoxins are listed in Table 9.

TABLE 9
NEUROTOXINS
NEUROTOXINS

2,4,5-Trihydroxyamphetamine
2,4,5-Trihydroxymethamphetamine
3,4-Dichloroamphetamine
5,7-Dihydroxytryptamine
5-Iodowillardiine
Ablomin
Aconitine
Aconitum
Aconitum anthora
AETX
Agelenin
Agitoxin
Aldrin
Alpha-Methyldopamine
Alpha-neurotoxin
Altitoxin
Anatoxin-a
Androctonus australis hector insect toxin
Anisatin
Anthopleurin
Antillatoxin
Anuroctoxin
Apamin
Arum italicum
Arum maculatum
Babycurus toxin 1
Batrachotoxin
BDS-1
Bestoxin
Beta-Methylamino-L-alanine
BgK
Birtoxin
BmKAEP
BmTx3
BotlT2
BotlT6
Botulinum toxin
Brevetoxin
Bukatoxin
Butantoxin
Calcicludine
Calciseptine
Calitoxin
Caramboxin
Carbon disulfide
CgNa toxin
Charybdotoxin
Cicutoxin
Ciguatoxin
Cll1
Clostridium botulinum
Conantokins
Conhydrine
Coniine
Conotoxin
Contryphan
Cssll
CSTX
Curare
Cyanide poisoning
Cylindrospermopsin
Cypermethrin
Delta atracotoxin
Dendrotoxin
Dieldrin
Diisopropyl fluorophosphates
Dimethylmercury
Discrepin
Domoic acid
Dortoxin
DSP-4
Ergtoxin
Falcarinol
Fenpropathrin
Gabaculine
Ginkgotoxin
Grammotoxin
Grayanotoxin
Hainantoxin
Halcurin
Hefutoxin
Helothermine
Heteroscodratoxin-1
Histrionicotoxin
Homoquinolinic acid
Hongotoxin
Huwentoxin
Ibotenic acid
Ikitoxin
inhibitor cystine knot
Jingzhaotoxin
Kainic acid
Kaliseptine
Kappa-bungarotoxin
Kodaikanal mercury poisoning
Kurtoxin
Latrotoxin
Lq2
Maitotoxin
Margatoxin
Maurotoxin
Mercury (element)
Methanol
Methiocarb
MPP+
MPTP
Nemertelline
Neosaxitoxin
Nicotine
N-Methylconiine
Oenanthotoxin
Oxalyldiaminopropionic acid
Oxidopamine
Oxotoxin
Pahutoxin
Palytoxin
Pandinotoxin
Para-Bromoamphetamine
Para-Chloroamphetamine
Para-Chloromethamphetamine
Para-Iodoamphetamine
Penitrem A
Phaiodotoxin
Phenol
Phoneutria nigriventer toxin-3
Phrixotoxin
Polyacrylamide
Poneratoxin
Psalmotoxin
Pumiliotoxin
Quinolinic acid
Raventoxin
Resiniferatoxin
Samandarin
Saxitoxin
Scyllatoxin
Sea anemone neurotoxin
Slotoxin
SNX-482
Stichodactyla toxin
Taicatoxin
Taipoxin
Tamapin
Tertiapin
Tetanospasmin
Tetraethylammonium
Tetramethylenedisulfotetramine
Tetrodotoxin
Tityustoxin
Tricresyl phosphate
TsIV
Vanillotoxin
Veratridine

Antibodies

Neurotransmission modulators also include antibodies that bind to neurotransmitters or neurotransmitter receptors listed in Tables 5 and 6 and decrease neurotransmission. These antibodies include blocking and neutralizing antibodies. Antibodies to neurotransmitters or neurotransmitter receptors listed in Tables 5 and 6 can be generated by those of skill in the art using well established and routine methods.

### Neuronal Growth Factor Modulators

In some embodiments, the Activator of nAChRs containing a nAChRα6 subunit is administered with a neuronal growth factor modulator (e.g., an agent that decreases or increases neurogenic/axonogenic signals, e.g., a neuronal growth factor or neuronal growth factor mimic, or an agonist or antagonist of a neuronal growth factor or neuronal growth factor receptor). For example, the neuronal growth factor modulator is a neuronal growth factor listed in Table 10, e.g., a neuronal growth factor having the sequence referenced by accession number or Entrez Gene ID in Table 10, or an analog thereof, e.g., a sequence having at least 75%, 80%, 85%, 90%, 90%, 98%, 99% identity to the sequence referenced by accession number or Entrez Gene ID in Table 10. Neuronal growth factor modulators also include agonists and antagonists of neuronal growth factors and neuronal growth factor receptors listed in Table 10. A neuronal growth factor modulator may increase or decrease neurogenesis, neuronal growth, neuronal differentiation, neurite outgrowth, synapse formation, synaptic maturation, synaptic refinement, or synaptic stabilization. Neuronal growth factor modulators regulate tissue innervation (e.g., innervation of a lymph node) and the formation of synaptic connections between two or more neurons and between neurons and non-neural cells (e.g., between neurons and immune cells). A neuronal growth factor modulator may block one or more of these processes (e.g., through the use of antibodies that block neuronal growth factors or their receptors) or promote one or more of these processes (e.g., through the use of neuronal growth factors or analogs thereof). Neuronal growth factor modulators can increase or decrease one of the above mentioned processes by 10%, 20%, 30%, 40%, 50%, 60%, 70%, 80%, 90%, 95%, 98%, 200%, 500% or more.

In some embodiments, the neuronal growth factor modulator is one that increases neurogenic/axonogenic signals, e.g., the method includes administering to the subject or contacting a cell with a neuronal growth factor modulator in an amount and for a time sufficient to increase neurogenesis or axonogenesis. For example, the neuronal growth factor modulator that leads to an increase in neurogenesis or axonogenesis is a neurotrophic factor. Relevant neurotrophic factors include NGF, BDNF, ProNGF, Sortilin, TGFβ and TGFβ family ligands and receptors (e.g., TGFβR1, TGFβR2, TGFβ1, TGFβ2 TGFβ4), GFRa family ligands and receptors (e.g., GFRα1, GFRα2, GFRα3, GFRα4, GDNF), CNTF, LIF, neurturin, artemin, persephin, neurotrophin, chemokines, cytokines, and others listed in Table 10. Receptors for these factors may also be targeted, as well as downstream signaling pathways including Jak-Stat inducers, and cell cycle and MAPK signaling pathways. In some embodiments, the neuronal growth factor modulator increases neurogenesis, axonogenesis or any of the processes mentioned above by administering, locally delivering, or stabilizing a neuronal growth factor listed in Table 10, or by upregulating, agonizing, or stabilizing a neuronal growth factor receptor listed in Table 10. In some embodiments, the neuronal growth factor modulator increases neurogenesis, axonogenesis or any of the processes mentioned above by stabilizing, agonizing, overexpressing, or upregulating a signaling protein encoded by a gene that is downstream of a neuronal growth factor. In some embodiments, the neuronal growth factor modulator increases neurogenesis, axonogenesis or any of the processes mentioned above by stabilizing, overexpressing, or upregulating a synaptic or structural protein. Neurogenesis, axonogenesis, neuronal growth, neuronal differentiation, neurite outgrowth, synapse formation, synaptic maturation, synaptic refinement, or synaptic stabilization can be increased in the subject at least 1%, 2%, 5%, 10%, 15%, 20%, 25%, 30%, 35%, 40%, 50%, 60%, 70%, 80% or more, compared to before the administration. Neurogenesis, axonogenesis, neuronal growth, neuronal differentiation, neurite outgrowth, synapse formation, synaptic maturation, synaptic refinement, or synaptic stabilization can be increased in the subject between 5-20%, between 5-50%, between 10-50%, between 20-80%, between 20-70%.

In some embodiments, the neuronal growth factor modulator decreases neurogenic/axonogenic signals, e.g., the method includes administering to the subject or contacting a cell with a neuronal growth factor modulator in an amount and for a time sufficient to decrease neurogenesis, axonogenesis, or innervation. For example, the neuronal growth factor modulator that leads to a decrease in neurogenesis or axonogenesis is a blocking or neutralizing antibody against a neurotrophic factor. Relevant neurotrophic factors include NGF, BDNF, ProNGF, Sortilin, TGFβ and TGFβ family ligands and receptors (e.g., TGFβR1, TGFβR2, TGFβ1, TGFβ2 TGFβ4), GFRa family ligands and receptors (e.g., GFRα1, GFRα2, GFRα3, GFRα4, GDNF), CNTF, LIF, neurturin, artemin, persephin, neurotrophin, chemokines, cytokines, and others listed in Table 10. Receptors for these factors can also be targeted, as well as downstream signaling pathways including Jak-Stat inducers, and cell cycle and MAPK signaling pathways. In some embodiments, the neuronal growth factor modulator decreases neurogenesis, axonogenesis or any of the processes mentioned above by sequestering, blocking, antagonizing, degrading, or downregulating a neuronal growth factor or a neuronal growth factor receptor listed in Table 10. In some embodiments, the neuronal growth factor modulator decreases neurogenesis, axonogenesis or any of the processes mentioned above by blocking or antagonizing a signaling protein that is downstream of a neuronal growth factor. In some embodiments, the neuronal growth factor modulator decreases neurogenesis, axonogenesis or any of the processes mentioned above by blocking, disrupting, or antagonizing a synaptic or structural protein. Neurogenesis, axonogenesis, neuronal growth, neuronal differentiation, neurite outgrowth, synapse formation, synaptic maturation, synaptic refinement, synaptic stabilization, or tissue innervation can be decreased in the subject at least 1%, 2%, 5%, 10%, 15%, 20%, 25%, 30%, 35%, 40%, 50%, 60%, 70%, 80% or more, compared to before the administration. Neurogenesis, axonogenesis, neuronal growth, neuronal differentiation, neurite outgrowth, synapse formation, synaptic maturation, synaptic refinement, synaptic stabilization, or tissue innervation can be decreased in the subject between 5-20%, between 5-50%, between 10-50%, between 20-80%, between 20-70%. Neuronal growth factor blockers can be administered in any of the modalities described herein (e.g., antibody, small molecule, nucleic acid, polypeptide, or viral vector).

In some embodiments, the neuronal growth factor modulator increases or decreases the number of nerves in an affected tissue (e.g., a lymph node or secondary or tertiary lymphoid organ). For example, the neuronal growth factor modulator is administered in an amount and for a time sufficient to increase or decrease neurogenesis/axonogenesis.

Neuronal growth factor blockers include antibodies that bind to neuronal growth factors or neuronal growth factor receptors and decrease their signaling (e.g., blocking antibodies). Exemplary neuronal growth factor blocking antibodies are listed below in Table 10. Antibodies to neuronal growth factors listed in Table 11 can also be generated by those of skill in the art using well established and routine methods.

TABLE 10
NEURONAL GROWTH FACTORS

| | | Accession | Entrez |
|---|---|---|---|
| Gene | Type | Number | Gene ID |
| ARTN | Ligand | Q5T4W7 | 9048 |
| BDNF | Ligand | P23560 | 627 |
| BDNF-AS | Ligand | | 497258 |
| BEX1 | Signaling | Q9HBH7 | 55859 |
| BEX3 | Signaling | Q00994 | 27018 |
| CD34 | Receptor | P28906 | 947 |
| CDNF | Ligand | Q49AH0 | 441549 |
| CNTF | Ligand | P26441 | 1270 |
| CNTFR | Receptor | P26992 | 1271 |
| CRLF1 | Receptor | O75462 | 9244 |
| CSPG5 | Ligand | O95196 | 10675 |
| DCLK1 | Signaling | O15075 | 9201 |
| DISC1 | Signaling | Q9NRI5 | 27185 |
| DNAJC5 | Signaling | Q9H3Z4 | 80331 |
| DPYSL2 | Signaling | Q16555 | 1808 |
| DVL1 | Signaling | O14640 | 1855 |
| EFNA5 | Ligand | P52803 | 1946 |
| EGR3 | Signaling | Q06889 | 1960 |
| ENO2 | Signaling | P09104 | 2026 |
| EphA1 | Receptor | P21709 | 2041 |
| EphA10 | Receptor | Q5JZY3 | 284656 |
| EphA2 | Receptor | P29317 | 1969 |
| EphA3 | Receptor | P29320 | 2042 |
| EphA4 | Receptor | P29317 | 2043 |
| EphA5 | Receptor | P54756 | 2044 |
| EphA6 | Receptor | Q9UF33 | 285220 |
| EphA7 | Receptor | Q15375 | 2045 |
| EphA8 | Receptor | P29322 | 2046 |
| EphB1 | Receptor | P54762 | 2047 |
| EphB2 | Receptor | P29323 | 2048 |
| EphB3 | Receptor | P54753 | 2049 |
| EphB4 | Receptor | P54760 | 2050 |
| EphB6 | Receptor | O15197 | 2051 |
| ETBR2 | Receptor | O60883 | 9283 |
| FSTL4 | Receptor | Q6MZW2 | 23105 |
| GDNF | Ligand | P39905 | 2668 |
| GFRA1 | Receptor | P56159 | 2674 |
| GFRA2 | Receptor | O00451 | 2675 |
| GFRA3 | Receptor | O60609 | 2676 |
| GFRA4 | Receptor | Q9GZZ7 | 64096 |
| GPR37 | Receptor | O15354 | 2861 |
| GPRIN1 | Signaling | Q7Z2K8 | 114787 |
| GPRIN2 | Signaling | O60269 | 9721 |
| GPRIN3 | Signaling | Q6ZVF9 | 285513 |
| GRB2 | Signaling | P62993 | 2885 |
| GZF1 | Signaling | Q9H116 | 64412 |
| IFNA1 | Ligand | P01562 | 3439 |
| IGF1 | Ligand | P05019 | 3479 |
| IGF2 | Ligand | P01344 | 3481 |
| IL11RA | Receptor | Q14626 | 3590 |
| IL1B | Ligand | P01584 | 3553 |
| IL3 | Ligand | P08700 | 3562 |
| IL4 | Ligand | P05112 | 3565 |
| IL6 | Ligand | P05231 | 3569 |
| IL6R | Receptor | P08887 | 3570 |
| IL6ST | Signaling | P40189 | 3572 |
| INS | Ligand | P01308 | 3630 |
| L1CAM | Signaling | P32004 | 3897 |
| LIF | Ligand | P15018 | 3976 |
| LIFR | Receptor | P42702 | 3977 |
| MAGED1 | Signaling | Q9Y5V3 | 9500 |
| MANF | Ligand | P55145 | 7873 |
| NDNF | Ligand | Q8TB73 | 79625 |
| NENF | Ligand | Q9UMX5 | 29937 |
| NENFP1 | Ligand | | 106480294 |
| NENFP2 | Ligand | | 100129880 |
| NENFP3 | Ligand | | 106481703 |
| NGF | Ligand | P01138 | 4803 |
| NGFR | Receptor | P08138 | 4804 |
| NRG1 | Ligand | Q02297 | 3084 |
| NRP1 | Receptor | O14786 | 8829 |
| NRTN | Ligand | Q99748 | 902 |
| NTF3 | Ligand | P20783 | 4908 |
| NTF4 | Ligand | P34130 | 4909 |
| NTRK1 | Receptor | P04629 | 4914 |
| NTRK2 | Receptor | Q16620 | 4915 |
| NTRK3 | Receptor | Q16288 | 4916 |
| PDPK1 | Signaling | O15530 | 5170 |
| PEDF | Ligand | P36955 | 5176 |
| PLEKHH3 | Signaling | Q7Z736 | 79990 |
| PSAP | Ligand | P07602 | 5660 |
| PSEN1 | Signaling | P49768 | 5663 |
| PSPN | Ligand | O70300 | 5623 |
| PTN | Ligand | P21246 | 5764 |
| RELN | Ligand | P78509 | 5649 |
| RET | Signaling | P07949 | 5979 |
| ROR1 | Receptor | Q01973 | 4919 |
| ROR2 | Receptor | Q01974 | 4920 |
| RPS6KA3 | Signaling | P51812 | 6197 |
| SDC3 | Receptor | O75056 | 9672 |
| SEMA3E | Ligand | O15041 | 9723 |
| SERPINE2 | Ligand | P07093 | 5270 |
| SERPINF1 | Ligand | P36955 | 5176 |
| SHC1 | Signaling | P51812 | 6464 |
| SNTG1 | Biosynthesis | P07602 | 54212 |
| SORCS1 | Receptor | O75056 | 114815 |
| SORCS2 | Receptor | O15041 | 57537 |
| SORCS3 | Receptor | P07093 | 22986 |
| SORT1 | Receptor | Q99523 | 6272 |
| SULF1 | Signaling | Q8IWU6 | 23213 |
| SULF2 | Signaling | Q8IWU5 | 55959 |
| TGFB1 | Ligand | P01137 | 7040 |
| TGFB2 | Ligand | P61812 | 7042 |
| TGFB3 | Ligand | P10600 | 7043 |
| TMEM158 | Receptor | Q8WZ71 | 25907 |
| TNF | Ligand | P01375 | 7124 |
| TPM3 | Receptor | P06753 | 7170 |
| VEGFA | Ligand | P15692 | 7422 |
| VEGFB | Ligand | P49765 | 7423 |
| VGF | Ligand | O15240 | 7425 |
| XCR1 | Receptor | P46094 | 2829 |
| ZN274 | Signaling | Q96GC6 | 10782 |

TABLE 11
NEURONAL GROWTH FACTOR ANTIBODIES

| Neuronal | | |
|---|---|---|
| Growth | | |
| Factor | Antibody | Company |
| BDNF | 38B8 (agonist antibody) | Pfizer |
| BDNF | 29D7 (agonist antibody) | Pfizer |
| EphA3 | KB004 | KaloBios Pharmaceuticals, Inc. |
| IFNA1 | Faralimomab | Creative Biolabs |
| IFNA1 | Sifalimumab (MEDI-545) | MedImmune |
| IFNA1 | Rontalizumab | Genentech |
| IGF | Figitumumab (CP-751,871) - an | Pfizer |
| | IGR-1R MAb | |
| IGF | SCH717454 (Robatumamab, | Merck |
| | inhibits IGF initiated | |
| | phosphorylation) | |
| IGF | Cixutumumab (IGF-1R antibody) | Eli Lilly |
| IGF | Teprotumumab (IGF-1R | Genmab/Roche |
| | blocking antibody) | |
| IGF-2 | Dusigitumab | MedImmune/AstraZeneca |
| IGF-2 | DX-2647 | Dyax/Shire |
| IGF | Xentuzumab | Boehringer Ingelheim/Eli Lilly |
| IGF | Dalotuzumab (IGFR1 blocking | Merck & Co. |
| | antibody) | |
| IGF | Figitumumab (IGFR1 blocking | Pfizer |
| | antibody) | |
| IGF | Ganitumab (IGFR1 blocking | Amgen |
| | antibody) | |
| IGF | Robatumumab (IGFR1 blocking | Roche/Schering-Plough |
| | antibody) | |
| IL1B | Canakinumab | Novartis |
| IL1B | APX002 | Apexigen |
| IL1B | Gevokizumab | XOMA |
| IL4 | Pascolizumab | GlaxoSmithKline |
| IL4 | Dupilumab | Regeneraon/Sanofi |
| IL6 | Siltuximab | Janssen Biotech, Inc. |
| IL6 | Olokizumab | UCB/R-Pharm |
| IL6 | Elsilimomab | Orphan Pharma International |
| IL6 | Sirukumab | Centocor |
| IL6 | Clazakizumab | Bristol Myers Squib/Alder |
| | | Biopharmaceuticals |
| IL6 | Gerilimzumab (ARGX-109) | arGEN-X/RuiYi |
| IL6 | FE301 | Ferring Pharmaceuticals |
| IL6 | FM101 | Femta Pharmaceuticals |
| IL-6R | Sarilumab (directed against | Regeneron/Sanofi |
| | IL6R) | |
| IL-6R | Tocilizumab | Hoffmann-La Roche/Chugai |
| IL-6R | Sapelizumab | Chugai |
| IL-6R | Vobarilizumab | Ablynx |
| L1CAM | AB417 | Creative biolabs |
| L1CAM | L1-9.3 | Creative biolabs |
| L1CAM | L1-14.10 | Biolegend |
| NGF | Tanezumab | Pfizer |
| NGF | Fulranumab (JNJ-42160443), | Amgen |
| NGF | MNAC13 (anti-TrkA, the NGF | Creative Biolabs |
| | receptor) | |
| NGF | mAb 911 | Rinat/Pfizer |
| NGF | Fasinumab | Regeneron/Teva |
| NRG1 | 538.24 | Hoffman-La Roche |
| NRP1 | Vesencumab | Genentech/Roche |
| ROR1 | Cirmtuzumab | Oncternal Therapeutics |
| SAP | GSK2398852 | GlaxoSmithKline |
| TGFβ | Fresolimumab (pan-TGFβ | Genzyme/Aventis |
| | antibody) | |
| TGFβ | IMC-TR1 (LY3022859) (MAb | Eli Lilly |
| | against TGFβRII) | |
| TGFβ | TβM1 (anti-TGFβ1 MAb) | Eli Lilly |
| TGFβ2 | Lerdelimumab (CAT-152) | Genzyme |
| TGFβ1 | Metelimumab | Genzyme |
| TGFβ1 | LY2382770 | Eli Lilly |
| TGFβ | PF-03446962 (MAb against | Pfizer |
| | TGFβRI) | |
| TNF | Infliximab | Janssen Biotech, Inc. |
| TNF | Adalimumab | AbbVie Inc. |
| TNF | Certolizumab pegol | UCB |
| TNF | Golimumab | Janssen Biotech, Inc. |
| TNF | Afelimomab | |
| TNF | Placulumab | Teva Pharmaceutical Industries, |
| | | Inc. |
| TNF | Nerelimomab | Chiron/Celltech |
| TNF | Ozoralizumab | Pfizer/Ablynx |
| VEGFA | Bevacizumab | Genentech |
| VEGFA | Ranibizumab | Genentech |
| VEGF | Alacizumab pegol (anti- | UCB |
| | VEGFR2) | |
| VEGFA | Brolucizumab | Novartis |
| VEGF | Icrucumab (anti-VEGFR1) | Eli Lilly |
| VEGF | Ramucirumab (anti-VEGFR2) | Eli Lilly |

Neuronal growth factor modulators also include agents that agonize or antagonize neuronal growth factors and neuronal growth factor receptors. For example, neuronal growth factor modulators include TNF inhibitors (e.g., etanercept, thalidomide, lenalidomide, pomalidomide, pentoxifylline, bupropion, and DOI), TGFβ1 inhibitors, (e.g., disitertide (P144)), TGFβ2 inhibitors (e.g., trabedersen (AP12009)). Exemplary neuronal growth factor agonists and antagonists are listed in Table 12.

TABLE 12
NEURONAL GROWTH FACTOR AGONISTS AND ANTAGONISTS

| | Agonist | Antagonist |
|---|---|---|
| TrkA | NGF, amitriptyline, and | ALE-0540 |
| | gambogic amide, gambogic acid | |
| TrkB | BDNF, NT3, NT4, 3,7- | ANA-12, cyclotraxin B, and |
| | Dihydroxyflavone, 3,7,8,2′- | gossypetin |
| | Tetrahydroxyflavone, 4′- | |
| | Dimethylamino-7,8- | |
| | dihydroxyflavone, 7,3′- | |
| | Dihydroxyflavone, 7,8- | |
| | Dihydroxyflavone, 7,8,2′- | |
| | Trihydroxyflavone, 7,8,3′- | |
| | Trihydroxyflavone, Amitriptyline, | |
| | Deoxygedunin, Diosmetin, | |
| | HIOC, LM22A-4, N- | |
| | Acetylserotonin, Norwogonin | |
| | (5,7,8-THF), R7, LM22A4, and | |
| | TDP6 | |
| Pan-Trk receptor | | entrectinib (RXDX-101), AG |
| | | 879, GNF 5837, GW 441756, |
| | | and PF 06273340 |
| GFRα1R | GDNF and XIB4035 | |
| VEGF receptor | | AEE 788, AG 879, AP 24534, |
| | | axitinib, DMH4, GSK 1363089, |
| | | Ki 8751, RAF 265, SU 4312, SU |
| | | 5402, SU 5416, SU 6668, |
| | | sunitinib, toceranib, vatalanib, |
| | | XL 184, ZM 306416, and ZM |
| | | 323881 |
| TGFβRI | | galunisertib (LY2157299), TEW- |
| | | 7197, SB-431542, A 83-01, D |
| | | 4476, GW 788388, LY 364947, |
| | | R 268712, RepSox, SB 505124, |
| | | SB 525334, and SD 208 |

In any of the combination therapy approaches described herein, the first and second therapeutic agent (e.g., an α6*nAChR activator described herein and the additional therapeutic agent) are administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours or up to 1-7, 1-14, 1-21 or 1-30 days before or after the second therapeutic agent.

Diagnosis and Prognosis of α6*nAChR-Associated Inflammatory or Autoimmune Diseases or Conditions

The methods described herein include methods of diagnosing or identifying patients with an α6*nAChR-associated inflammatory or autoimmune disease or condition. Subjects who can be diagnosed or identified as having an α6*nAChR-associated inflammatory or autoimmune disease or condition are subjects who have an inflammatory or autoimmune disease or condition (e.g., subjects identified as having an inflammatory or autoimmune disease or condition), or subjects suspected of having an inflammatory or autoimmune disease or condition. Subjects can be diagnosed or identified as having an α6*nAChR-associated inflammatory or autoimmune disease or condition based on screening of patient samples (e.g., immune cells collected from a subject, e.g., Tregs). nAChRα6 expression (e.g., CHRNA6 gene or nAChRα6 subunit protein expression) can be assessed in a sample of immune cells isolated from a subject using standard techniques known in the art, such as immunohistochemistry, western blot analysis, quantitative RT-PCR, RNA sequencing, fluorescent in situ hybridization, cDNA microarray, and droplet digital PCR. nAChRα6 expression can be assessed by comparing measurements obtained from immune cells collected from a subject having or suspected of having an inflammatory or autoimmune disease or condition to measurements of nAChRα6 expression obtained from a reference sample (e.g., immune cells of the same type collected from a subject that does not have an inflammatory or autoimmune disease or condition or a cell that does not express nAChRα6, e.g., a HEK cell). Reference samples can be obtained from healthy subjects (e.g., subjects without an inflammatory or autoimmune disease or condition), or they can be obtained from databases in which average measurements of nAChRα6 expression are cataloged for immune cells from healthy subjects (e.g., subjects without an inflammatory or autoimmune disease or condition).

Subjects are diagnosed or identified as having an α6*nAChR-associated inflammatory or autoimmune disease or condition if nAChRα6 expression (e.g., CHRNA6 gene or nAChRα6 subunit protein expression) is decreased in the sample of immune cells from the subject compared to the reference sample. A decrease of nAChRα6 expression of 1.1-fold or more (e.g., 1.1, 1.2, 1.3, 1.4, 1.5, 2.0, 2.5, 3.0, 3.5, 4.0, 4.5, 5.0, 6.0, 7.0, 8.0, 9.0, 10.0-fold or more) in the immune cell sample compared to the reference indicates that the subject has α6*nAChR-associated inflammatory or autoimmune disease or condition. Subjects diagnosed or identified as having α6*nAChR-associated inflammatory or autoimmune disease or condition can be treated with the methods and compositions described herein (e.g., α6*nAChR activators α6*nAChR activators). Subjects with an autoimmune or inflammatory disease or condition can also be treated with the methods and compositions described herein if an immune cell from the subject (e.g., a Treg) is found to express α6*nAChR (e.g., CHRNA6 gene or nAChRα6 subunit protein expression).

The methods described herein also include methods of predicting patient response (e.g., the response of an inflammatory or autoimmune disease or condition in a subject) to α6*nAChR activators α6*nAChR activators in order to determine α6*nAChR activators α6*nAChR activators can be used for treatment of an inflammatory or autoimmune disease or condition. In some embodiments, a sample (e.g., an immune cell or tissue sample) is isolated from a subject and contacted with one or more α6*nAChR activators α6*nAChR activators (e.g., samples are cultured and contacted with one or more activators in vitro). The response of the sample (e.g., immune cell or tissue sample, e.g., a Treg) to the one or more α6*nAChR activators α6*nAChR activators is evaluated to predict response to treatment. Responses that are evaluated include immune cell migration, proliferation, recruitment, lymph node homing, lymph node egress, differentiation, activation, polarization, cytokine production, degranulation, maturation, ADCC, ADCP, antigen presentation, and/or immune cell nAChRα6 expression. A decrease of at least 5% or more (e.g., 5%, 10%, 15%, 20%, 25%, 30%, 40%, 50%, 60%, 70%, 80%, 90%, 99%, or more) in markers of inflammation in treated cells compared to untreated or control-treated cells, or an increase of at least 5% or more (e.g., 5%, 10%, 15%, 20%, 25%, 30%, 40%, 50%, 60%, 70%, 80%, 90%, 99%, or more) in migration, proliferation, recruitment, activation, anti-inflammatory cytokine production (e.g., production of IL-10 and/or TGFβ), or nAChRα6 expression in treated cells (e.g., Tregs) compared to untreated or control-treated cells indicates that the inflammatory or autoimmune disease or condition would respond to treatment with an α6*nAChR activator.

The methods used above to diagnose or identify a subject with α6*nAChR-associated inflammatory or autoimmune disease or condition can also be used to predict patient response (e.g., the response of an inflammatory or autoimmune disease or condition in a subject) to treatment with an α6*nAChR activator. If the expression of α6*nAChR (e.g., CHRNA6 gene or nAChRα6 subunit protein expression) is decreased in an immune cell sample compared to a reference (e.g., 1.1, 1.2, 1.3, 1.4, 1.5, 2.0, 2.5, 3.0, 3.5, 4.0, 4.5, 5.0, 6.0, 7.0, 8.0, 9.0, 10.0-fold or more), the subject can be predicted to respond to treatment with an α6*nAChR activator. Subjects predicted to respond to treatment with an α6*nAChR activator or α6*nAChR-specific activator can be treated using the methods and compositions described herein (e.g., α6*nAChR activators).

### Methods of Treatment

Administration

An effective amount of an α6*nAChR activator α6*nAChR activator described herein for treatment of an inflammatory or autoimmune disease or condition can be administered to a subject by standard methods. For example, the agent can be administered by any of a number of different routes including, e.g., intravenous, intradermal, subcutaneous, percutaneous injection, oral, transdermal (topical), or transmucosal. The α6*nAChR activator α6*nAChR activator can be administered orally or administered by injection, e.g., intramuscularly, or intravenously. The most suitable route for administration in any given case will depend on the particular agent administered, the patient, the particular disease or condition being treated, pharmaceutical formulation methods, administration methods (e.g., administration time and administration route), the patients age, body weight, sex, severity of the diseases being treated, the patient's diet, and the patient's excretion rate. The agent can be encapsulated or injected, e.g., in a viscous form, for delivery to a chosen site, e.g., a site of inflammation. The agent can be provided in a matrix capable of delivering the agent to the chosen site. Matrices can provide slow release of the agent and provide proper presentation and appropriate environment for cellular infiltration. Matrices can be formed of materials presently in use for other implanted medical applications. The choice of matrix material is based on any one or more of: biocompatibility, biodegradability, mechanical properties, and cosmetic appearance and interface properties. One example is a collagen matrix.

The agent (e.g., α6*nAChR activator α6*nAChR activator, e.g., small molecule or antibody) can be incorporated into pharmaceutical compositions suitable for administration to a subject, e.g., a human. Such compositions typically include the agent and a pharmaceutically acceptable carrier. As used herein the term “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances are known. Except insofar as any conventional media or agent is incompatible with the active compound, such media can be used in the compositions of the invention. Supplementary active compounds can also be incorporated into the compositions.

A pharmaceutical composition can be formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an α6*nAChR activator α6*nAChR activator described herein) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions (including liposomes targeted to infected cells with monoclonal antibodies to viral antigens) can also be used as pharmaceutically acceptable carriers. These can be prepared according to methods known to those skilled in the art.

Nucleic acid molecule agents described herein can be administered directly (e.g., therapeutic mRNAs) or inserted into vectors used as gene therapy vectors. Gene therapy vectors can be delivered to a subject by, for example, intravenous injection, local administration (see U.S. Pat. No. 5,328,470) or by stereotactic injection (see, e.g., Chen et al., PNAS 91:3054 1994). The pharmaceutical preparation of the gene therapy vector can include the gene therapy vector in an acceptable diluent, or can include a slow release matrix in which the gene delivery vehicle is embedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

Methods of formulating pharmaceutical agents are known in the art, e.g., Niazi, Handbook of Pharmaceutical Manufacturing Formulations (Second Edition), CRC Press 2009, describes formulation development for liquid, sterile, compressed, semi-compressed and OTC forms. Transdermal and mucosal delivery, lymphatic system delivery, nanoparticles, controlled drug release systems, theranostics, protein and peptide drugs, and biologics delivery are described in Wang et al., Drug Delivery: Principles and Applications (Second Edition), Wiley 2016; formulation and delivery of peptide and protein agent is described, e.g., in Banga, Therapeutic Peptides and Proteins: Formulation, Processing, and Delivery Systems (Third Edition), CRC Press 2015.

Local Administration

The α6*nAChR activators described herein can be administered locally, e.g., to the site associated with the inflammatory or autoimmune disease or condition in the subject. Examples of local administration include epicutaneous, inhalational, intra-articular, intrathecal, intravaginal, intravitreal, intrauterine, intra-lesional administration, lymph node administration, intratumoral administration and administration to a mucous membrane of the subject, wherein the administration is intended to have a local and not a systemic effect. As an example, for the treatment of an inflammatory or autoimmune disease or condition described herein, the α6*nAChR activator α6*nAChR activator may be administered locally (e.g., to or near a lymph node, or to or near a site of inflammation) in a compound-impregnated substrate such as a wafer, microcassette, or resorbable sponge placed in direct contact with the affected tissue. Alternatively, the α6*nAChR activator is infused into the brain or cerebrospinal fluid using standard methods. As yet another example, a pulmonary inflammatory or autoimmune disease or condition described herein (e.g., asthma) may be treated, for example, by administering the α6*nAChR activator locally by inhalation, e.g., in the form of an aerosol spray from a pressured container or dispenser which contains a suitable propellant, e.g., a gas such as carbon dioxide or a nebulizer. An α6*nAChR activator for use in the methods described herein can be administered to a lymph node, spleen, secondary lymphoid organ, tertiary lymphoid organ, barrier tissue, skin, gut, or airway. In certain embodiments, the agent is administered to a mucous membrane of the subject.

Combination Therapy

The α6*nAChR activators described herein may be administered in combination with one or more additional therapies (e.g., 1, 2, 3 or more additional therapeutic agents). The two or more agents can be administered at the same time (e.g., administration of all agents occurs within 15 minutes, 10 minutes, 5 minutes, 2 minutes or less). The agents can also be administered simultaneously via co-formulation. The two or more agents can also be administered sequentially, such that the action of the two or more agents overlaps and their combined effect is such that the reduction in a symptom, or other parameter related to the disorder is greater than what would be observed with one agent or treatment delivered alone or in the absence of the other. The effect of the two or more treatments can be partially additive, wholly additive, or greater than additive (e.g., synergistic). Sequential or substantially simultaneous administration of each therapeutic agent can be effected by any appropriate route including, but not limited to, oral routes, intravenous routes, intramuscular routes, local routes, and direct absorption through mucous membrane tissues. The therapeutic agents can be administered by the same route or by different routes. For example, a first therapeutic agent of the combination may be administered by intravenous injection while a second therapeutic agent of the combination can be administered locally in a compound-impregnated microcassette. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours or up to 1-7, 1-14, 1-21 or 1-30 days before or after the second therapeutic agent.

For use in treating inflammatory and autoimmune related diseases or conditions, the second agent may be a disease-modifying anti-rheumatic drug (DMARD), a biologic response modifier (a type of DMARD), a corticosteroid, a nonsteroidal anti-inflammatory medication (NSAID). In some embodiments, the second agent is prednisone, prednisolone, methylprednisolone, methotrexate, hydroxycholorquine, sulfasalazine, leflunomide, cyclophosphamide, azathioprine, or a biologic such as tofacitinib, adalimumab, abatacept, anakinra, kineret, certolizumab, etanercept, golimumab, infliximab, rituximab or tocilizumab. For example, if the disease is RA, the second agent may be one or more of: prednisone, prednisolone and methylprednisolone, methotrexate, hydroxycholorquine, sulfasalazine, leflunomide, cyclophosphamide and azathioprine, tofacitinib, adalimumab, abatacept, anakinra, kineret, certolizumab, etanercept, golimumab, infliximab, rituximab or tocilizumab. In some embodiments, the second agent is 6-mercaptopurine, 6-thioguanine, abatacept, adalimumab, alemtuzumab (Lemtrada), aminosalicylates (5-aminoalicylic acid, sulfasalazine, mesalamine, balsalazide, olsalazine), antibiotics, anti-histamines, Anti-TNFα (infliximab, adalimumab, certolizumab pegol, natalizumab), azathioprine, belimumab, beta interferon, calcineurin inhibitors, certolizumab, corticosteroids (prednisone, methylprednisolone), cromolyn, cyclosporin A, cyclosporine, dimethyl fumarate (tecfidera), etanercept, fingolimod (Gilenya), fumaric acid esters, glatiramer acetate (Copaxone), golimumab, hydroxyurea, IFNγ, IL-11, infliximab, leflunomide, leukotriene receptor antagonist, long-acting beta2 agonist, methotrexate, mitoxantrone, mycophenolate mofetil, natalizumab (tysabri), NSAIDs, ocrelizumab, pimecrolimus, probiotics (VSL#3), retinoids, rituximab, salicylic acid, short-acting beta2 agonist, sulfasalazine, tacrolimus, teriflunomide (Aubagio), theophylline, tocilizumab, ustekinumab (anti-IL12/IL23), and vedolizumab (Anti alpha3 beta7 integrin).

Dosing

Subjects that can be treated as described herein are subjects with an inflammatory or autoimmune disease or condition. The methods described herein may include a step of selecting a treatment for a patient. The method includes (a) identifying (e.g., diagnosing) a patient who has an autoimmune or inflammatory disease or condition, and (b) selecting an α6*nAChR activator, e.g., an α6*nAChR activator described herein, to treat the condition in the patient. In some embodiments, the method includes administering the selected treatment (e.g., an effective amount of an α6*nAChR activator) to the subject. In some embodiments, the subject has had denervation (e.g., surgical denervation or traumatic denervation such as from spinal cord injury).

In some embodiments, the method includes administering the selected treatment to the subject.

In some embodiments, the agent is administered in an amount and for a time effective to result in one of (or more, e.g., 2 or more, 3 or more, 4 or more of): (a) reduced auto-antibody levels, (b) reduced inflammation, (c) increased organ function (d) reduced pain, (e) decreased rate or number of relapses or flare-ups of the disease, (f) increased quality of life.

The methods described herein can include profiling an immune cell to determine whether it expresses α6*nAChR. Profiling can be performed using RNA sequencing, microarray analysis, or serial analysis of gene expression (SAGE). Other techniques that can be used to assess nAChRα6 expression include quantitative RT-PCR. Profiling results can be confirmed using other methods such as immunohistochemistry, western blot analysis, flow cytometry, or southern blot analysis. Profiling results can be used to determine which α6*nAChR activator should be administered to treat the patient.

An α6*nAChR activator administered according to the methods described herein does not have a direct effect on the central nervous system (CNS) or gut. Any effect on the CNS or gut is reduced compared to the effect observed if the α6*nAChR activator is administered directly to the CNS or gut. In some embodiments, direct effects on the CNS or gut are avoided by modifying the α6*nAChR activator not to cross the BBB, as described herein above, or administering the agent locally to a subject.

Subjects with an inflammatory or autoimmune disease or condition are treated with an effective amount of an α6*nAChR activator. The methods described herein also include contacting immune cells with an effective amount of an α6*nAChR activator. In some embodiments, an effective amount of an α6*nAChR activator is an amount sufficient to increase or decrease lymph node innervation, nerve firing in a lymph node, the development of HEVs or TLOs, immune cell migration, proliferation, recruitment, lymph node homing, lymph node egress, differentiation, activation, polarization, cytokine production, degranulation, maturation, ADCC, ADCP, or antigen presentation. In some embodiments, an effective amount of an α6*nAChR activator is an amount sufficient to treat the autoimmune or inflammatory condition, reduce symptoms of an autoimmune or inflammatory condition, reduce inflammation, reduce auto-antibody levels, increase organ function, or decrease rate or number of relapses or flare-ups.

The methods described herein may also include a step of assessing the subject for a parameter of immune response, e.g., assessing the subject for one or more (e.g., 2 or more, 3 or more, 4 or more) of: Th2 cells, T cells, circulating monocytes, neutrophils, peripheral blood hematopoietic stem cells, macrophages, mast cell degranulation, activated B cells, NKT cells, macrophage phagocytosis, macrophage polarization, antigen presentation, immune cell activation, immune cell proliferation, immune cell lymph node homing or egress, T cell differentiation, immune cell recruitment, immune cell migration, lymph node innervation, dendritic cell maturation, HEV development, TLO development, or cytokine production. In embodiments, the method includes measuring a cytokine or marker associated with the particular immune cell type, as listed in Table 2 (e.g., performing an assay listed in Table 2 for the cytokine or marker). In some embodiments, the method includes measuring a chemokine, receptor, or immune cell trafficking molecule, as listed in Tables 3 and 4 (e.g., performing an assay to measure the chemokine, marker, or receptor). The assessing may be performed after the administration, before the first administration and/or during a course a treatment, e.g., after a first, second, third, fourth or later administration, or periodically over a course of treatment, e.g., once a month, or once every 3 months. In one embodiment, the method includes assessing the subject prior to treatment or first administration and using the results of the assessment to select a subject for treatment. In certain embodiments, the method also includes modifying the administering step (e.g., stopping the administration, increasing or decreasing the periodicity of administration, increasing or decreasing the dose of the α6*nAChR activator) based on the results of the assessment. For example, in embodiments where decreasing a parameter of immune response described herein is desired (e.g., embodiments where a decrease in Th2 cells is desired), the method includes stopping the administration if a marker of Th2 cells is not decreased at least 5%, 10%, 15%, 20%, 30%, 40%, 50% or more; or the method includes increasing the periodicity of administration if the marker of Th2 cells is not decreased at least 5%, 10%, 15%, 20% or more; or the method includes increasing the dose of the α6*nAChR activator if the marker of Th2 cells is not decreased at least 5%, 10%, 15%, 20% or more.

In certain embodiments, immune effects (e.g., immune cell activities) are modulated in a subject (e.g., a subject having an inflammatory or autoimmune condition) or in a cultured cell by at least 1%, 2%, 5%, 10%, 15%, 20%, 25%, 30%, 35%, 40%, 50%, 60%, 70%, 80%, compared to before an administration, e.g., of a dosing regimen, of an α6*nAChR activator such as those described herein. In certain embodiments, the immune effects are modulated in the subject or a cultured cell between 5-20%, between 5-50%, between 10-50%, between 20-80%, between 20-70%, between 50-100%, between 100-500%. The immune effects described herein may be assessed by standard methods:

The α6*nAChR activators described herein are administered in an amount (e.g., an effective amount) and for a time sufficient to effect one of the outcomes described above. The α6*nAChR activator may be administered once or more than once. The α6*nAChR activator may be administered once daily, twice daily, three times daily, once every two days, once weekly, twice weekly, three times weekly, once biweekly, once monthly, once bimonthly, twice a year, or once yearly. Treatment may be discrete (e.g., an injection) or continuous (e.g., treatment via an implant or infusion pump). Subjects may be evaluated for treatment efficacy 1 week, 2 weeks, 1 month, 2 months, 3 months, 4 months, 5 months, 6 months or more following administration of an α6*nAChR activator depending on the α6*nAChR activator and route of administration used for treatment. Depending on the outcome of the evaluation, treatment may be continued or ceased, treatment frequency or dosage may change, or the patient may be treated with a different α6*nAChR activator. Subjects may be treated for a discrete period of time (e.g., 1, 2, 3, 4, 5, 6, 7, 8, 9, 10, 11, or 12 months) or until the disease or condition is alleviated, or treatment may be chronic depending on the severity and nature of the disease or condition being treated.

### Kits

The invention also features a kit including (a) a pharmaceutical composition including an α6*nAChR activator described herein, and (b) instructions for administering the pharmaceutical composition to treat an autoimmune or inflammatory disease or condition.

### EXAMPLES

The following examples are provided to further illustrate some embodiments of the present invention, but are not intended to limit the scope of the invention; it will be understood by their exemplary nature that other procedures, methodologies, or techniques known to those skilled in the art may alternatively be used.

### Example 1—Identification of CHRNA6 on Immune Cells

Natural Tregs were magnetically isolated from human PBMC using a human CD4+CD127low CD25+ regulatory T cell isolation kit (StemCell Technologies). Naïve CD4+ T cells were isolated from human PBMCs using negative magnetic bead selection (Stemcell Technologies). To generate inducible Tregs, naïve CD4+ cells were resuspended in 1 ml of T cell expansion and differentiation media (Stemcell Technologies). Cells were activated with human CD3/CD28 T cell activator (StemCell). Cells were lysed and RNA was extracted (Qiagen).

RNA was sequenced at the Broad Technology Labs (BTL) at the Broad Institute using their Smart-Seq2 protocol, a protocol for full-length transcript sequencing from single cells. Smart-Seq2 libraries were sequenced on a high output sequence machine (Illumina) using a high out-put flow cell and reagent kit to generate 2×25 bp reads (plus dual index reads). Further details are available through the BTL, but in brief, reads were demultiplexed and aligned utilizing an ultrafast RNAseq alignment algorithm (Dobin et al., Bioinformatics. 29:15, 2013) with the following parameters: --twopassMode Basic, --alignIntronMax 1000000, --alignMatesGapMax 1000000, --sjdbScore 2, --quantMode TranscriptomeSAM, and --sjdbOverhang 24.

Quantification of individual read counts was performed using the DESeq2 algorithm (Love et al., Genome Biology 15:550, 2014), a method for differential analysis of count data, using shrinkage estimation for dispersions and fold changes to improve stability and interpretability of estimates. This enabled a more quantitative analysis focused on the strength rather than the mere presence of differential expression. The output of the DESeq2 algorithm was an expression level, in arbitrary units, normalized to an internal factor derived from the sequencing depth of the sample.

Gene expression for CHRNA6 was found to be high in inducible Tregs compared to natural Tregs or PBMCs, as shown in Table 13 below.

TABLE 13
CHRNA6 EXPRESSION IN TREGS

| | | Expression Level |
|---|---|---|
| Cell Type | Gene Name | (DESeq2 normalized) |
| Human PBMCs | CHRNA6 (Entrez: 8973) | 0.185 |
| Human Natural Tregs | CHRNA6 (Entrez: 8973) | 0 |
| Human Inducible | CHRNA6 (Entrez: 8973) | 19.2 |
| Tregs | | |

### Example 2—CHRNA6 Expression in Tregs Correlates with Survival of Cancer Patients

A data set in which T cells (Th1, Th17, Tregs) were isolated from tumors of patients with treatment-naive colorectal cancer (CRC) or non-small-cell lung cancer (NSCLC) was analyzed. The full transcriptional profile of the T cells was analyzed and compared to the transcriptional profile of similar Th1, Th17, and Treg cells isolated from normal tissue or peripheral blood.

The impact of CHRNA6 expression in tumor infiltrating Tregs on survival of cancer patients was analyzed using a clinical history dataset of 177 colorectal cancer patients (GSE17536) and 275 NSCLC patients (GSE41721). Expression of CHRNA6 was normalized to CD3G to account for differential immune infiltration across patients. For each study, an upper (median+STD/10) and lower (median−STD/10) threshold value of CHRNA6 expression was set. Patients in each study were stratified into a “High” CHRNA6 expression group (gene expression at least as high as the upper threshold) or a “Low” CHRNA6 expression group (gene expression less than or equal to the lower threshold). A survival curve was generated for differential expression of CHRNA6 by calculating the number of days from initial pathological diagnosis to death, or if not recorded, then the number of days from initial pathological diagnosis to the last time the patient was reported to be alive.

Patients with higher CHRNA6 expression in Tregs resulted in significantly worse survival in both NSCLC and colorectal cancer, as shown below in Table 14, suggesting that CHRNA6 expression in Tregs promotes their immune regulatory function.

TABLE 14
5 YEAR SURVIVAL IN CANCER PATIENTS WITH
HIGH OR LOW TREG CHRNA6 EXPRESSION

| | 5 Year survival - | 5 Year survival - | |
|---|---|---|---|
| Cancer Type | High CHRNA6 | Low CHRNA6 | P-value |
| NSCLC | 52.8% | 69.2% | P = 0.0034 |
| Colorectal Cancer | 61.3% | 83.1% | P = 0.0019 |

### Example 3—Administration of an α6*nAChR Activator to Treat Local Intestinal Inflammation

According to the methods disclosed herein, a physician of skill in the art can treat a patient, such as a human patient with an inflammatory condition (e.g., intestinal inflammation, such as IBD, ulcerative colitis (UC), or Hirschsprung's disease-associated enterocolitis (HAEC)), so as to reduce the inflammation that contributes to the condition. Before treating the patient, a physician can perform an endoscopy or colonoscopy to diagnose a patient with intestinal inflammation, or identify a patient as having intestinal inflammation based on results from an endoscopy or colonoscopy. To treat the patient, a physician of skill in the art can administer to the human patient an α6*nAChR activator that increases Treg activation (e.g., a small molecule agonist of nAChRs containing a nAChRα6 subunit, e.g., a small molecule listed in Table 1). The small molecule agonist can be administered parenterally (e.g., by subcutaneous injection or intravenous infusion) to treat intestinal inflammation. The small molecule agonist of nAChRs containing a nAChRα6 subunit is administered in a therapeutically effective amount, such as from 10 μg/kg to 500 mg/kg (e.g., 10 μg/kg, 100 μg/kg, 500 μg/kg, 1 mg/kg, 10 mg/kg, 50 mg/kg, 100 mg/kg, 250 mg/kg, or 500 mg/kg). In some embodiments, the small molecule agonist of nAChRs containing a nAChRα6 subunit is administered bimonthly, once a month, once every two weeks, or at least once a week or more (e.g., 1, 2, 3, 4, 5, 6, or 7 times a week or more).

The small molecule agonist of nAChRs containing a nAChRα6 subunit increases Treg production of one or more anti-inflammatory cytokines (e.g., IL-10 or TGFβ). The small molecule agonist of nAChRs containing a nAChRα6 subunit is administered to the patient in an amount sufficient to increase anti-inflammatory cytokine levels by 10% or more (e.g., 10%, 20%, 30%, 40%, 50%, 60%, 70%, 80%, 90%, 95% or more), or improve symptoms of intestinal inflammation (e.g., abdominal pain, diarrhea, fever, and fatigue). Cytokine production can be assessed by collecting a blood sample from the patient and evaluating one or more anti-inflammatory cytokines (e.g., IL-10 or TGFβ). The blood sample can be collected one day or more after administration of the small molecule agonist of nAChRs containing a nAChRα6 subunit (e.g., 1, 2, 3, 4, 5, 6, 7, 10, 14, 21, or 30 or more days after administration). The blood sample can be compared to a blood sample collected from the patient prior to administration of the small molecule agonist of nAChRs containing a nAChRα6 subunit (e.g., a blood sample collected earlier the same day, 1 day, 1 week, 2 weeks, one month or more before administration of the small molecule agonist of nAChRs containing a nAChRα6 subunit). A restoration in intestinal health as evaluated using a colonoscopy, endoscopy or tissue biopsy, reduction in the symptoms of intestinal inflammation (e.g., abdominal pain, diarrhea, fever, and fatigue), a reduction in the markers of intestinal inflammation in a blood sample (e.g., CRP, ESR, calprotectin, or lactoferrin, as compared to levels in a blood sample before treatment), reduced pro-inflammatory cytokine levels, or increased IL-10, TGFβ, Arg1, IDO, PF4, CCL24, or IL4R alpha indicate that the α6*nAChR-specific activating antibody reduces inflammation, increases Treg activation, or treats intestinal inflammation.

### Example 4—Modulation of nAChRs Containing a nAChRα6 Subunit Using Compounds on Inducible Human Tregs

Naïve CD4+ T cells were isolated from human PBMCs using negative magnetic bead selection (Stemcell Technologies). To generate inducible Tregs (iTregs), naïve CD4+ cells were resuspended in 1 ml of T-cell expansion and differentiation media (Stemcell Technologies), 1:50 dilution of Treg differentiation supplement (Stemcell Technologies), 30 ng/mL recombinant human IL-2 (Peprotech), and 100 ng/mL rapamycin (Sigma-Aldrich). Cells were activated with Dynabeads Human T-Activator CD3/CD28 (Invitrogen). Cells were maintained in culture for 7 days to allow for complete differentiation, which was later confirmed by flow cytometry by detecting markers for CD3, CD4, CD25, and FoxP3 on most cells in the population.

To perform the suppressive co-culture assay, iTregs were cultured with CD8+ T-cells isolated from the same human PBMCs using negative magnetic bead selection (Stemcell Technologies). The CD8+ T-cells were isolated 3 days prior to the co-culture and maintained in culture with T-cell expansion and differentiation media (Stemcell Technologies), 30 ng/mL recombinant human IL-2 (Peprotech), and DynaBeads Human T-Activator CD3/CD28 (Invitrogen).

On the day of co-culture, iTregs were combined with CD8+ T-cells. This co-culture was maintained in T-cell expansion and differentiation media (Stemcell Technologies), 30 ng/mL recombinant human IL-2 (Peprotech), and DynaBeads Human T-Activator CD3/CD28 (Invitrogen). Three days after co-culture, cells were processed by flow cytometry to discriminate between the two different populations and intracellular staining of the cytokine IFNγ was used to determine activation of CD8+ T-cells. To determine the effect of compounds on iTreg-mediated immunosuppression of CD8+ T-cell activation, compounds were also added at the beginning of co-culture.

In co-culture, it was found that the addition of α-conotoxin PIA (Tocris) at a final concentration of 3 nM led to a trend of increasing IFNγ+CD8+ T-cells, suggesting that blockade of nAChRs containing a nAChRα6 subunit by this compound impaired the ability of iTregs to suppress CD8+ activity. Conversely, it was found that the addition of nicotine (Tocris) at a final concentration of 2 nM led to a trend of decreasing IFNγ+CD8+ T-cells, suggesting that stimulation of nAChRs containing a nAChRα6 subunit by this compound enhanced the ability of iTregs to suppress CD8+ activity.

Fold change of % IFNγ+CD8+ T-cells in co-culture with compound added relative to % IFNγ+CD8+ T-cells in co-culture without compound are presented per donor for each ratio of CD4:CD8 co-culture condition and shown in Tables 15 and 16 below.

TABLE 15
EFFECT OF ANTAGONIST OF NACHRS CONTAINING
A NACHRα6 SUBUNIT ON IFNγ+
CD8+ T CELLS

| | Fold Change % |
|---|---|
| Donor (CD4+ | IFNγ+ CD8+ |
| T-Cell:CD8+ | Relative to No |
| T-Cell Ratio) | Compound Condition |
| BX26425 (2:1) | 1.48 ± 0.25 |
| BX26425 (1:1) | 2.07 ± 0.18 |
| BX26425 (1:2) | 5.65 ± 1.57 |
| BX26825 (1:1) | 3.05 ± 0.66 |
| BX26825 (1:2) | 2.02 ± 0.16 |
| BX24250 (2:1) | 1.16 ± 0.0058 |
| BX24250 (1:2) | 0.95 ± 0.01 |
| BX23981 (2:1) | 0.93 ± 0.13 |
| BX23981 (1:2) | 1.22 ± 0.042 |

TABLE 16
EFFECT OF AGONIST OF NACHRS CONTAINING A NACHRα6
SUBUNIT ON IFNγ+ CD8+ T CELLS

| | Fold Change % |
|---|---|
| Donor (CD4+ | IFNγ+ CD8+ |
| T-Cell:CD8+ | Relative to No |
| T-Cell Ratio) | Compound Condition |
| BX26425 (2:1) | 0.18 ± 0.11 |
| BX26425 (1:1) | 0.31 ± 0.089 |
| BX26425 (1:2) | 0.42 ± 0.044 |
| BX26825 (1:1) | 0.70 ± 0.10 |
| BX26825 (1:2) | 0.91 ± 0.14 |
| BX24250 (2:1) | 1.07 ± 0.046 |
| BX24250 (1:2) | 0.84 ± 0.10 |
| BX23981 (2:1) | 0.88 ± 0.035 |
| BX23981 (1:2) | 1.20 ± 0.038 |
| BX27275 (2:1) | 0.9 ± 0.12 |
| BX27275 (1:1) | 1.16 ± 0.12 |
| BX27275 (2:1) | 1.38 ± 0.41 |

### Example 5—Knockout of CHRNA6 in Inducible Human Tregs Affects their Immunosuppressive Potency

Naïve CD4+ T cells were isolated from human PBMCs using negative magnetic bead selection (Stemcell Technologies). To generate inducible Tregs (iTregs), naïve CD4+ cells were resuspended in 1 ml of T-cell expansion and differentiation media (Stemcell Technologies), 1:50 dilution of Treg differentiation supplement (Stemcell Technologies), 30 ng/mL recombinant human IL-2 (Peprotech), and 100 ng/mL rapamycin (Sigma-Aldrich). Cells were activated with Dynabeads Human T-Activator CD3/CD28 (Invitrogen). Cells were maintained in culture for 7 days to allow for complete differentiation, which was later confirmed by flow cytometry by detecting markers for CD3, CD4, CD25, and FoxP3 on most cells in the population.

To perform the suppressive co-culture assay, iTregs were cultured with CD8+ T-cells isolated from the same human PBMCs using negative magnetic bead selection (Stemcell Technologies). The CD8+ T-cells were isolated 3 days prior to the co-culture and maintained in culture with T-cell expansion and differentiation media (Stemcell Technologies), 30 ng/mL recombinant human IL-2 (Peprotech), and DynaBeads Human T-Activator CD3/CD28 (Invitrogen).

On the day of co-culture, iTregs were combined with CD8+ T-cells. This co-culture was maintained in T-cell expansion and differentiation media (Stemcell Technologies), 30 ng/mL recombinant human IL-2 (Peprotech), and DynaBeads Human T-Activator CD3/CD28 (Invitrogen). Three days after co-culture, cells were processed by flow cytometry to discriminate between the two different populations and intracellular staining of the cytokine IFNγ was used to determine activation of CD8+ T-cells.

To determine the effect of knockout of CHRNA6 on iTreg-mediated immunosuppression of CD8+ T-cell activation, CHRNA6 was knocked out using nucleofection. This process involved mixing the isolated Naïve CD4+ T-cells on the day of isolation with P4 Buffer (Lonza), Recombinant Cas9 (Life Tehcnologies), and 3 unique sgRNA for CHRNA6 (Synthego) and performing the nucleofection procedure with program CM137 using the 4D-Nucleofector (Lonza). The cells were then cultured as described above. The iTregs with CHRNA6 knocked out were then co-cultured as previously described to determine the effect of knocking out CHRNA6 on CD8+ immunosuppression by iTregs.

For knockout of CHRNA6, the 3 sgRNA sequences were combined. The sgRNA had the sequences: G U U U G G C C U C A C A G G C U G U G (SEQ ID NO: 1), C U G U G U G G G C U G U G C A A C U G (SEQ ID NO: 2), and U G G G C U G U G C A A C U G A G G A G (SEQ ID NO: 3).

It was found that when CHRNA6 was knocked out in iTregs, there was a trend of increasing IFNγ+ in CD8+ T-cells, suggesting immunosuppression by iTregs was reduced in the absence of CHRNA6.

Percent IFNγ+CD8+ T-cells in co-culture with Tregs nucleofected with either negative control KO or CHRNA6 KO are presented in Table 17 below.

TABLE 17
EFFECT OF CHRNA6 KNOCKOUT ON IFNγ
IN CD8+ T CELLS

| Donor | Co-culture Condition | % IFNγ+ CD8+ |
|---|---|---|
| BX28521 | Negative KO in iTregs | 1.62 ± 0.28 |
| BX28521 | CHRNA6 KO in iTregs | 2.05 ± 0.46 |
| BX28480 | Negative KO in iTregs | 1.47 ± 0.36 |
| BX28480 | CHRNA6 KO in iTregs | 2.12 ± 0.64 |

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims. Other embodiments are within the claims.

1. A method of modulating an immune response in a subject in need thereof, the method comprising administering an effective amount of an activator of nicotinic acetylcholine receptors (nAChRs) containing a nicotinic alpha 6 subunit (α6*nAChR activator).

2. A method of modulating an immune response in a subject in need thereof, the method comprising contacting an immune cell, spleen, lymph node, secondary lymphoid organ, tertiary lymphoid organ, barrier tissue, skin, gut, or airway with an effective amount of an α6*nAChR activator.

3. A method of modulating an immune cell activity in a subject in need thereof, the method comprising contacting an immune cell with an effective amount of an α6*nAChR activator.

4. A method of treating a subject with an inflammatory or autoimmune disease or condition, the method comprising administering to the subject an effective amount of an α6*nAChR activator.

5. A method of treating a subject with an inflammatory or autoimmune disease or condition, the method comprising contacting an immune cell, spleen, lymph node, secondary lymphoid organ, tertiary lymphoid organ, barrier tissue, skin, gut, or airway with an effective amount of an α6*nAChR activator.

6. A method of treating a subject identified as having an inflammatory or autoimmune disease or condition, the method comprising administering to the subject an effective amount of an α6*nAChR activator.

7. A method of treating a subject identified as having an inflammatory or autoimmune disease or condition, the method comprising contacting an immune cell, spleen, lymph node, secondary lymphoid organ, tertiary lymphoid organ, barrier tissue, skin, gut, or airway with an effective amount of an α6*nAChR activator.

8. The method of any one of claims 4-7, wherein the inflammatory or autoimmune disease or condition is an α6*nAChR-associated inflammatory or autoimmune disease or condition.

9. A method of treating a subject with an inflammatory or autoimmune disease or condition, the method comprising: a) identifying a subject with an α6*nAChR-associated inflammatory or autoimmune disease or condition; and b) administering to the subject an effective amount of an α6*nAChR activator.

10. A method of treating a subject with an inflammatory or autoimmune disease or condition, the method comprising: a) identifying a subject with an α6*nAChR-associated inflammatory or autoimmune disease or condition; and b) contacting an immune cell, spleen, lymph node, secondary lymphoid organ, tertiary lymphoid organ, barrier tissue, skin, gut, or airway with an effective amount of an α6*nAChR activator.

11. A method of treating a subject with an α6*nAChR-associated inflammatory or autoimmune disease or condition, the method comprising administering to the subject an effective amount of an α6*nAChR activator.

12. A method of treating a subject with an α6*nAChR-associated inflammatory or autoimmune disease or condition, the method comprising contacting an immune cell, spleen, lymph node, secondary lymphoid organ, tertiary lymphoid organ, barrier tissue, skin, gut, or airway with an effective amount of an α6*nAChR activator.

13. The method of any one of claim 2, 5, 7, 10, or 12, wherein the method comprises contacting an immune cell with an effective amount of an α6*nAChR activator.

14. The method of any one of claim 2, 5, 7, 10, or 12, wherein the method comprises contacting a spleen with an effective amount of an α6*nAChR activator.

15. The method of any one of claim 2, 5, 7, 10, or 12, wherein the method comprises contacting a lymph node with an effective amount of an α6*nAChR activator.

16. The method of any one of claim 2, 5, 7, 10, or 12, wherein the method comprises contacting a secondary lymphoid organ with an effective amount of an α6*nAChR activator.

17. The method of any one of claim 2, 5, 7, 10, or 12, wherein the method comprises contacting a tertiary lymphoid organ with an effective amount of an α6*nAChR activator.

18. The method of any one of claim 2, 5, 7, 10, or 12, wherein the method comprises contacting a barrier tissue with an effective amount of an α6*nAChR activator.

19. The method of any one of claim 2, 5, 7, 10, or 12, wherein the method comprises contacting the skin with an effective amount of an α6*nAChR activator.

20. The method of any one of claim 2, 5, 7, 10, or 12, wherein the method comprises contacting the gut with an effective amount of an α6*nAChR activator.

21. The method of any one of claim 2, 5, 7, 10, or 12, wherein the method comprises contacting an airway with an effective amount of an α6*nAChR activator.

22. The method of any one of claims 4-21, wherein the α6*nAChR-associated inflammatory or autoimmune disease or condition is associated with expression of α6*nAChR in an immune cell.

23. The method any one of claims 1-22, wherein the method comprises contacting an immune cell with an effective amount of an α6*nAChR activator that increases α6*nAChR expression or activity.

24. The method of any one of claims 1-23, wherein the method comprises modulating an immune cell activity.

25. The method of claim 3 or 24, wherein the immune cell activity is activation, proliferation, polarization, cytokine production, recruitment, or migration.

26. The method of claim 25, wherein the activation, proliferation, polarization, cytokine production, recruitment, or migration is increased.

27. The method of claim 26, wherein cytokine production is increased.

28. The method of claim 27, wherein the cytokine is an anti-inflammatory cytokine.

29. The method of claim 26, wherein activation is increased.

30. The method of claim 26, wherein migration is directed toward a site of inflammation.

31. A method of increasing regulatory T cell (Treg) cytokine production in a subject in need thereof, the method comprising contacting a Treg with an effective amount of an α6*nAChR activator.

32. A method of increasing Treg cytokine production in a subject in need thereof, the method comprising administering to the subject an effective amount of an α6*nAChR activator.

33. The method of claim 31 or 32, wherein Treg production of anti-inflammatory cytokines is increased.

34. The method of claim 33, wherein the anti-inflammatory cytokine is interleukin-10 (IL-10) or transforming growth factor beta (TGFβ).

35. The method of claim 33 or 34, wherein T cell cytokine production of pro-inflammatory cytokines is decreased.

36. A method of decreasing pro-inflammatory cytokine levels in a subject in need thereof, the method comprising administering to the subject an effective amount of an α6*nAChR activator.

37. A method of decreasing T cell pro-inflammatory cytokine production in a subject in need thereof, the method comprising administering to the subject an effective amount of an α6*nAChR activator.

38. The method of any one of claims 35-37, wherein the pro-inflammatory cytokine is interferon gamma (IFNγ).

39. A method of decreasing T cell activation in a subject in need thereof, the method comprising administering to the subject an effective amount of an α6*nAChR activator.

40. The method of any one of claims 1-39, wherein the method further comprises contacting an immune cell isolated from the subject with an α6*nAChR activator and evaluating the response of the immune cell prior to administration of the α6*nAChR activator.

41. A method of treating a subject with an inflammatory or autoimmune disease or condition, the method comprising a) contacting an immune cell isolated from the subject with an α6*nAChR activator and evaluating a response of the immune cell; and b) administering to the subject an effective amount of an α6*nAChR activator.

42. A method of predicting the response of an inflammatory or autoimmune disease or condition in a subject to treatment with an α6*nAChR activator, the method comprising contacting an immune cell isolated from the subject with an α6*nAChR activator and evaluating the response of the immune cell.

43. The method of any one of claims claim 40-42, wherein the evaluating comprises assessing immune cell migration, immune cell proliferation, immune cell recruitment, immune cell differentiation, immune cell activation, immune cell polarization, immune cell cytokine production, immune cell degranulation, immune cell maturation, immune cell antibody-dependent cell-mediated cytotoxicity (ADCC), immune cell antibody-dependent cell-mediated phagocytosis (ADCP), immune cell antigen presentation, and/or immune cell nAChRα6 expression.

44. The method of claim 43, wherein the immune cell is a Treg.

45. The method of claim 44, wherein the evaluating comprises evaluating Treg activation.

46. The method of claim 44, wherein the evaluating comprises evaluating Treg cytokine production.

47. The method of claim 46, wherein the cytokine is an anti-inflammatory cytokine.

48. The method of claim 47, wherein the anti-inflammatory cytokine is IL-10 or TGFβ.

49. A method of predicting the response of an inflammatory or autoimmune disease or condition in a subject to treatment with an α6*nAChR activator, the method comprising: a) isolating an immune cell from the subject; b) measuring the expression of nAChRα6 in the immune cell; and c) comparing nAChRα6 expression in the immune cell to a reference, wherein decreased expression of nAChRα6 in the immune cell as compared to the reference indicates that the subject will respond to treatment with an α6*nAChR activator.

50. A method of characterizing an inflammatory or autoimmune disease or condition in a subject, the method comprising: a) isolating an immune cell from the subject; b) measuring the expression of nAChRα6 in the immune cell; and c) comparing nAChRα6 expression in the immune cell to a reference, wherein decreased expression of nAChRα6 in the immune cell as compared to the reference indicates that the subject has an α6*nAChR-associated inflammatory or autoimmune disease or condition.

51. A method of identifying a subject as having an α6*nAChR-associated inflammatory or autoimmune disease or condition, the method comprising: a) isolating an immune cell from the subject; b) measuring the expression of nAChRα6 in the immune cell; and c) comparing nAChRα6 expression in the immune cell to a reference, wherein decreased expression of nAChRα6 in the immune cell as compared to the reference indicates that the subject has a nAChRα6-associated inflammatory or autoimmune disease or condition.

52. The method of any one of claims 49-51, wherein the method further comprises providing an α6*nAChR activator suitable for administration to the subject.

53. The method of any one of claims 49-51, wherein the method further comprises administering to the subject an effective amount of an α6*nAChR activator.

54. The method of any one of claims 1-53, wherein the α6*nAChR activator increases α6*nAChR expression or activation.

55. The method of claim 54, wherein the α6*nAChR activator induces or increases channel opening.

56. The method of claim 54 or 55, wherein the α6*nAChR activator stabilizes the channel in an open conformation.

57. The method of any one of claims 4-56, wherein the inflammatory or autoimmune disease or condition is systemic lupus erythematosus (SLE), rheumatoid arthritis, multiple sclerosis (MS), irritable bowel disorder (IBD), Crohn's disease, ulcerative colitis, dermatitis, psoriasis, or asthma.

58. The method of claim 57, wherein the inflammatory or autoimmune disease or condition is SLE.

59. The method of claim 57, wherein the inflammatory or autoimmune disease or condition is rheumatoid arthritis.

60. The method of claim 57, wherein the inflammatory or autoimmune disease or condition is rheumatoid MS.

61. The method of claim 57, wherein the inflammatory or autoimmune disease or condition is IBD.

62. The method of claim 57, wherein the inflammatory or autoimmune disease or condition is rheumatoid Crohn's disease.

63. The method of claim 57, wherein the inflammatory or autoimmune disease or condition is ulcerative colitis.

64. The method of claim 57, wherein the inflammatory or autoimmune disease or condition is psoriasis.

65. The method of any one of claims 4-56, wherein the inflammatory or autoimmune disease or condition is an IFNγ-associated inflammatory or autoimmune disease or condition.

66. The method of any one of claims 4-56, wherein the inflammatory or autoimmune disease or condition is an inflammatory or autoimmune disease or condition associated with activated T cells.

67. The method of any one of claims 1-66, wherein the α6*nAChR activator is administered locally.

68. The method of claim 67, wherein the α6*nAChR activator is administered to or near a lymph node, the spleen, a secondary lymphoid organ, a tertiary lymphoid organ, a barrier tissue, skin, the gut, or an airway.

69. The method of any one of claims 1-68, wherein the method further comprises administering a second therapeutic agent.

70. The method of claim 69, wherein the second therapeutic agent is a disease-modifying anti-rheumatic drug (DMARD), a biologic response modifier (a type of DMARD), a corticosteroid, a nonsteroidal anti-inflammatory medication (NSAID), prednisone, prednisolone, methylprednisolone, methotrexate, hydroxycholorquine, sulfasalazine, leflunomide, cyclophosphamide, azathioprine, tofacitinib, adalimumab, abatacept, anakinra, kineret, certolizumab, etanercept, golimumab, infliximab, rituximab tocilizumab, an antiviral compound, a nucleoside-analog reverse transcriptase inhibitor (NRTI), a non-nucleoside reverse transcriptase inhibitor (NNRTI), an antibacterial compound, an antifungal compound, an antiparasitic compound, 6-mercaptopurine, 6-thioguanine, alemtuzumab, aminosalicylates, antibiotics, anti-histamines, anti-TNFα, azathioprine, belimumab, beta interferon, calcineurin inhibitors, certolizumab, corticosteroids, cromolyn, cyclosporin A, cyclosporine, dimethyl fumarate, fingolimod, fumaric acid esters, glatiramer acetate, hydroxyurea, IFNγ, IL-11, leflunomide, leukotriene receptor antagonist, long-acting beta2 agonist, mitoxantrone, mycophenolate mofetil, natalizumab, ocrelizumab, pimecrolimus, probiotics, retinoids, salicylic acid, short-acting beta2 agonist, sulfasalazine, tacrolimus, teriflunomide, theophylline, ustekinumab, vedolizumab, an second α6*nAChR activator, a neurotransmission modulator, or a neuronal growth factor modulator.

71. The method of any one of claims 1-70, wherein the α6*nAChR activator is selected from the group consisting of an antibody and a small molecule.

72. The method of claim 71, wherein the α6*nAChR activator is an antibody.

73. The method of claim 72, wherein the antibody is an α6*nAChR activating antibody or an antigen binding fragment thereof.

74. The method of claim 71, wherein the α6*nAChR activator is a small molecule.

75. The method of claim 74, wherein the small molecule is a small molecule activator α6*nAChR activator.

76. The method of claim 75, wherein the small molecule α6*nAChR activator is a small molecule activator listed in Table 1.

77. The method of any one of claims 1-76, wherein the α6*nAChR activator does not cross the blood brain barrier.

78. The method of claim 77, wherein the α6*nAChR activator has been modified to prevent blood brain barrier crossing by conjugation to a targeting moiety, formulation in a particulate delivery system, addition of a molecular adduct, or through modulation of its size, polarity, flexibility, or lipophilicity.

79. The method of any one of claims 1-78, wherein the α6*nAChR activator does not have a direct effect on the central nervous system or gut.

80. The method of any one of claims 1-79, wherein the α6*nAChR activator increases organ function, increases immune cell migration, increases immune cell proliferation, increases immune cell recruitment, increases immune cell activation, increases immune cell polarization, increases immune cell cytokine production, increases immune cell α6*nAChR expression, decreases inflammation, decreases auto-antibody levels, and/or decreases the rate or number of relapses or flare-ups.

81. The method of claim 80, wherein the α6*nAChR activator increases immune cell activation.

82. The method of claim 80, wherein the α6*nAChR activator increases immune cell cytokine production.

83. The method of any one of claims 1-82, wherein the method further comprises measuring one or more of the development of high endothelial venules (HEVs) or tertiary lymphoid organs (TLOs), immune cell migration, immune cell proliferation, immune cell recruitment, immune cell differentiation, immune cell activation, immune cell polarization, immune cell cytokine production, immune cell ADCC, immune cell ADCP, symptoms of an autoimmune or inflammatory condition, inflammation, auto-antibody levels, organ function, the rate or number of relapses or flare-ups, and/or immune cell nAChRα6 expression before administration of the α6*nAChR activator.

84. The method of claim 83, wherein the method further comprises measuring immune cell activation before administration of the α6*nAChR activator.

85. The method of claim 83, wherein the method further comprises measuring immune cell cytokine production before administration of the α6*nAChR activator.

86. The method of any one of claims 1-83, wherein the method further comprises measuring one or more of the development of HEVs or TLOs, immune cell migration, immune cell proliferation, immune cell recruitment, immune cell differentiation, immune cell activation, immune cell polarization, immune cell cytokine production, immune cell ADCC, immune cell ADCP, symptoms of an autoimmune or inflammatory condition, inflammation, auto-antibody levels, organ function, the rate or number of relapses or flare-ups, or immune cell nAChRα6 expression after administration of the α6*nAChR activator.

87. The method of claim 80, wherein the method further comprises measuring immune cell activation after administration of the α6*nAChR activator.

88. The method of claim 80, wherein the method further comprises measuring immune cell cytokine production after administration of the α6*nAChR activator.

89. The method of any one of claims 1-88, wherein the α6*nAChR activator is administered in an amount sufficient to increase immune cell migration, increase immune cell proliferation, increase immune cell recruitment, increase immune cell activation, increase immune cell polarization, increase immune cell cytokine production, increase immune cell α6*nAChR expression, treat the autoimmune or inflammatory condition, reduce symptoms of an autoimmune or inflammatory condition, reduce inflammation, reduce auto-antibody levels, increase organ function, and/or decrease the rate or number of relapses or flare-ups.

90. The method of claim 89, wherein the α6*nAChR activator is administered in an amount sufficient to increase immune cell activation.

91. The method of claim 89, wherein the α6*nAChR activator is administered in an amount sufficient to increase immune cell cytokine production.

92. The method of any one of claims 80-91, wherein the cytokine is one or more of IL-10 or TGFβ.

93. The method of any one of claim 2, 3, 5, 7, 10, 12, 13, 22-34, or 40-92, wherein the immune cell is a Treg.

94. A therapy for treating an anti-inflammatory or autoimmune disease or condition comprising an α6*nAChR activator and a second agent selected from the group consisting of a DMARD, a biologic response modifier (a type of DMARD), a corticosteroid, an NSAID, prednisone, prednisolone, methylprednisolone, methotrexate, hydroxycholorquine, sulfasalazine, leflunomide, cyclophosphamide, azathioprine, tofacitinib, adalimumab, abatacept, anakinra, kineret, certolizumab, etanercept, golimumab, infliximab, rituximab tocilizumab, an antiviral compound, an NRTI, an NNRTI, an antibacterial compound, an antifungal compound, an antiparasitic compound, 6-mercaptopurine, 6-thioguanine, alemtuzumab, aminosalicylates, antibiotics, anti-histamines, anti-TNFα, azathioprine, belimumab, beta interferon, calcineurin inhibitors, certolizumab, corticosteroids, cromolyn, cyclosporin A, cyclosporine, dimethyl fumarate, fingolimod, fumaric acid esters, glatiramer acetate, hydroxyurea, IFNγ, IL-11, leflunomide, leukotriene receptor antagonist, long-acting beta2 agonist, mitoxantrone, mycophenolate mofetil, natalizumab, ocrelizumab, pimecrolimus, probiotics, retinoids, salicylic acid, short-acting beta2 agonist, sulfasalazine, tacrolimus, teriflunomide, theophylline, ustekinumab, vedolizumab, a neurotransmission modulator, and a neuronal growth factor modulator.

95. The therapy of claim 94, wherein the α6*nAChR activator is an α6*nAChR activating antibody or an antigen binding fragment thereof.

96. The therapy of claim 94, wherein the α6*nAChR activator is a small molecule α6*nAChR activator.

97. The therapy of claim 96, wherein the small molecule activator is a small molecule activator listed in Table 1.

98. A pharmaceutical composition comprising an α6*nAChR activating antibody or an antigen binding fragment thereof.

99. The pharmaceutical composition of claim 98, wherein the α6*nAChR activating antibody agonizes α6*nAChR.

100. The pharmaceutical composition of claim 98 or 99, wherein the α6*nAChR activating antibody stabilizes the channel in an open conformation.

101. The pharmaceutical composition of any one of claims 98-100, wherein the composition further comprises a second therapeutic agent.

102. The pharmaceutical composition of claim 101, wherein the second therapeutic agent is a DMARD, a biologic response modifier (a type of DMARD), a corticosteroid, an NSAID, prednisone, prednisolone, methylprednisolone, methotrexate, hydroxycholorquine, sulfasalazine, leflunomide, cyclophosphamide, azathioprine, tofacitinib, adalimumab, abatacept, anakinra, kineret, certolizumab, etanercept, golimumab, infliximab, rituximab tocilizumab, an antiviral compound, an NRTI, an NNRTI, an antibacterial compound, an antifungal compound, an antiparasitic compound, 6-mercaptopurine, 6-thioguanine, alemtuzumab, aminosalicylates, antibiotics, anti-histamines, anti-TNFα, azathioprine, belimumab, beta interferon, calcineurin inhibitors, certolizumab, corticosteroids, cromolyn, cyclosporin A, cyclosporine, dimethyl fumarate, fingolimod, fumaric acid esters, glatiramer acetate, hydroxyurea, IFNγ, IL-11, leflunomide, leukotriene receptor antagonist, long-acting beta2 agonist, mitoxantrone, mycophenolate mofetil, natalizumab, ocrelizumab, pimecrolimus, probiotics, retinoids, salicylic acid, short-acting beta2 agonist, sulfasalazine, tacrolimus, teriflunomide, theophylline, ustekinumab, vedolizumab, a second α6*nAChR activator, a neurotransmission modulator, or a neuronal growth factor modulator.

103. The pharmaceutical composition of any one of claims 98-102, wherein the composition further comprises a pharmaceutically acceptable excipient.


SEQUENCE LISTING (3 sequences)

SEQ ID NO: 1 - RNA, 20 nt, Artificial Sequence
  Synthetic Construct
guuuggccucacaggcugug

SEQ ID NO: 2 - RNA, 20 nt, Artificial Sequence
  Synthetic Construct
cugugugggcugugcaacug

SEQ ID NO: 3 - RNA, 20 nt, Artificial Sequence
  Synthetic Construct
ugggcugugcaacugaggag